(19) United States
(12) Patent Application Publication
(10) Pub. No.: US 2025/0352508 A1
(43) Pub. Date: Nov. 20, 2025

(54) PREVENTION AND TREATMENT OF POST-ACUTE INFECTION SYNDROMES

(71) Applicants: InflammX Therapeutics, Inc., San Diego, CA (US); Auckland Uniservices Limited, Auckland (NZ)

(72) Inventors: Brian LEVY, Tampa, FL (US); David POOL, San Diego, CA (US); Anthony Ronald John PHILLIPS, Auckland (NZ); Colin Richard GREEN, Auckland (NZ)

(21) Appl. No.: 19/208,423

(22) Filed: May 14, 2025

Related U.S. Application Data
(60) Provisional application No. 63/647,539, filed on May 14, 2024.

(51) Int. Cl. A61K 31/352; A61P 31/14

(52) U.S. Cl. CPC A61K 31/352; A61P 31/14

(57) ABSTRACT

The inventions relate to compounds and compositions comprising connexin hemichannel modulators, inflammasome modulators, and/or pannexin channel modulators and methods for the treatment of post-acute infection disorders, including long COVID, long flu, and post-treatment Lyme disease syndrome.

### RELATED APPLICATIONS

This application claims the benefit of priority to U.S. Ser. No. 63/647,539, filed on May 14, 2024, which is incorporated herein in its entirety.

### FIELD

The inventions generally to compounds and compositions used for treating and/or preventing post-acute infection syndromes, including long-term sequalae of coronavirus infection and signs and symptoms of long-haul effects resulting from other infections.

### INCORPORATION BY REFERENCE

All publications, patents, related applications, and other written or electronic materials mentioned, identified or referred to herein, including each and every United States patent, United States patent application publication, non-U.S. patent, non-U.S. and PCT published application, article and other document cited or noted herein, and all those listed as References Cited in any patent or patents that issue herefrom, are hereby incorporated by reference in their entirety. The information incorporated is as much a part of this application, and all patents issuing therefrom or claiming priority thereto, as if all of the text and other content was repeated in the application or patent, and will be treated as part of the text and content of this application as filed and any patent issuing therefrom or claiming priority thereto, and any portion of any material incorporated by reference may be included herein by amendment if required or desired. In the event of inconsistent usages between this document and any document incorporated by reference, the usage in the incorporated reference(s) should be considered supplementary to that of this document; for irreconcilable inconsistencies, including definitions appearing in patents or patent applications, the usage in this document controls.

### SEQUENCE LISTING

The instant application contains a Sequence Listing which has been submitted electronically and is hereby incorporated by reference in its entirety. Said file is named E3697-00623_SL.xml created on May 13, 2025, and is about 275,181 bytes in size.

### TECHNICAL INTRODUCTION

The following includes information that may be useful in providing some background to the present inventions. It is not an admission that any of the information, publications or documents specifically or implicitly referenced herein are prior art, or important, to the inventions described and claimed herein. It is not a suggestion that the brief introductory descriptions are comprehensive. Many scientific articles have been written about these specialized areas and technical disciplines, as well as books and book chapters.

Long COVID refers to an array of signs and symptoms that sometimes linger weeks and months, even years, after the person survives a COVID infection. Sometimes the symptoms can go away and come back again. These long-term effects have been documented in the literature. See, e.g., Al-Aly Z, et al. High-dimensional characterization of post-acute sequelae of COVID-19. Nature 594(7862):259-264 (2021); Del Rio C, et al. Long-term Health Consequences of COVID-19. JAMA 324(17):1723-1724 (2020); Huang C, et al. 6-month consequences of COVID-19 in patients discharged from hospital: a cohort study. Lancet 397(10270):220-232 (2021; Taquet M, et al. 6-month neurological and psychiatric outcomes in 236 379 survivors of COVID-19: a retrospective cohort study using electronic health records. Lancet Psychiatry 8(5):416-427 (2021); Wanga V, et al., Long-Term Symptoms Among Adults Tested for SARS-CoV-2—United States, January 2020-April 2021. MMWR Morb Mortal Wkly Rep 70(36):1235-1241 (2021); Lopez-Leon S, et al. More than 50 long-term effects of COVID-19: a systematic review and meta-analysis. Sci R ep 11(1):16144 (2021); Carfi A, Bernabei R, Landi F, for the Gemelli Against COVID-19 Post-Acute Care Study Group. Persistent Symptoms in Patients After Acute COVID-19. JAMA 324(6):603-605 (2020).

Various names have been used to describe this condition, including long COVID, long-haulers, long-term effects of COVID-19, post-COVID syndrome, chronic COVID syndrome, post-COVID conditions, and post-acute sequelae SARS-CoV-2 infection (PASC). The term long COVID is used in this patent.

Symptoms reported by long COVID patients range from fatigue, dyspnea, loss of smell to “brain fog” and neuropathy. According to the CDC, other symptoms include those that get worse after physical or mental effort (also known as “post-exertional malaise’), as well as fever, respiratory and heart symptoms including difficulty breathing or shortness of breath, cough, chest pain, fast-beating or pounding heart (also known as heart palpitations), neurological symptoms including difficulty thinking or concentrating (sometimes referred to as “brain fog”), headache, sleep problems, dizziness upon standing up (lightheadedness), pins-and-needles feelings, change in smell or taste, depression or anxiety, digestive symptoms including diarrhea and stomach pain, and other symptoms including joint or muscle pain, rash and changes in menstrual cycles.

The incidence of long COVID varies widely between studies, with the majority between about 10% and 30%. See, e.g., Fung K W, et al. Prevalence and characteristics of long COVID in elderly patients: An observational cohort study of over 2 million adults in the US. PLoS Med 20(4):e1004194 (2023). According to one estimate, up to 23 million people in the United States may have developed long COVID as of February 2022. U.S. Government Accountability Office. Science & Tech Spotlight: Long COVID. Available from: https://www.gao.gov/products/gao-22-105666?utm_source=onepager&utm_medium=email&utm_campaign=email_staa. Another study estimated that at least 3 to 5 million US adults have activity-limiting long COVID. Tenforde M W, et al. Point Prevalence Estimates of Activity-Limiting Long-term Symptoms Among United States Adults ≥1 Month After Reported Severe Acute Respiratory Syndrome Coronavirus 2 Infection, 1 Nov. 2021. J Infect Dis 227(7):855-863 (2023). More recently, authors of a paper published in Nature noted that there are at least 65 million long COVID cases worldwide, although they acknowledge this is a conservative estimate based on an incidence rate of 10% of the 651 million documented cases of COVID-19 worldwide. Davis. H. E., et at Long COVID: major findings, mechanisms and recommendations. Nat Rev Microbiol 21, 133-146 (2023).

SARS-CoV-2 viruses are not unique in their ability to cause post-acute sequelae. Certain acute infections have long been associated with an unexplained chronic disability in a minority of patients. All manner of infectious agents, including bacteria, viruses, and parasites, has been implicated in the pathogenesis of post-acute infection syndromes, which include “long flu,” “long cold,” and post-treatment Lyme disease syndrome.

Among well-established post-acute infection syndromes is Q fever fatigue syndrome, which follows infection by the intracellular bacterium Coxiella burnetiid. Morroy, G., et al. Fatigue following acute Q-fever: a systematic literature review. PLoS One 11: e0155884 (2016). This syndrome lingers in a minority of Q fever survivors for prolonged periods and is associated with substantial morbidity. Another post-acute infection syndrome with significant worldwide impact is post-dengue fatigue syndrome, which can follow infection by the mosquito-borne dengue virus. Hung, T. M., et al. The uncertainty surrounding the burden of post-acute consequences of dengue infection. Trends Parasitol. 35: 673-676 (2019). Another post-acute infection syndrome is post-Ebola syndrome. Carod-Artal, F. J. Post-Ebola virus disease syndrome: what do we know? Expert Rev. Anti. Infect. Ther. 13: 1185-1187 (2015). In the past 10 years, substantial evidence has been presented for a post-acute infection syndrome with fatiguing and rheumatic symptoms in a subset of individuals infected with chikungunya virus, a mosquito-borne virus that causes fever and joint pain in the acute phase. Rodriguez-Morales, A. J., et al. Prevalence of post-chikungunya infection chronic inflammatory arthritis: a systematic review and meta-analysis. Arthritis Care Res. 68: 1849-1858 (2016); Paixio, E. S., et al. Chikungunya chronic disease: a systematic review and meta-analysis. Trans. R. Soc. Trop. Med. Hyg. 112: 301-316 (2018). Evidence also indicates the possibility of similar post-acute symptoms following infections with other arthritogenic alphaviruses, such as Ross River virus and West Nile virus. Hickie, I., et al. Post-infective and chronic fatigue syndromes precipitated by viral and non-viral pathogens: Prospective cohort study. Br. Med. J. 333: 575-578 (2006).

Prolonged, debilitating, chronic symptoms have long been reported in a subset of patients after common infections including for example after mononucleosis, a condition generally caused by Epstein-Barr virus (EBV) and after an outbreak of Giardia lamblia, an intestinal parasite that usually causes acute intestinal illness. Several studies identified the association of this outbreak of giardiasis with chronic fatigue (Naess, H., et al. Chronic Fatigue Syndrome after Giardia enteritis: clinical characteristics, disability and long-term sickness absence. BMC Gastroenterol. 12: 13 (2012)), irritable bowel syndrome (IBS)(Litleskare, S., et al. Prevalence of irritable bowel syndrome and chronic fatigue 10 years after Giardia infection. Clin. Gastroenterol. Hepatol. 16: 1064-1072.e4 (2018)); and fibromyalgia (Hunskar, G. S., et al. Prevalence of fibromyalgia 10 years after infection with Giardia lamblia: a controlled prospective cohort study. Scand. J. Pain 22: 348-355 (2021)) persisting for many years. Another post-acute infection syndrome is post-treatment Lyme disease syndrome, which can present as persistent arthralgia, fatigue, and neurocognitive impairments in a minority of patients with Lyme disease after the recommended antibiotic treatment. Rebman, A. W. & Aucott, J. N. Post-treatment Lyme disease as a model for persistent symptoms in Lyme disease. Front. Med. 7: 57 (2020). Other studies have evaluated ME/CFS (myalgic encephalomyelitis/chronic fatigue syndrome) for chronic post-infection sequelae.

Evidence also points to “long flu”, with a large study showing symptoms persist at least 4 weeks or more after some people are hospitalized for the flu. See, e.g., Fung K W, et al. Prevalence and characteristics of long COVID in elderly patients: An observational cohort study of over 2 million adults in the US. PLoS Med 20(4):e1004194 (2023). Common symptoms among people with “long colds” were coughing, stomach pain, and diarrhea. See Vivaldi, G., et al., Long-term symptom profiles after COVID-19 vs other acute respiratory infections: an analysis of data from the COVIDENCE UK study, eClinicalMedicine 65:102251 (2023). The CDC estimated that 1 in 10 people who get COVID go on to experience long COVID, which was defined in that estimate as symptoms lasting at least 3 months. For this new study comparing long COVID to long colds, the definition was symptoms lasting at least 4 weeks. People with long COVID were more likely than people with long colds to experience problems with taste and smell, hair loss, unusual sweating, higher heart rate, and memory problems.

The terms post-infectious fatigue syndrome and post-viral fatigue syndrome are sometimes used to describe debilitating fatigue following an infection, often accompanied by other signs and symptoms. Post-infectious and post-viral fatigue syndromes were originally postulated as subsets of ‘chronic fatigue syndrome,’ in which the triggering infectious agent is objectively documented67. However, there appears to be no clear consensus at present about the distinctions across these concepts. It is thus unclear whether these terms should be considered synonymous to the ME/CFS label, any of its subsets, or include a wider range of post-infectious fatigue conditions.

Gap junctions are specialized intercellular connections found between most animal cell-types. They are expressed in virtually all tissues of the body, except for mature skeletal muscle and mobile cell types such as sperm and erythrocytes and provide regulated physical communication between cells by directly linking the interiors of adjoining cells, allowing various molecules, ions and electrical impulses to directly pass through.

One gap junction channel is composed of two connexin hemichannels (also referred to as connexons), which connect across the intercellular space between adjacent cells. Each hemichannel of a gap junction resides in the adjacent cell membrane, and each hemichannel is formed by the covalent oligomerization of six individual connexin (Cx) proteins. See, e.g., Yeager (1998) Structure of cardiac gap junction intercellular channels, J Struct Biol 121: 231-245. Hemichannels can comprise one or more different connexin proteins but are usually in the form of homohexamers.

The human connexin family of genes and proteins now numbers 21. They usually weigh between about 25 and 60 kDa and have an average length of 380 amino acids. All connexins share a common structure as a 4-pass transmembrane (TM) protein that includes several domains, namely, a short intracellular N-terminus (NT), an intracellular loop (IL) and a C-terminus (CT) that is also localized in the cytoplasm, plus two extracellular loops (EL1 and EL2) located outside the cell. The cytoplasmic carboxy terminus can vary considerably in length. See, e.g., Unger, et al., Electron cryo-crystallography of a recombinant cardiac gap junction channel, Novartis Found Symp 1999, 219:22-30 and discussion at 31-43; Leith, E, et al., The connexin 43 C-terminus: A tail of many tales. Biochimica et Biophysica Acta 2018, 1860(1):48-64.

Connexin proteins are commonly named according to their molecular weights, e.g. connexin 26 (Cx26) is a connexin protein of 26 kDa, connexin 43 (Cx43) is 43 kDa, etc. The principal structural difference between connexin proteins is the length of the C-terminal cytoplasmic tail, with connexin 26 having almost no tail (16 amino acids), while connexins 43 and 32 have long and intermediate ones (73 and 156 amino acids, respectively).

While inherited or acquired alterations in the structure and function of connexin proteins have been linked with various diseases (e.g., Delmar, M, et al. Connexins and Disease, Cold Spring Harb Perspect Biol 2018, 10:a029348; Laird and Lampe, Cellular mechanisms of connexin-based inherited diseases. Trends in Cell Biology 2022, 32:58-69), research has also associated connexins with assorted conditions and disorders. See, e.g., Willebrords, J, et al., Connexins and their channels in inflammation Crit Rev Biochem Mol Biol. 2016, 51(6): 413-439; Feng, J, Becker, D L, et al., Connexin 43 upregulation in burns promotes burn conversion through spread of apoptotic death signals, Burns 2020, 46(6):1389-1397; McDouall, A, Green, C R, et al., Connexins, Pannexins and Gap Junctions in Perinatal Brain Injury. Biomedicines 2022, 10:1445 (2022). Connexins have been proposed as therapeutics targets for a number of conditions, including spinal cord injury, perinatal brain injury, nervous system diseases (e.g. Alzheimer's disease, Parkinson's disease), cardiac disorders (e.g. myocardial infarction), ocular disorders (e.g. age-related macular degeneration, diabetic macular edema), acute and chronic wounds (e.g. venous leg ulcers, diabetic foot ulcers), ischemia-reperfusion injury, inflammation, burns and cancer. Reviewed in Laird and Lampe, Therapeutic strategies targeting connexins, Nat Rev Drug Discov. 2018 17(12): 905-921; Lampe and Laird, Recent advances in connexin gap junction biology, Faculty Reviews 2022, 27:11-14. See Becker D L, et al., Translating connexin biology into therapeutics. Semin. Cell Dev. Biol 2016, 50:49-58. See also the articles in the “Junctional Proteins” issue of FEBS Letters Volume 588, Issue 8, Pages: i, 1185-1490 (Apr. 17, 2014), including Zhang J, et al. Connexin hemichannel induced vascular leak suggests a new paradigm for cancer therapy (p. 1365-1371) and Martin P E, et al., Connexins: Sensors of epidermal integrity that are therapeutic targets (p. 1304-1314). See also, e.g., Van Campenhout R, et al., Mechanisms Underlying Connexin Hemichannel Activation in Disease. Int J Mol Sci. 22(7):3503 (April 2021) and U.S. Pat. Nos. 10,401,188 and 11,401,516, issued for “Channel Modulators.”

Pannexins are a family of transmembrane channel glycoproteins that include Panx1, Panx2 and Panx3. Pannexins share similar structural features with connexins, consisting of 4 transmembrane domains, 2 extracellular and 1 intracellular loop, along with intracellular N- and C-terminal tails. Pannexin 1 (Panx1) is a ubiquitously expressed protein forming large conductance channels that are central to various distinct inflammation and injury responses. nPanx1 is expressed in many mammalian tissues, while Panx2 and Panx3 expression is more limited. Panx1 channels have been implicated in ATP release, as well as calcium signaling, and keratinocyte and osteoblast differentiation. One major difference between connexin and pannexin channels is that pannexin channels do not form cell-to-cell channels. Pannexin modulators have previously been described. They include pannexin peptidomimetic compounds (e.g., mPanx1), as well as probenecid and other compounds of Formula VI, including analogs and prodrugs thereof, as set forth in U.S. Pat. No. 10,465,188, issued on Nov. 5, 2019, for “Channel Modulators.”

Inflammasomes are large, cytosolic molecular complexes that typically consist of a sensor protein, the adaptor protein apoptosis-associated speck-like protein containing a caspase-recruitment domain (ASC), and the proinflammatory caspase, caspase-1. They control activation of the proteolytic enzyme caspase-1. Caspase-1 in turn regulates the proteolytic maturation of Interleukin-10 and IL-18, as well as a rapid, noxious, inflammatory form of cell death termed pyroptosis. Assembly of inflammasome complexes is dependent on cytosolic sensing of pathogen-associated molecular patterns (PAMPs) that gain access to the cytosol during microbial infection. PAMPs are relatively non-specific, highly conserved, pathogenic molecular structures expressed in pathogens, and their products (including, e.g., lipopolysaccharide (LPS), which is found on the outer cell wall of gram-negative bacteria). Being derived from microorganisms, PAMPs drive inflammation in response to infections. In addition, endogenous danger signals (danger-associated molecular patterns, or DAMPs) released from damaged or dying cells also activate inflammasomes. DAMPs are derived from host cells including tumor cells, dead or dying cells, or products released from cells in response to signals such as hypoxia. DAMPs are a large number of related intracellular proteins or nucleic acids released by necrotic cells at the site of necrosis. Because they are derived from host materials, DAMPs induce what are known as sterile inflammatory responses. DAMPs are often created or exposed in environments of trauma, ischemia, or tissue damage and do not require pathogenic infection and drive pathological inflammation in sterile inflammatory diseases including atherosclerosis, Alzheimer's disease, diabetes and cancer. See Rathinam and Fitzgerald, Inflammasome Complexes: Emerging Mechanisms and Effector Functions. Cell. 2016, 165(4):792-800.

Four key inflammasomes have been well characterized, i.e., NLRP1, NLRP3, NLRC4, and AIM2. They regulate innate immunity by serving as a signaling platform, and activation of these any one of these inflammasomes leads to the processing and secretion of inflammatory cytokines, including IL-1β and IL-18.

The Nod-like receptor protein 3 (NLRP3) inflammasome, is by far the most extensively studied and well-characterized inflammasome. The NLRP3 inflammasome (also sometimes identified as the “nucleotide-binding domain, leucine-rich-containing family, pyrin domain-containing-3” inflammasome) is expressed in various cells of the cardiovascular system, including in cardiomyocytes, endothelial cells, and immune cells. It is a cytosolic multiprotein complex composed of the innate immune receptor protein NLRP3, the adaptor protein apoptosis-associated speck-like protein (ASC), and inflammatory protease caspase-1 (pro-caspase-1). A variety of stimuli can activate the NLRP3 inflammasome. When activated, the NLRP3 protein recruits the adaptor ASC protein and activates pro-caspase-1, resulting in inflammatory cytokine maturation and secretion, which is associated with inflammation and the induction of gasdermin D-dependent pyroptosis.

Like other inflammasomes, the assembled NLRP3 inflammasome facilitates the release of IL-1β and IL-18, which contribute to innate immune defense and homeostatic maintenance. However, aberrant activation of the NLRP3 inflammasome has been linked to the pathogenesis of various inflammatory diseases, including atherosclerosis, ischemic stroke, Alzheimer's disease, diabetes mellitus and inflammatory bowel disease. Recent studies have revealed that NLRP3 inflammasome activation contributes to not only pyroptosis but also other types of cell death, including apoptosis, necroptosis, and ferroptosis, and the NLRP3 inflammasome has emerged as a therapeutic target for inflammatory diseases. Reviewed in Zhang X, et al., Int J Mol Med. 2023, 51(4):35, which discusses candidate inhibitors of the NLRP3 inflammasome including MCC950 (CP-456,773, CRID3), oridonin (an ent-kaurane diterpenoid), OLT1177 (an orally active β-sulfonyl cyanide molecule), INF39, tranilast (a tryptophan metabolite), CY-09, JC124, 3,4-methylenedioxy-β-nitrostyrene (MNS), parthenolide, BOT-4-one, and others.

Countries throughout the world are now experiencing the impact of post-acute infection disorders, including long COVID, long flu and long cold, and a greater fraction of the population suffers from infection-associated fatigue, brain fog and other post-infection impairments, making it important to elucidate means by which to treat and protect against the effects of post-acute infection syndromes. These disorders have already become a major burden on society with and increased incidence of symptoms and effects of post-acute infection syndromes.

Post-acute infection syndromes, including long COVID, long flu, long cold, post-treatment Lyme disease syndrome, myalgic encephalomyelitis/chronic fatigue syndrome (ME/CFS), etc., have remained an unmet medical need. The need for therapeutics for post-acute infection syndromes is unmet need. There is currently no approved medication for long COVID or other post-acute infection syndromes, including those described or referenced herein. Such compounds are described herein.

### BRIEF INTRODUCTION TO THE INVENTIONS

The inventions described and claimed herein have many attributes and embodiments including, but not limited to, those set forth or described or referenced in this brief introduction. It is not intended to be all-inclusive, and the inventions described and claimed herein are not limited to or by the features or embodiments identified in this introduction, which is included for purposes of illustration and outline only and not restriction. Further description and detail is contained in the Detailed Description and claims, all of which, including this introduction, form a part of the patent specification.

SARS-CoV-2 viruses are not unique in their ability to cause post-acute sequelae. Certain acute infections have long been associated with an unexplained chronic disability in a minority of patients. Post-acute infection syndromes represent a substantial healthcare burden, but there is a lack of understanding of the underlying mechanisms, representing a significant blind spot in the field of medicine. Unfortunately, the association between acute infectious diseases and unexplained chronic disability remains understudied, which leads to poor recognition of these conditions in clinical practice. As a result, patients might experience delayed or a complete lack of clinical care, Choutka, J., et at. Unexplained post-acute infection syndromes. Nat Med 28, 911-923 (2022).

The disclosure is based, in part, on the discovery that connexin hemichannel modulators (e.g., connexin 43 hemichannel modulators) can treat, prevent, alleviate and/or reverse one or more signs or symptoms associated with a post-acute infection syndrome, including one or more signs and/or symptoms of long COVID, long flu, post-treatment Lyme disease syndrome, etc.

The disclosure is also based, in part, on the discovery that inflammasome modulators (e.g., NLRP3 inflammasome modulators), can treat, prevent, alleviate and/or reverse one or more signs or symptoms associated with a post-acute infection syndrome, including one or more signs and/or symptoms of long COVID, long flu, post-treatment Lyme disease syndrome, etc.

The disclosure is further based, in part, on the use of connexin hemichannel modulators (e.g., connexin 43 hemichannel modulators) to treat, prevent, alleviate and/or reverse fatigue associated with a post-acute infection syndrome, including long COVID, long flu, post-treatment Lyme disease syndrome, etc. The disclosure is further based, in part, on the use of inflammasome modulators (e.g., NLRP3 inflammasome modulators), to treat, prevent, treat, alleviate and/or reverse fatigue associated with a post-acute infection syndroms, including long COVID, long flu, post-treatment Lyme disease syndrome, etc. In addition, inventions described herein include the use of connexin hemichannel modulators (e.g., connexin 43 hemichannel modulators) and inflammasome modulators (e.g., NLRP3 inflammasome modulators) to prevent, treat, alleviate and/or reverse weakness and post-exertional malaise associated with post-acute infection syndromes, including those described herein. In addition, inventions described herein include the use of connexin hemichannel modulators (e.g., connexin 43 hemichannel modulators) and inflammasome modulators (e.g., NLRP3 inflammasome modulators) to treat, prevent, alleviate and/or reverse arthralgia, ANA (antinuclear antibody) levels indicative of autoimmunity, and/or neuropathy associated with post-acute infection syndromes, including those described herein.

The disclosure is also based, in part, on the use of connexin hemichannel modulators (e.g., connexin 43 hemichannel modulators) to treat, prevent, alleviate and/or reverse brain fog associated with post-acute infection syndromes, including long COVID, long flu, Post-treatment Lyme disease syndrome, etc., as well as associated polyarthralgia. The disclosure is also based, in part, on the use of inflammasome modulators (e.g., NLRP3 inflammasome modulators) to treat, prevent, alleviate and/or reverse brain fog associated with post-acute infection syndromes, including long COVID, long flu, post-treatment Lyme disease syndrome, etc., as well as associated polyarthralgia.

Numbness and tingling (“pins and needles”), sometimes referred to as paresthesia, is also associated with post-acute infection syndromes, including long COVID. The specification also includes disclosure of inventions based on the use of connexin hemichannel modulators (e.g., connexin 43 hemichannel modulators) to treat, prevent, alleviate and/or reverse numbness paresthesia associated with post-acute infection syndromes, including long COVID, long flu, etc. The specification also includes disclosure of inventions based on the use of inflammasome modulators (e.g., NLRP3 inflammasome modulators) to treat, prevent, alleviate and/or reverse paresthesia associated with post-acute infection syndromes, including long COVID, long flu, etc. In addition, inventions described herein include the use of connexin hemichannel modulators (e.g., connexin 43 hemichannel modulators) and inflammasome modulators (e.g., NLRP3 inflammasome modulators) to treat, prevent, alleviate and/or reverse dizziness, vertigo and balance issues associated with post-acute infection syndromes, including those described herein, as well as numbness and loss of sensation, tremors and peripheral neuropathies.

In addition, connexin hemichannel modulators (e.g., connexin 43 hemichannel modulators) and inflammasome modulators (e.g., NLRP3 inflammasome modulators) can also treat, prevent, alleviate and/or reverse other neuropsychiatric symptoms such as poor attention, difficulty thinking, difficulty with executive functioning, difficulty with problem-solving, slowed thoughts, sudden (acute), confusion and short-term and long-term memory loss associated with post-acute infection syndromes, including those described herein (e.g., long COVID), as well as difficulty finding the right words, difficulty communicating verbally, as well as other speech and language issues and hallucinations.

In some embodiments of the inventions, uses, methods, doses, dose regimens, compositions and kits comprising one or more connexin hemichannel modulators (e.g., connexin 43 hemichannel modulators) and/or one or more inflammasome modulators (e.g., NLRP3 inflammasome modulators) are provided to treat, prevent, alleviate and/or reverse one or more signs or symptoms of a post-acute infection syndrome in a subject. In some embodiments, the post-acute infection syndrome is long COVID. In some embodiments, the post-acute infection syndrome is long flu or long cold. In some embodiments, the post-acute infection syndrome is selected from the group consisting of post-treatment Lyme disease syndrome, post-Ebola syndrome, post-Ebola virus disease syndrome, post-dengue fatigue syndrome, post-polio syndrome, post-SARS syndrome, post-acute sequelae of SARS-CoV-2 invention, post chikungunya chronic inflammatory rheumatism, post-chikungunya disease, post-acute COVID-19 syndrome, post-EBV syndrome, post-West Nile virus syndrome, post-Ross River virus syndrome, post-coxsakie B syndrome, post-H1N1/09 influenza syndrome, post-VZV syndrome, Q fever fatigue syndrome and post-Giardia lamblia syndrome.

In some embodiments of the inventions, for example, uses, methods, doses, dose regimens, compositions and kits comprising one or more connexin hemichannel modulators (e.g., connexin 43 hemichannel modulators) and/or one or more inflammasome modulators (e.g., NLRP3 inflammasome modulators) are provided prevent, alleviate and/or reverse systemic symptoms associated with a post-acute infection syndrome, including fatigue, post-exertional malaise, and weakness.

In some embodiments of the inventions, for example, uses, methods, doses, dose regimens, compositions and kits comprising one or more connexin hemichannel modulators (e.g., connexin 43 hemichannel modulators) and/or one or more inflammasome modulators (e.g., NLRP3 inflammasome modulators) are provided prevent, alleviate and/or reverse musculoskeletal symptoms associated with a post-acute infection syndrome, including muscle aches, muscle spasms, and joint pain.

In some embodiments of the inventions, for example, uses, methods, doses, dose regimens, compositions and kits comprising one or more connexin hemichannel modulators (e.g., connexin 43 hemichannel modulators) and/or one or more inflammasome modulators (e.g., NLRP3 inflammasome modulators) are provided prevent, alleviate and/or reverse cardiovascular symptoms associated with a post-acute infection syndrome, including palpitations, tachycardia, and tightness or pain in the chest.

In some embodiments of the inventions, for example, uses, methods, doses, dose regimens, compositions and kits comprising one or more connexin hemichannel modulators (e.g., connexin 43 hemichannel modulators) and/or one or more inflammasome modulators (e.g., NLRP3 inflammasome modulators) are provided prevent, alleviate and/or reverse cardiovascular symptoms associated with a post-acute infection syndrome, including palpitations, tachycardia, and tightness or pain in the chest.
brain fog, numbness, weakness, peripheral neuropathies, dizziness, confusion.

In some embodiments of the inventions, for example, uses, methods, doses, dose regimens, compositions and kits comprising one or more connexin hemichannel modulators (e.g., connexin 43 hemichannel modulators) and/or one or more inflammasome modulators (e.g., NLRP3 inflammasome modulators) are provided to treat, prevent, alleviate and/or reverse one or more signs or symptoms associated with post-acute infection syndromes, including fatigue, brain fog, numbness, neuropsychiatric symptoms weakness, peripheral neuropathies, dizziness, confusion.

In some embodiments of the inventions, for example, uses, methods, doses, dose regimens, compositions and kits comprising one or more connexin hemichannel modulators (e.g., connexin 43 hemichannel modulators) and/or one or more inflammasome modulators (e.g., NLRP3 inflammasome modulators) are provided to prevent, alleviate and/or reverse fatigue, brain fog, numbness, weakness, peripheral neuropathies, dizziness, confusion associated with a post-acute infection syndrome.

In some embodiments of the inventions, for example, uses, methods, doses, dose regimens, compositions and kits are provided prevent, alleviate and/or reverse fatigue, joint pain, joint swelling, and/or abdominal pain or digestive issues associated with a post-acute infection syndrome.

In some embodiments of the inventions, for example, uses, methods, doses, dose regimens, compositions and kits are provided to prevent, alleviate and/or reverse elevated or abnormal levels of one or more autoimmune markers (e.g., C-reactive protein (CRP), antinuclear antibodies (ANA), antinuclear ribonucleoprotein (anti-RNP) antibodies, and anti-dsDNA antibodies. Patients with extended duration long-COVID-19 syndrome exhibit a distinct immunologic phenotype that includes a poorer SARS-CoV-2 antibody response and autoimmunity. Long COVID patients to be treated as described herein may show a dysregulated immune response, consisting of decreased frequency of detectable neutralizing antibodies, decreased anti-spike antibody levels, and higher frequency of positive ANA titers. See García-Abellán J, et al. Immunologic phenotype of patients with long-COVID syndrome of 1-year duration. Front Immunol. 13:920627 (2022). Persistent positive ANA autoreactivity titers are associated with fatigue, dyspnea, and cough severity in COVID patients. See Cho, OH. Significance of antinuclear antibodies in patients with COVID-19. Korean J Intern Med. 38(3):280-281 (2023).

It is an object of the invention to provide methods, doses, dose regimens, compositions, and kits for connexin hemichannel modulation (e.g., connexin hemichannel modulation) for the treatment of a subject as described herein. It is another object of the invention to provide methods, doses, dose regimens, compositions, and kits for inflammasome inhibition (e.g., NLRP3 inflammasome inhibition) for the treatment of a subject as described herein. In some embodiments, the subject is a human.

In some embodiments, connexin hemichannel modulators (e.g., connexin 43 hemichannel modulators) used in methods, compositions and kits of the invention, including to treat or prevent a post-acute infection syndrome, may be referred to as direct connexin hemichannel modulators, including, for example, the compounds of Formula I, including tonabersat and carabersat, and connexin peptidomimetics such as Peptide5, Gap29 and Gap27. In other some embodiments, useful connexin hemichannel modulators (e.g., connexin 43 hemichannel modulators) may be referred to as indirect connexin hemichannel modulators, including, for example, the connexin peptidomimetics XG19, Gap19, CXT 1 to CXT 5, Antp/CXT 1 to Antp/CXT 5, as well as anti-connexin antisense compounds, including, for example, SEQ ID NO:1 and other connexin-modulating molecules, peptidomimetics and sequences described herein or known in the art. In some embodiments, the connexin hemichannel modulators are connexin 43 hemichannel modulators. In some embodiments, the connexin hemichannel modulators (e.g., connexin 43 hemichannel modulators) are used in and useful for methods, compositions and kits of the invention to treat, prevent, reverse or alleviate one or more signs and/or symptoms of a post-acute infection syndrome.

In some embodiments, inflammasome modulators (e.g., NLRP3 inflammasome modulators) useful in methods, compositions and kits of the invention, including to reverse or alleviate one or more signs and/or symptoms of a post-acute infection syndrome (e.g., long COVID, long flu, post-treatment Lyme disease syndrome, etc.), may be referred to as direct or indirect inflammasome modulators. Useful inflammasome modulators that may be referred to as direct inflammasome modulators include, for example, oridonin, MCC950, tranilast and analogues thereof which directly binds to the NACHT domain of NLRP3 and change its conformation; OLT1177 and parthenolide which suppress the ATPase activity of NLRP3; CY-09 which binds to the NACHT domain and inhibits ATPase of NLRP3; BAY11-7082 and VI-16 which block the binding between TXNIP and NLRP3; NIC7 (NLRP3-inhibitory compound), and its derivatives which inhibit NLRP3-mediated activation of caspase 1 along with the secretion of interleukin (IL)-1β, IL-18 and lactate dehydrogenase. Useful inflammasome modulators that may be referred to as indirect inflammasome modulators include, for example, glyburide, 1673-34-0, FC11A-2, β-hydroxybutyrate, etc., and modulators of ATP-induced inflammasome activation such as tonabersat and other small molecule connexin hemichannel modulators, connexin peptidomimetics including for example, Peptide5, XG19, etc. In some embodiments, the inflammasome modulators are NLRP3 inflammasome modulators. In some embodiments, the inflammasome modulators are direct NLRP3 inflammasome modulators. In some embodiments, the inflammasome modulators are indirect NLRP3 inflammasome modulators. In some embodiments, the inflammasome modulators (e.g., NLRP3 inflammasome modulators) are used in and useful for methods, compositions and kits of the invention relating to reversing or alleviating one or more signs or symptoms of a post-acute infection disorder (e.g., long COVID, long flu, post-treatment Lyme disease syndrome, etc.) in a subject, for example a patient, including, for example, a human patient or subject.

In some embodiments, at least one connexin hemichannel blocker (e.g., connexin 43 hemichannel blocker), indirect inflammasome inhibitor and/or direct inflammasome inhibitor is used to reverse or alleviate or improve one or more signs or symptoms of a post-acute infection disorder (e.g., long COVID, long flu, post-treatment Lyme disease syndrome, etc.) in a subject, for example a patient, including, for example, a human patient or subject.

In some embodiments of any of these methods a combination pharmaceutical composition that comprises two or more of an agent that decreases connexin hemichannel activity (e.g., connexin 43 hemichannel activity) and an agent that decreases or suppresses inflammasome activity or activation (e.g., an NLRP3 inflammasome inhibitor) is administered to a subject. In some embodiments, an agent that decreases pannexin channel activity (e.g., pannexin 1 channel activity) may be includes with a pharmaceutical composition comprising either agent that decreases connexin hemichannel activity (e.g., connexin 43 hemichannel activity) or an agent that decreases or suppresses inflammasome activity or activation (e.g., an NLRP3 inflammasome inhibitor) or both.

Modulation of a hemichannel may occur by any means. In some embodiments, for example, modulation may occur by inducing or promoting closure of a hemichannel; by preventing, blocking, inhibiting or decreasing hemichannel opening; by suppressing hemichannel permeability; by suppressing ATP release from hemichannels; and/or by triggering, inducing or promoting cellular internalization of a hemichannel and/or gap junction. Hemichannel modulators include blockers and other compounds that interfere with the passage of molecules through a connexin hemichannel. A hemichannel modulator can block or reduce the release of molecules through a hemichannel to an extracellular space, and/or block or reduce the entry of molecules through a hemichannel into an intracellular space. In some embodiments, hemichannel modulators fully or partially block hemichannel opening. In some embodiments, hemichannel modulators fully or partially block, slow or suppress the leak or the passage of molecules through a hemichannel to or from an extracellular space. In some embodiments, hemichannel modulators are compounds that decrease the open probability of a hemichannel.

In some embodiments of the invention, the connexin hemichannel modulator (e.g., a connexin 43 hemichannel modulator) is a benzoylamino benzopyran. In some embodiments, the benzoylamino benzopyran is a compound according to Formula I. In some embodiments compound according to Formula I is carabersat. In some embodiments compound according to Formula I is tonabersat. In some embodiments, the tonabersat compound is a tonabersat prodrug. In some embodiments the tonabersat prodrug is a compound according to Formula II.

In some embodiments, the connexin modulator comprises a connexin peptidomimetic. In some embodiments, the connexin peptidomimetic is a connexin43 peptidomimetic. In some embodiments, the connexin 43 hemichannel modulator peptidomimetic molecule is, comprises, or consists essentially of, for example, Peptide5, Gap19, XG19, CXT 1 to CXT 5, Antp/CXT 1 to Antp/CXT 5, etc., or a compounds selected from Table II (SEQ ID NOS: 101-102, 104, 107-108, 112-117, 119-123, 128-129, 140-207). In some embodiments, the connexin peptidomimetic is a connexin 37, connexin 40 and/or connexin 45 peptidomimetic.

In some embodiments, the connexin peptidomimetic comprises, consists essentially of, or consists of SRPTEKT (SEQ ID NO:101). In some embodiments, the connexin peptidomimetic comprises, consists essentially of, or consists of Gap19, XG19, Gap26 or Gap27. In some embodiments, the connexin peptidomimetic is a connexin26 peptidomimetic (e.g. Gap26 i.e., VCYDKSFPISHVR (SEQ ID NO:102), a connexin32 peptidomimetic (e.g. INCTLQPGCNSV (SEQ ID NO: 103) or ^{37,43}Gap27, which is SRPTEKTIFII (SEQ ID NO: 104) or a connexin45 peptidomimetic, or a connexin50 peptidomimetic (e.g. TAT-Cx50L2, i.e., GGERAPLAADQGSVKKSSSSSKGTKK (SEQ ID NO: 105) or TAT-Cx50CT, i.e., SRARSDDLTV (SEQ ID NO: 106)).

In some embodiments the connexin hemichannel modulator modulates connexin expression. In some embodiments, the connexin expression modulator modulates connexin 43 expression. In other embodiments, the connexin expression modulator modulates expression of a vascular connexin. In other embodiments, the connexin expression modulator modulates expression of connexin 37, connexin 40 and/or connexin 45. In some embodiments, the connexin hemichannel modulator (e.g., a connexin 43 hemichannel modulator) is a connexin (e.g., a connexin 43) antisense molecule. In some embodiments, the connexin modulator may be a connexin 43 antisense polynucleotide comprising, consisting essentially of, or consisting of a sequence according to SEQ ID NOS.1-16 and/or modified versions thereof. In some embodiments, antisense oligonucleotide comprises, consists essentially of, or consists of 5′-GTA ATT GCG GCA AGA AGA ATT GTT TCT GTC-3′ (SEQ ID NO:1). In some embodiments, the antisense oligonucleotide may be chemically modified or may be an unmodified oligonucleotide, e.g. a modified or an unmodified DNA oligonucleotide.

In some embodiments, the connexin hemichannel modulator (e.g., a connexin 43 hemichannel modulator) is an anti-connexin (e.g., an anti-connexin 43) antibody (e.g., an anti-connexin monoclonal antibody), an anti-connexin (e.g., an anti-connexin 43) antibody fragment (e.g., an anti-connexin Fab, F(ab′)₂, etc.), an anti-connexin (e.g., an anti-connexin 43) engineered antibody (e.g., an anti-connexin single-chain variable (scFv) fragment or scFab, etc.), an anti-connexin (e.g., an anti-connexin 43) Ig domain (e.g., an anti-connexin 43 VHH), an anti-connexin nanobody (Nb), etc. In other embodiments, one or more of these connexin modulators is directed to a vascular connexin. In other embodiments, one or more of these connexin modulators is directed against connexin 37, connexin 40 and/or connexin 45.

In some embodiments, the connexin hemichannel modulators used in methods of the invention modulates one or more of connexin 26 or a connexin 26 hemichannel, connexin 30 or a connexin 30 hemichannel, connexin 30.3 or a connexin 30.3 hemichannel, connexin 31 or a connexin 31 hemichannel, connexin 31.1 or a connexin 31,1 hemichannel, connexin 32 or a connexin 32 hemichannel, connexin 50 or a connexin 50 hemichannel and connexin 58 or a connexin 58 hemichannel.

Other agents useful alone, or together with connexin hemichannel modulators and/or inflammasome modulators in compositions, methods, kits, doses and dose regimens of the invention comprise pannexin channel modulators. In some embodiments, the pannexin channel modulator is a pannexin 1 channel modulator. In some embodiments, the pannexin 1 channel modulator is probenecid. In some embodiments, the pannexin 1 channel modulator an analog or prodrug of probenecid. In some embodiments, the pannexin 1 channel modulator is a compound according to Formula VI in U.S. Pat. No. 10,465,188, or an analog or prodrug thereof. In some embodiments, the pannexin 1 channel modulator is a mimetic peptide blocker of pannexin 1 a (e.g., ¹⁰Panx1, or an analogue of thereof).

Caspases are a family of intracellular cysteine proteases that cleave a limited number of substrates after aspartic acid residues. The function of caspases has been extensively studied in apoptosis where they play an essential role in the activation and implementation of cellular demise. However, several caspases including the human caspase-1, -4, and -5 are involved in the processing and secretion of pro-inflammatory molecules and are often refereed as “pro-inflammatory caspases. The inflammasome is a multi-protein complex that mediates activation of caspase-1 which promotes the secretion of the proinflammatory cytokines IL-1β and IL-18 as well as pyroptosis, a form of cell death induced by bacterial pathogens. In some embodiments, the inflammasome modulator used to treat, prevent or ameliorate one or more signs or symptoms of a post-acute infection syndrome, disease, disorder or conditions is a compound that modulates caspsase-1 activation or activity. In some embodiments that inflammasome pathway modulator that targets caspase-1 is belnacasan (VX-765).

NOD-like receptor (NLR), family pyrin domain containing 3 (NLRP3) assembles a protein complex known as the NLRP3 inflammasome upon sensing certain pathogen products or sterile danger signals. This inflammasome processes pro-interleukin-1 β (pro-IL-1β) and pro-IL-18 into bioactive IL-1β and IL-18, respectively, and cleaves gasdermin) (GSDMD). GSDMD amino-terminal fragments form plasma membrane pores that facilitate the secretion of IL-1β and IL-18 and lead to the inflammatory cell death pyroptosis. Accordingly, GSDMD inactivation results in negligible spontaneous inflammation in various experimental models such as in Nlrp3^{CA/+} mice lacking GSDMD (Nlrp3^{CA/+}; Gsdmd^{-/-} mice). In some embodiments, the inflammasome modulator used to treat, prevent or ameliorate one or more signs or symptoms of a post-acute infection syndrome, disease, disorder or conditions is a compound that modulates GSDMD activation or activity. In some embodiments that inflammasome pathway modulator that targets GSDMD is selected from the group consisting of necrosulfonamide, disulfiram and dimethyl fumarte.

In some embodiments, a compound or composition for modulation or inhibition of a connexin hemichannel and/or an inflammasome is provided for treatment and is dosed as described herein.

In some embodiments of the invention, a connexin 43 hemichannel is modulated or inhibited. In some embodiments, a connexin 43 hemichannel is modulated or inhibited in order to reduce the release of ATP from the hemichannel.

In some embodiments, a compound or composition for modulation or inhibition of an inflammasome is provided for treatment and is dosed as described herein. In some embodiments, a compound or composition for modulation or inhibition of an NLRP3 inflammasomes is provided for treatment and is dosed as described herein.

In some embodiments, a pannexin 1 channel is modulated or inhibited. In some embodiments, a pannexin 1 channel is modulated or inhibited in order to reduce ATP release from a pannexin channels.

In some embodiments, the cell or cell population treated using a compound, composition or method of the invention comprises a human cell or a human cell population. In some embodiments, the tissue treated using a compound, composition or method of the invention is a human tissue. In some embodiments, an organ treated using a compound, composition or method of the invention is a human organ. In some embodiments, the subject treated using a compound, composition or method of the invention is a human.

In some embodiments, the connexin hemichannel modulator or the inflammasome inhibitor is orally, systemically, topically, or parenterally. In some embodiments, administration is local. In some embodiments, the connexin hemichannel modulator or the inflammasome inhibitor is administered to the skin. In some embodiments, the connexin hemichannel modulator or the inflammasome inhibitor is administered transdermally. In some embodiments, the connexin hemichannel modulator or the inflammasome inhibitor is administered to the eye. In some embodiments, the connexin hemichannel modulator or the inflammasome inhibitor is administered topically to the eye.

In some embodiments of the present invention for topical administration, e.g. to the skin, to the eye, or to an internal organ, the method comprises administering to the subject a therapeutically effective amount of composition comprising an agent (e.g., a connexin or connexin hemichannel modulator), wherein the composition comprises a compound selected from the group consisting of reverse thermosetting gels, poloxamer gels, nonionic polyoxyethylene-polyoxypropylene block co-polymers (e.g. Pluronic F-127) and cellulose-based carriers (e.g., hydroxyethylcellulose, carboxymethylcellulose hydroxymethylcellulose, hydroxypropylmethylcellulose, etc.) and other pharmaceutically acceptable carriers. In some embodiments, the agent is selected from the group consisting of small molecule, antisense, peptidomimetic, antibody and antibody fragment connexin hemichannel modulators (e.g., a connexin 43 hemichannel modulator). In some embodiments, the connexin 43 hemichannel modulator modulates connexin 43 hemichannel activity (e.g., ATP release). In some embodiments, the connexin 43 hemichannel modulator modulates connexin 43 protein expression. In some embodiments, the connexin 43 protein expression modulator is an antisense molecule. In some embodiments, the connexin 43 antisense molecule is SEQ ID NO:1.

In some embodiments, the topical composition for use in the methods of the invention comprises poloxamer F-127 (poloxamer 407 or Pluronic F-127). In some embodiments, the composition for use in the methods of the invention comprises about 20-30 mg/mL of a poloxamer F-127. In some embodiments, the composition for use in the methods of the invention comprises about 22-25 mg/mL of a poloxamer F-127. In some embodiments, the composition for use in the methods of the invention comprises about 22.6 mg/mL of a poloxamer F-127.

In some embodiments, a combination of two or more compounds selected from the group consisting of connexin hemichannel modulators (e.g. connexin hemichannel modulators), pannexin channel modulators (e.g. pannexin 1 channel modulators), inflammasome modulators (e.g. NLRP3 inflammasome modulators) are administered to a cell, to a cell population, to a tissue, to an organ, or to a subject.

In some embodiments, the connexin hemichannel modulator is administered to a subject using any of the dosing schedules described herein in a therapeutically effective amount.

In some embodiments, the amount of connexin modulator administered to a subject in any of the dosing schedules described herein is one or more of the dose amounts described herein (e.g. milligram per milliliter (mg/mL) connexin modulator dosing, micromolar (μM) connexin modulator concentration dosing, milligram (mg) connexin modulator dosing, etc.).

In some embodiments, the invention provides the use of a connexin modulator in the manufacture of a medicament for treatment of any of the diseases, disorders or conditions described herein. In some embodiments, the medicament comprises, consists essentially of, or consists of, an agent as described herein, including one or more connexin expression or connexin hemichannel modulators (e.g., connexin 43 hemichannel modulators) and/or including one or more inflammasome modulators (e.g., NLRP3 inflammasome modulators). In some embodiments, the agent is, comprises, or consists essentially of, a molecule according to Formula I (e.g., tonabersat or carabersat), a tonabersat prodrug (e.g., a molecule according to Formula II), a connexin peptide mimetic (e.g., XG19, Peptide5, Gap19, an aCT1 peptide (e.g., CXT 1, CXT, CXT 3, CXT 4, CXT 5, or any other C-terminal connexin peptidomimetic, with or without a C-terminally or N-terminally attached cell-penetrating peptide), etc.), and/or a connexin antisense molecule (e.g., SEQ ID NO:1), including in the amounts and/or concentrations described herein.

In some embodiments, the invention relates to pharmaceutical compositions and articles of manufacture, including kits comprising a therapeutically effective amount of a connexin hemichannel modulator (e.g., a connexin 43 hemichannel modulator) and/or an inflammasome inhibitor (e.g., an NLRP3 inflammasome inhibitor), and dose regimens for treating one or more of the diseases, disorders or conditions described herein, including, for example, treatment of one or more signs and/or symptoms of a post-acute infection syndrome (e.g., long COVID, long flu, post-treatment Lyme disease syndrome, etc.). In some embodiments, the kit includes or is associated with instructions (e.g. included instructions, online instructions, QR code instructions, etc.) for use. The instructions may provide instructions for dosing and treating diseases, disorders and conditions described or referred to herein. In some embodiments, the kit includes means for administration of the connexin hemichannel modulator(s) and/or the inflammasome inhibitor(s).

In some embodiments, a connexin modulator(s) (e.g. a connexin expression modulator, a connexin gap junction modulators, a connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) are combined with a pharmaceutically acceptable carrier or diluent to produce a pharmaceutical composition. In some embodiments, suitable carriers and diluents include buffered, aqueous solutions, isotonic saline solutions, for example phosphate-buffered saline, isotonic water, and the like. In some embodiments, the carrier is a pluronic or poloxamer gel. In some embodiments, the gel is pluronic F-127. In some embodiments, the pharmaceutical composition comprises a buffer. In some embodiments, the buffer comprises or consists essentially of sodium phosphate dibasic heptahydrate or sodium phosphate monobasic or both.

In some embodiments, methods of the invention, the doses and the dose regimens, and the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) and/or inflammasome inhibitor(s) (e.g., an NLRP3 inflammasome inhibitor(s)), described herein are used for the treatment of a subject for one or more of the diseases, disorders or conditions described or referred to herein alone or in combination with or in conjunction with another therapeutic agent.

In some embodiments, doses of the connexin, inflammasome, and/or pannexin modulators described or referenced herein are administered with, or co-formulated for administration to a subject with, effective amounts of one or more compounds selected from the group consisting of anti-microbial agents, antibiotics, antivirals, anti-fungals, anti-parasitics, anti-oxidants, statins and other cholesterol drugs, ACE inhibitors (e.g. ramipril), angiotensin-II antagonists (e.g., losartan), anti-arrhythmic medicines (e.g., amiodarone), anticoagulant medicines (e.g., dabigatran), anti-platelet medicines (e.g., clopidogrel), beta-blockers (e.g., bisoprolol), calcium-channel blockers (e.g., amlodipine), steroidal anti-inflammatories, immunosuppressants, and immunomodulatory agents.

In some embodiments of any of these methods a combination pharmaceutical composition that comprises two or more of an agent that decreases connexin hemichannel activity (e.g., connexin 43 hemichannel activity) and/or an agent that decreases or suppresses inflammasome activity or activation (e.g., an NLRP3 inflammasome inhibitor) is administered to a subject alone or in combination with one or more of an antiviral agent (e.g., an agent comprising paxlovid, remdesivir, etc.), an anti-inflammatory agent (e.g., an agent comprising baricitinib, tocilizumab, etc.), a steroid or corticosteroid agent (e.g., an agent comprising a cortison, a prednisone, a prednisolone, a methylprednisolone, a dexamethasone, a betamethasone, a hydrocortisone, etc.), an immune suppression agent (e.g., an agent comprising anakinra, etc.), a complement inhibitor (e.g., an agent comprising empavelia, etc.), other immunosuppressants (e.g., an agent comprising a TNF inhibitor, including Enbrel, Humira, etc.), an antibody agent (e.g., an agent comprising vilobelimab), and other agents including, for example, ivermectin, hydroxychloroquine and chloroquine. Antiviral, anti-inflammatory, steroid, corticosteroid, immune suppression, antibody, and other drugs and agents useful in the treatment of one or more signs or symptoms of a post-acute infection disorder may be administered to a post-acute infection syndrome patient as a combination pharmaceutical in combination with a connexin hemichannel modulator (e.g., a connexin 43 hemichannel modulator/inhibitor), an inflammasome modulator (e.g., an NLRP3 inflammasome modulator/inhibitor), and/or a pannexin channel modulator (e.g., a Panx 1 channel modulator/inhibitor) or they may be administered separately at or about the same time as a connexin hemichannel modulator (e.g., a connexin 43 hemichannel modulator/inhibitor), an inflammasome modulator (e.g., an NLRP3 inflammasome modulator/inhibitor) and/or a pannexin channel modulator (e.g., a Panx 1 channel modulator/inhibitor). In some embodiments, doses of one or more of the connexin, inflammasome, and/or pannexin modulators described or referenced herein are co-formulated for administration to a subject with effective amounts of one or more these agents. In some embodiments, doses of one or more of the connexin, inflammasome, and/or pannexin modulators described or referenced herein are administered to a subject with an effective amount of one or more these agents.

In another aspect the present disclosure provides a method of selecting or quantifying the activity of a candidate compound for the treatment of a post-acute infection disorder. In some embodiments, the method comprises providing a first isolated cell or preparation of cells or tissue; determining ATP levels in the cell or cell preparation or tissue; comparing ATP levels in the cell or cell preparation or tissue to a reference level; contacting it with a test compound (i.e., one or more candidate agents) to the cell or the cell preparation or tissue, or to another to preparation of like or similar cells or tissue(s), and remeasuring ATP levels in the cell or preparation of cells or tissue. The candidate test compound may then be evaluated for dosing in and animal model of a post-acute infection disorder, and for the treatment or prevention of one or more signs or symptoms thereof. In some embodiments, the test compound is a connexin hemichannel modulator (e.g., a connexin 43 hemichannel modulator). In some embodiments, the test compound is an inflammasome inhibitor or candidate inhibitor (e.g. an NLRP3 inflammasome inhibitor or candidate NLRP3 inflammasome inhibitor). In other embodiments, the test compound may be a pannexin channel modulator (e.g., a pannexin 1 channel modulator). In some embodiments, the test compound is a small molecule. In some embodiments, the test compound is a small molecule that modulates, may modulate, or is suspected of modulating a connexin hemichannel (e.g., a connexin 43 hemichannel). In some embodiments, the test compound is a small molecule that modulates, may modulate, or is suspected of modulating an inflammasome or inflammasome activity or activation (e.g., an NLRP3 inflammasome inhibitor or NLRP3 inflammasome activity or activation). In some embodiments, the test compound may be a small molecule that modulates, may modulate, or is suspected of modulating release of ATP from a pannexin channel (e.g., a pannexin 1 channel). In some embodiments the cells or cell preparation are human. In some embodiments the cells or cell preparation are rodent. In some embodiments the cells or cell preparation are from a non-human primate.

In some embodiments, a test compound is administered in a high dose. In some embodiments, a test compound is administered a cell isolated from a patient having a disease, disorder or condition. In some embodiments, the test compound is administered to a stem cell, e.g., an iPS cell. In some embodiments, the test compound is administered to a stem cell, e.g., an iPS, cell derived from a patient (e.g., a patient's skin or other tissue). In some embodiments, the cell is an isolated human cell. In some embodiments, the cell is derived from a patient's skin or other tissue. In some embodiments, the isolated human cell is derived from a patient's bone marrow. In some embodiments, the cell is an isolated human cell and is derived from a patient's blood.

In some embodiments, the cell used to contact a test compound or other candidate compound or agent is an isolated human cell that is derived from a patient having a post-acute infection syndrome (e.g., a post-acute infection syndrome referenced or described herein). In some embodiments, the patient is suspected of having post-acute infection syndrome. In some embodiments, the patient has symptoms of a post-acute infection syndrome. In some embodiments, the patient is diagnosed as having a post-acute infection syndrome.

In some embodiments of this test/screening method, tonabersat, Peptide5 or XG19 is used as a positive control.

In some embodiments, the invention provides methods for treating a subject or patient for a post-acute infection syndrome (actively or prophylactically or for maintenance) comprising administering to the subject or patient an effective amount of at least one connexin hemichannel modulator (e.g., a connexin 43 hemichannel modulator) and/or at least one inflammasome modulator (e.g., an NLRP3 inflammasome modulator). Administration to the subject or patient of an effective amount of a connexin hemichannel modulator and/or an inflammasome modulator or pannexin channel modulator lessens, slows, halts, or substantially halts one or more signs or symptoms of a post-acute infection syndrome in the subject or patient.

In some embodiments, the subject or patient having or suspected of having a post-acute infection syndrome has one or more of fatigue, brain fog, trouble concentrating, peripheral neuropathy, muscle pain and/or joint pain that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator).

In some embodiments, the subject or patient having or suspected of having a post-acute infection syndrome has one or more of fatigue, brain fog, trouble concentrating, peripheral neuropathy, muscle pain and/or joint pain that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator).

In some embodiments, the uses, methods, doses, dose regimens, compositions and kits prevent, alleviate and/or reverse the presence of one or more elevated autoimmune markers. Autoimmune markers include, for example, those selected from the group consisting of elevated C-reactive protein (CRP), antinuclear antibodies (ANA), antinuclear ribonucleoprotein (anti-RNP) antibodies, anti-dsDNA antibodies, and erythrocyte sedimentation rate (ESR), amongst others.

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising a brain or neurological sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the brain or neurological sign or symptom is selected from the group consisting of brain fog, peripheral neuropathies (pins & needles/numbness any body part), dizziness, confusion, hallucinations, loss of smell and/or taste, phantom smells and/or taste, tinnitus (ringing in ears), internal vibrations/tremors, speech issues/stutter, stroke, seizures, and headaches.

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising an ocular sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the ocular sign or symptom is selected from the group consisting of eye pain, burning eye pain, feeling of sand/glass in eyes, eye redness, vision changes, visual disturbances/floaters.

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising a breathing or respiratory sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the breathing or respiratory sign or symptom is selected from the group consisting of shortness of breath, cough, post exertional malaise (tired after minor exertion), and fatigue or extreme fatigue/exhaustion without exertion.

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising a digestive or gastrointestinal sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the digestive or gastrointestinal sign or symptom is selected from the group consisting of nausea, vomiting, diarrhea, bloating, and early satiety (feeling full after eating small amounts of food).

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising a cardiovascular sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the cardiovascular sign or symptom is selected from the group consisting of heart arrythmias, tachycardia, bradycardia and chest pain.

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising a musculoskeletal sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the musculoskeletal sign or symptom is selected from the group consisting of joint pain, muscle pain and bone pain.

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising a dermatological sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the dermatological sign or symptom is selected from the group consisting of skin rashes and hair loss.

In some embodiments, the subject or patient having or suspected of having a post-acute infection syndrome (e.g., long COVID, long flu, post-treatment Lyme disease syndrome, etc.) has one or more signs or symptoms of a post-acute infection disorder comprising a mental health sign or symptom that is/are lessened, slowed, halted, substantially halted, alleviated or improved by administration of a connexin hemichannel modulator (e.g., a connexin 43 hemichannel modulator) and/or an inflammasome modulator (e.g., an NLRP3 inflammasome modulator) or pannexin channel modulator (e.g., a Panx 1 channel modulator). In some embodiments, the mental health sign or symptom is selected from the group consisting of anxiety and depression.

In some embodiments, the subject or patient having or suspected of having a post-acute infection syndrome (e.g., long COVID, long flu, post-treatment Lyme disease syndrome, etc.) has one or more signs or symptoms of a post-acute infection disorder selected from the group consisting of flushing of the face, facial paralysis, ptosis, bruising, cold hands and/or feet, heat and/or cold intolerance, feeling of fever without having one, and mottling of hands and/or feet.

In some embodiments of the methods of the invention, the inflammasome modulator is a direct inflammasome modulator. In some embodiments of the methods of the invention, the inflammasome modulator is an indirect inflammasome modulator. In some embodiments of the methods of the invention, the inflammasome modulator is an NLRP3 inflammasome modulator. In some embodiments of the methods of the invention, the inflammasome modulator is a connexin hemichannel modulator. In some embodiments of the methods of the invention, the NLRP3 inflammasome modulator is a connexin 43 hemichannel modulator.

In some embodiments of the methods of the invention, the connexin hemichannel modulator is a compound according to Formula I. In some embodiments of the methods of the invention, the connexin hemichannel modulator is a compound according to Formula II. In some embodiments of the methods of the invention, the connexin hemichannel modulator is tonabersat or carabersat. In some embodiments of the methods of the invention, the connexin hemichannel modulator is tonabersat prodrug or a carabersat prodrug. In some embodiments of the methods of the invention, the connexin hemichannel modulator is connexin peptidomimetic. In some embodiments, the peptidomimetic connexin hemichannel modulator is a connexin 43 peptidomimetic. In some embodiments, the peptidomimetic connexin hemichannel modulator is XG19, Peptide5, Gap19, Gap20, Gap22, or any of the alpha CT peptides. In some embodiments, the peptidomimetic connexin hemichannel modulator is another peptidomimetic connexin hemichannel modulator described herein or known in the art. In some embodiments of the methods of the invention, the connexin hemichannel modulator is connexin antisense compound. In some embodiments, the connexin antisense compound is SEQ ID NO:1. In some embodiments, the connexin antisense compound is another connexin antisense compound described herein or known in the art. In some embodiments the connexin hemichannel modulator is a connexin binding compound (e.g., an antibody, antibody fragment, ScFv, etc.)

In some embodiments of the methods of the invention, the inflammasome modulator is a pannexin channel modulator. In some embodiments, the pannexin channel modulator is an NLRP3 inflammasome modulator. In some embodiments, the pannexin channel modulator is a pannexin 1 channel modulator. In some embodiments, the pannexin channel modulator compound is a compound according to Formula III. In some embodiments, the pannexin channel modulator compound is probenecid. In some embodiments of the methods described herein, an effective amount of a pharmaceutical composition comprising an agent that decreases pannexin channel activity (e.g., pannexin 1 channel activity including release of ATP) is administered to a cell, subject or patient.

In some embodiments of the methods of the invention, a therapeutically or prophylactically effective amount of a connexin hemichannel modulator (e.g., a connexin 43 hemichannel modulator), an inflammasome modulator (e.g., an NLRP3 inflammasome modulator), and/or a pannexin channel 1 modulator (e.g., a pannexin channel modulator) is/are administered to a subject or patient to protect against the occurrence of one or more signs and/or symptoms, or an increase one or more signs and/or symptoms, of a post-acute infection syndrome (e.g., long COVID, long flu, post-treatment Lyme disease syndrome, etc.).

In some embodiments of the methods of the invention, a therapeutically or prophylactically effective amount of a connexin hemichannel modulator (e.g., a connexin 43 hemichannel modulator), an inflammasome modulator (e.g., an NLRP3 inflammasome modulator), and/or a pannexin channel 1 modulator (e.g., a pannexin channel modulator) is/are administered to a subject or patient at least once per day.

In some embodiments of the methods of the invention, a therapeutically or prophylactically effective amount of a connexin hemichannel modulator (e.g., a connexin 43 hemichannel modulator), an inflammasome modulator (e.g., an NLRP3 inflammasome modulator), and/or a pannexin channel 1 modulator (e.g., a pannexin channel modulator) is/are administered orally, topically, or parenterally to a subject or patient.

In some embodiments of the methods of the invention, a therapeutically or prophylactically effective amount of a connexin hemichannel modulator (e.g., a connexin 43 hemichannel modulator), an inflammasome modulator (e.g., an NLRP3 inflammasome modulator), and/or a pannexin channel 1 modulator (e.g., a pannexin channel modulator) is/are administered orally, topically, or parenterally to a subject or patient.

In some embodiments of the methods of the invention, about 80 to about 400 milligrams per day of a connexin hemichannel modulator (e.g., a connexin 43 hemichannel modulator), is administered to a subject or patient. In some embodiments, the connexin hemichannel modulator is administered in single or divided doses that are administered one or more times per day or per week. In some embodiments, the connexin hemichannel modulator is a compound according to Formula I. In some embodiments, connexin hemichannel modulator is tonabersat. In some embodiments, connexin hemichannel modulator is a tonabersat prodrug. In some embodiments, the tonabersat prodrug is a compound according to Formula II. In other embodiments of the methods of the invention, the connexin hemichannel modulator (e.g., a connexin 43 hemichannel modulator), such as, for example, a compound according to Formula I (e.g., tonabersat or carabersat) or a compound according to Formula II is administered to a subject in need thereof in amounts including at least about 40 milligrams per dose, at least about 50 milligrams per dose, at least about 60 milligrams per dose, at least about 70 milligrams per dose, at least about 80 milligrams per dose, at least about 90 milligrams per dose, at least about 100 milligrams per dose, from about 40 to about 400 or about 500 milligrams per dose. In some embodiments, the connexin hemichannel modulator dose is administered in single or divided doses and the dose may be administered from 1-4 times daily, 1-4 times every other day, 1-4 times every third day, 1-4 times twice a week or 1-4 times once per week. Antisense and peptidomimetic connexin hemichannel modulator doses include these doses and others described herein.

In some embodiments, at least about 40 milligrams of tonabersat is administered to a subject or patient. In some embodiments, at least about 80 milligrams of tonabersat, or another compound according to Formula I or according to Formula II, is administered to a subject or patient. In some embodiments, at least about 100 to about 400 milligrams per day of tonabersat, or another compound according to Formula I or according to Formula II, is administered to a subject or patient.

In some embodiments of the methods of the invention, about 2 5 to about 600 milligrams per day of an inflammasome modulator (e.g., an NLRP3 inflammasome modulator) is administered to a subject or patient.

In some embodiments of the methods of the invention, about 40 to about 500 milligrams per day of a pannexin channel 1 modulator (e.g., a pannexin channel modulator) is administered to a subject or patient.

In some embodiments of the methods of the invention, the subject or patient is a human subject or a human patient.

### DETAILED INVENTION DISCLOSURES

Post-acute infection syndrome refers to chronic diseases that occur after an acute viral, bacterial or parasitic infection. While not new, the syndrome's prevalence is significantly growing as a result of the COVID-19 pandemic, in which as many as 10% of infections have led to Long COVID, leaving individuals with lingering, often debilitating symptoms. Some researchers estimate that 75 million people globally are suffering with the disease, and as new infections continue to arise daily.

The inventions described in this patent relate to compounds and compositions for treating and/or preventing post-acute infection syndromes, including long-term sequalae of coronavirus infection and signs and symptoms of long-haul effects resulting from other infections, including but not limited to infections by viruses that cause the “flu” (e.g., Haemophilus influenzae and other influenza viruses including the influenza A or B virus and the parainfluenza virus including parainfluenza virus Type 1, Type 2 and Type 3), microbial infections that cause the common “cold” (e.g., rhinoviruses), infections causing pneumonia (e.g., Chlamydia pneumoniae, Streptococcus pneumoniae, Mycoplasma pneumoniae, and others), infections causing the common “cold” (e.g., rhinoviruses), and infections caused by respiratory syncytial viruses, adenoviruses, enteroviruses, and bacteria such as Moraxella catarrhalis (also known as Neisseria catarrhalis and Branhamella catarrhalis) and others which can also cause pneumonia and middle ear infections, sinus infections, bronchitis, and COPD (a group of lung diseases that can worsen over time and include chronic bronchitis, emphysema, and refractory asthma), for example, as well as the bacterium Borrelia burdorferi (and, more rarely, Borrelia mayonii) which causes Lyme disease and can lead to post-treatment Lyme disease syndrome. About ten percent of the Lyme disease patients have prolonged subjective symptoms of fatigue, cognitive impairment, and/or musculoskeletal pain after antibiotic therapy and are referred to as post-treatment Lyme disease syndrome (PTLDS). The underlying causes, treatment, and epidemiology of PTLDS have remained elusive. Myalgic encephalomyelitis/chronic fatigue syndrome (ME/CFS) is a neuroinflammatory, neuroimmune illness most commonly triggered by infection, such as Epstein-Barr virus, and is a post-infection syndrome, the signs and symptoms of which may be treated as described herein using the compounds and compositions described and referenced here in. Some ME/CFS patients have reported other triggers as well, such as physical accidents and environmental exposures, and they may also be treated as described herein. Post-acute infection syndromes may be diagnosed as described in the art. The signs and symptoms of post-acute infection syndromes are well known.

Many post-acute infection syndromes have been called “fatigue syndromes,” although this term is likely too narrow to encompass the more complex clinical picture typically seen in such disorders. Although the clinical presentation of a post-acute infection syndrome is heterogeneous, often including long and varied symptom lists, there is significant overlap, and several characteristic symptom clusters can be identified as a common denominator. The prime manifestations include an overall poor functional status, exertion intolerance, debilitating fatigue, and unrefreshing sleep. Other characteristic features include neurocognitive and sensory impairments, dysautonomia, musculoskeletal complaints, flu-like symptoms, and other feelings of illness. Irritability, mood swings, and signs of depression, as well as a wide range of other nonspecific neurological and immunological symptoms (Box 2), are frequently present.

The terms post-infectious fatigue syndrome and post-viral fatigue syndrome are sometimes used to describe debilitating fatigue following an infection, often accompanied by other signs and symptoms. Post-infectious and post-viral fatigue syndromes were originally postulated as subsets of ‘chronic fatigue syndrome,’ in which the triggering infectious agent is objectively documented67. However, there appears to be no clear consensus at present about the distinctions across these concepts. It is thus unclear whether these terms should be considered synonymous to the ME/CFS label, any of its subsets, or include a wider range of post-infectious fatigue conditions.

Long-haul effects of infections in an organism carry significant risks of disability and disease. Some show a greater risk across multiple organ systems (e.g., COVID) while others which may be more limited, for example those with ongoing health risks found mostly in the respiratory system (e.g., influenza).

Long COVID or post-COVID-19 syndrome first gained widespread recognition among social support groups and later in the scientific and medical communities. This illness is understood to affect COVID-19 survivors at all levels of disease severity, even younger adults, children, and those not hospitalized. See, e.g., Yong S J. Long COVID or post-COVID-19 syndrome: putative pathophysiology, risk factors, and treatments. Infect Dis (Lond). 53(10):737-754 (2021).

While a precise definition of long COVID was initially lacking, the World Health Organization now defines long COVID as “the continuation or development of new symptom 3 months after the initial SARS-CoV-2 infection, with these symptoms lasting for a least 2 months with no other explanation.” See also Thaweethai T, et al. Development of a Definition of Postacute Sequelae of SARS-CoV-2 Infection. JAMA 329(22):1934-1946 (2023). The most common symptoms reported in many studies are fatigue, brain fog and dyspnea that last for months after acute COVID-19. Other persistent symptoms may include other chest and joint pains, palpitations, myalgia, smell and taste dysfunctions, cough, headache, and gastrointestinal and cardiac issues. See Davis, H E, et al., Characterizing long COV ID in an international cohort: 7 months of symptoms and their impact E Clincial Medicine 38:101019 (2021).

Presently, there is limited literature discussing the possible pathophysiology, risk factors, and treatments in long COVID. In brief, long COVID may be driven by long-term tissue damage (e.g. lung, brain, and heart) and pathological inflammation (e.g. from viral persistence, immune dysregulation, and autoimmunity).

A recent study comparing the viruses that cause COVID-19 and the flu revealed that in the 18 months after infection, patients hospitalized for either COVID-19 or seasonal influenza faced an increased risk of death, hospital readmission, and health problems in many organ systems. Xie, Y, et al., Long-term outcomes following hospital admission for COVID-19 versus seasonal influenza: a cohort study Lancet Infect Dis 24: 239-255 (2024).

Post-acute infection syndrome encompasses chronic illnesses that occur after an infection from a virus, bacteria, or parasite. This includes prolonged symptoms after COVID, caused by the SARS-CoV-2 virus, as well as other infections caused by viruses, bacteria, or parasites, or Lyme disease, which is caused by certain types of bacteria. Scientists believe there may be many causes of ME/CFS, including various viral infections. Additionally, long COVID and other post-acute infection syndromes can evolve into ME/CFS. The exact percentages of people who suffer long-term consequences from different infections are not known, but it happens after different viral infections, and also with bacterial infections like Lyme disease.

There is an unmet need to address post-acute infection syndromes and to identify effective modulators of post-acute infection syndromes and their symptoms.

The inventions described in this patent relate to compounds and compositions for treating and/or preventing post-acute infection syndromes, including long-term sequalae of coronavirus infection and signs and symptoms of long-haul effects resulting from other infections, including but not limited to infections from coronaviruses. Coronaviruses are named for the crown-like spikes on their surface. There are four main sub-groupings of coronaviruses, known as alpha, beta, gamma, and delta. Human coronaviruses were first identified in the mid-1960s. The seven coronaviruses that can infect people are currently known to include: 229E (alpha coronavirus); NL63 (alpha coronavirus); OC43 (beta coronavirus); and HKU1 (beta coronavirus). Other human coronaviruses include: MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease, or COVID-19).

The present specification relates in part to compounds, compositions and methods having effects of treating one or more signs and/or symptoms of a post-acute infection syndrome. In some embodiments, the post-acute infection syndrome is long COVID. In some embodiments, the post-acute infection syndrome is long flu. In some embodiments, the post-acute infection syndrome is long cold. In some embodiments, the post-acute infection syndrome is post-treatment Lyme disease syndrome. In some embodiments, the post-acute infection syndrome is ME/CFS or another post-acute infection syndrome as described her or otherwise known in the art.

In some embodiments, the compounds, compositions and methods comprise at least one connexin hemichannel modulator. In some embodiments, the at least one connexin hemichannel modulator is a connexin 43 connexin hemichannel modulator.

In some embodiments, the compounds, compositions and methods comprise at least one inflammasome modulator. In some embodiments, the at least one inflammasome modulator is an NLRP3 inflammasome modulator.

The present specification relates in part to compounds, compositions and methods useful for treating one or more signs and/or symptoms of a post-acute infection syndrome. The present specification relates in part to compounds, compositions and methods useful for protecting against, alleviating, reducing or reversing, in whole or in part, one or more signs and/or symptoms of a post-acute infection syndrome. In some embodiments, the one or more signs and/or symptoms comprises fatigue. In some embodiments, the one or more signs and/or symptoms comprises brain fog. In some embodiments, the one or more signs and/or symptoms comprises another sign or symptom of a post-acute infection syndrome as described here and are known in the art, e.g. neuropathy, paresthesia, polyarthralgia, etc.

The present specification relates in part to compounds, to compositions containing such compounds, and methods of using the compounds and compositions for treating, preventing, alleviating and ameliorating a post-treatment disorder that is caused, in whole or in part by an infection, and symptoms thereof.

The present disclosure also provides, among other things, methods of screening for or quantifying the activity of connexin hemichannel modulating agents (e.g., connexin 43 hemichannel modulating agents) that can be used to treat a post-acute infection syndrome. In some embodiments, the inventions include methods of screening for or quantifying the activity of connexin hemichannel modulating agents (e.g., connexin 43 hemichannel modulating agents) that can be used to protect against one or more signs and/or symptoms of a post-acute infection syndrome.

The present disclosure also provides, among other things, methods of screening for or quantifying the activity of inflammasome modulating agents (e.g., NLRP3 inflammasome modulating agents) that can be used to treat a post-acute infection syndrome. In some embodiments, the inventions include methods of screening for or quantifying the activity of inflammasome modulating agents (e.g., NLRP3 inflammasome modulating agents) that can be used to protect against one or more signs and/or symptoms of a post-acute infection syndrome.

The present disclosure also provides methods of determining the efficacy of a connexin hemichannel modulating agent (e.g., a connexin 43 hemichannel modulating agent) to treat one or more signs or symptoms of a post-acute infection syndrome. The present disclosure also provides methods of providing a therapeutic (or higher) loading dose of a connexin hemichannel modulating agent (e.g., a connexin 43 hemichannel modulating agent) followed by lower maintenance dose to treat or prevent one or more signs and/or symptoms of a post-acute infection syndrome.

The present disclosure also provides methods of determining the efficacy of an inflammasome modulating agents (e.g., an NLRP3 inflammasome modulating agent) to treat one or more signs and/or symptoms of a post-acute infection syndrome. The present disclosure also provides methods of providing a therapeutic (or higher) loading dose of an inflammasome modulating agents (e.g., an NLRP3 inflammasome modulating agent) followed by lower maintenance dose to treat or prevent one or more signs and/or symptoms of a post-acute infection syndrome.

According to one aspect to the present invention, the compounds and compositions described herein decrease connexin hemichannel opening (e.g., connexin 43 hemichannel opening) to treat one or more signs or symptoms of a post-acute infection syndrome.

According to another aspect to the present invention, the compounds and compositions described herein decrease inflammasome activity (e.g., NLRP3 inflammasome activity) to treat one or more signs and/or symptoms of a post-acute infection syndrome.

In another aspect this invention relates to pharmaceutical compositions, articles of manufacture, kits and methods for treating a subject having (or at risk for having) a post-acute infection syndrome by administering a therapeutically effective amount of at least one connexin modulator (e.g., at least one connexin 43 modulator) to a subject or patient. In some embodiments, the agent is administered using a dose and/or dosing regimen described herein. In some embodiments, the agent is administered using another dose and/or dosing regimen. In some embodiments, one or more signs and/or symptoms of a post-acute infection syndrome is treated.

In another aspect this invention relates to pharmaceutical compositions, articles of manufacture, kits and methods for treating a subject having (or at risk for having) a post-acute infection syndrome by administering a therapeutically effective amount of at least one inflammasome modulating agent (e.g., at least one NLRP3 inflammasome modulating agent) to a subject or patient. In some embodiments, the agent is administered using a dose and/or dosing regimen described herein. In some embodiments, the agent is administered using another dose and/or dosing regimen. In some embodiments, one or more signs and/or symptoms of a post-acute infection syndrome is treated.

In one embodiment, a composition or compositions comprising a connexin modulator (e.g., a connexin 43 modulator) or an inflammasome modulator (e.g., an NLRP3 inflammasome inhibitor) or both is/are applied topically, systemically, etc., to a subject.

In some embodiments, the connexin modulator is a modulator of Cx26, Cx30, Cx31.1, Cx36, Cx37, Cx40, Cx43, Cx45, Cx50, Cx57 or any other connexin in the eye or blood vessels.

In some embodiments, the connexin modulator is a modulator of Cx26, Cx30, Cx30.3, Cx31, Cx31.1, Cx32, Cx43, Cx45, Cx50, and Cx58, or any other connexin in the epithelium. In some embodiments, the connexin modulator is a Cx43 connexin modulator, for example, a Cx43 expression modulator (e.g., an anti-Cx43 antisense), a Cx43 gap junction modulator or a Cx43 hemichannel modulator (e.g. an anti-connexin 43 peptidomimetic or a small molecule). In some embodiments, the modulator is a modified or unmodified antisense polynucleotide or peptidomimetic, e.g. a modified or unmodified Cx43 antisense polynucleotide or Cx43 peptidomimetic, or other corneal epithelial, endothelial or vascular connexin. In some embodiments, the modulator can include or exclude any of the foregoing connexins.

In some embodiments, the connexin hemichannel modulator administered daily, weekly, or monthly to a subject. In some embodiments, a therapeutically effective amount of tonabersat is administered.

In some embodiments, the inflammasome modulator (e.g., NLRP3 inflammasome inhibitor) is administered daily, weekly, or monthly to a subject. In some embodiments, a therapeutically effective amount of tonabersat is administered.

In some embodiments, the amount of a connexin modulator administered to a subject with one or more signs and/or symptoms of a post-acute infection disorder is one or more of the particular dose amounts described herein, including, for example, milligram per milliliter (mg/mL) connexin modulator dosing (e.g., a composition comprising or consisting essentially of about or at least about 1-100 mg/mL or more of a connexin hemichannel modulator), micromolar (μM) connexin modulator dosing (e.g., a composition comprising or consisting essentially of a concentration of about or at least about 100-500 μM or more of a connexin modulator), milligram (mg) connexin modulator dosing (e.g., a composition comprising or consisting essentially of about or at least about 40 to 160 mg or more of a connexin modulator), and so on, etc.

In some embodiments, the connexin hemichannel modulator comprises an orally available small molecule connexin hemichannel modulator (e.g., tonabersat, carabersat, or another small molecule connexin hemichannel modulator according to Formula I). See the Examples herein describing the use of an exemplary connexin hemichannel modulator (tonabersat). In some embodiments, the connexin hemichannel modulator is a prodrug of tonabersat (e.g., a tonabersat prodrug according to Formula II).

In some embodiments, the connexin modulator comprises a connexin peptidomimetic (e.g., a connexin 43 peptidomimetic). In some embodiments, the connexin peptidomimetic binds to an intracellular portion of a connexin (e.g. XG19). In some embodiments, the connexin peptidomimetic binds to an extracellular portion of a connexin (e.g. Peptide 5). Various connexin peptidomimetics, including connexin43 peptidomimetics, and methods of making them are known in the art.

In some embodiments, the connexin modulator comprises an antisense molecule (e.g., a connexin43 antisense oligonucleotide). In some embodiments, the connexin 43 antisense polynucleotide comprising, consisting essentially of, or consisting of a sequence according to SEQ ID NOS:1-3, SEQ ID NOS:4-16 and/or modified versions thereof. In some embodiments, the antisense oligonucleotide comprises, consists essentially of, or consists of 5′-GTA ATT GCG GCA AGA AGA ATT GTT TCT GTC-3′ (SEQ ID NO:1). In some embodiments, the antisense oligonucleotide may be chemically modified or may be an unmodified oligonucleotide, e.g. a modified or an unmodified DNA oligonucleotide. In some embodiments, oligonucleotides are modified by changing one or more of the phosphodiester linkages, the ribose backbone and/or the nucleobase. Other modifications are described herein. Other modifications are known in the art and described in various publications, including, for example, in Shen, X. and Corey, DR, Chemistry, mechanism and clinical status of antisense oligonucleotides and duplex RNAs, Nucleic Acids Res. 2018 Feb. 28; 46(4): 1584-1600. Methods of their manufacture are also known in the art. See, e.g., Abramova T, Frontiers and Approaches to Chemical Synthesis of Oligodeoxyribonucleotides, Molecules 2013 January; 18(1): 1063-1075; Hao M, et al., Current and Emerging Methods for the Synthesis of Single-Stranded DNA Genes (Basel) 2020 Jan. 21; 11(2):116. See also Anwar, S., et al. Enhancing the Effectiveness of Oligonucleotide Therapeutics Using Cell-Penetrating Peptide Conjugation, Chemical Modification, and Carrier-Based Delivery Strate and Carrier-Based Delivery Strategies. Pharmaceutics 2023, 15, 1130.

In some embodiments, a subject or patient is treated with a composition comprising or consisting essentially of 1-50 mg/mL SEQ ID NO:1. In some embodiments, the non-healing surface defect or disorder in a subject is treated with a composition comprising or consisting essentially of about 1-100 mg of SEQ ID NO:1 in the regimens described herein. In some embodiments, administration is to a cell, to a tissue or to an organ. In some embodiments, administration is to the brain. In some embodiments, administration is to the eye. In some embodiments, administration is to the skin. In some embodiments, the connexin modulator administered to a subject or patient (including in one or more amounts described herein) is a small molecule connexin hemichannel modulator (e.g., tonabersat, carabersat, or another small molecule connexin hemichannel modulator according to Formula I). In some embodiments, the connexin hemichannel modulator is a prodrug of tonabersat (e.g., a tonabersat prodrug according to Formula II). In some embodiments, the connexin modulator administered to a subject or patient (including in one or more amounts described herein) is a connexin antisense molecule. In some embodiments, the connexin modulator administered to a subject or patient (including in one or more amounts described herein) is a connexin peptidomimetic (e.g. XG19, Peptide 5, etc.).

In some embodiments, the therapeutically effective amount of the modulator, e.g. connexin modulator, for example a connexin 43 modulator, connexin 43 gap junction modulator and/or connexin 43 hemichannel modulator, which is effective in methods of the invention, comprises a composition that is about, or at least about, 10 micromolar (10 μM), 15 micromolar (15 μM), or 20 micromolar (20 μM), of a connexin modulator (e.g. SEQ ID NO:1), or any amount within or between any two of these recited dosages. Other effective doses that are effective in methods of the invention for the treatment of ocular surface defects or disorders (e.g. ocular PEDs and PCEDs) include about, or at least about, a composition comprising 30 micromolar (30 μM) or 40 micromolar (40 μM) of a connexin modulator, for example, a connexin 43 modulator, a connexin 43 gap junction modulator and/or a connexin 43 hemichannel modulator (e.g. SEQ ID NO:1). In some embodiments, the therapeutically effective amount of the connexin modulator administered as described is a connexin antisense molecule other than (or in addition to) SEQ ID NO:1. In some embodiments, the therapeutically effective amount of the connexin modulator administered as described is a connexin peptidomimetic (e.g. XG19). In some embodiments, the therapeutically effective amount of the connexin modulator as described is a connexin hemichannel blocker (e.g. tonabersat).

In some embodiments of these doses and dosing regimens and methods, the connexin modulator dose administered comprises another connexin antisense or modulator, e.g., another Cx43 antisense or Cx43 modulator other than or in addition to SEQ ID NO:1, or a modulator of another ocular or corneal epithelial connexin other than or in addition to Cx43.

In some embodiments, the modulator is a small molecule connexin hemichannel antagonist. In some embodiments, the modulator is a small molecule connexin hemichannel antagonist and comprises a compound according to Formula I, which includes tonabersat. Tonabersat, a benzoylamino benzopyran, is a modulator of connexin hemichannel activity, including connexin 43 hemichannels. Tonabersat can block or inhibit the opening of or release of molecules into the extracellular space from hemichannels comprising connexin 43.

In some embodiments, the modulator is a connexin peptidomimetic. Connexin modulators including Peptide5 inhibit Cx43 hemichannel activity and/or ATP release during and following injury. Other connexin modulator peptidomimetics include Gap19 and XG19, and analogues thereof.

The instant inventions provide, inter alia, (1) methods for treating, preventing or ameliorating one or more signs or symptoms of a post-acute infection disorder in a subject or a patient by administration of a therapeutically effective amount of one or more of a connexin hemichannel modulator (e.g., a connexin 43 expression modulator, a connexin 43 gap junction modulator, a connexin 43 hemichannel modulator, etc.) and an inflammasome modulator (e.g. an NLRP3 inflammasome modulator) in a dose regimen described herein; (2) methods for treating, preventing or ameliorating one or more signs or symptoms of a post-acute infection disorder in a subject or a patient (e.g. a long COVID patient, for example, or another long hauler) by administration of a connexin modulator and/or an inflammasome modulator at doses described herein (e.g. in any of the dosing regimens described herein; and (3) the use of such modulators, including, for example, anti-connexin and/or anti-inflammasome antisense modulators, peptidomimetic modulators and small molecule modulators, in the manufacture of a medicament (e.g. a pharmaceutical composition), and kits containing said medicaments that include or are associated with instructions for use in a method of the invention.

In some embodiments, instant inventions provide, inter alia, (1) methods for treating one or more signs and/or symptoms of a post-acute infection syndrome (e.g., long COVID, long flu, post-treatment Lyme disease syndrome, etc.) in a subject or a patient by administration of a therapeutically effective amount of at least one connexin modulator (e.g., a connexin expression modulator, a connexin gap junction modulator and/or a connexin hemichannel modulator) and/or at least one inflammasome modulator in a dose regimen described herein to treat the disease, disorder or condition; (2) methods for treating one or more signs and/or symptoms of a post-acute infection syndrome (e.g., long COVID, long flu, post-treatment Lyme disease syndrome, etc.) in a subject or a patient by administration of the connexin modulator and/or inflammasome doses described herein (e.g. in any of the dosing regimens described herein); and (3) the use of such connexin modulators and/or inflammasome modulators, including, for example, small molecule modulators, peptidomimetic modulators and antisense modulators in the manufacture of a medicament (e.g. a pharmaceutical composition), and kits containing said medicaments that include or are associated with instructions for use in a method of the invention for treating one or more signs and/or symptoms of a post-acute infection syndrome (e.g., long COVID, long flu, post-treatment Lyme disease syndrome, etc.).

In some embodiments, the symptom to be treated is a long-hauler symptom resulting following treatment of an infection.

In some embodiments, one or more of the connexin modulators described or referenced herein are formulated with one more other therapeutically or prophylactically active agents for use in the methods and regimens described herein. In some embodiments, one or more of the inflammasome modulators described or referenced herein are formulated with one more other therapeutically or prophylactically active agents for use in the methods and regimens described herein.

### Definitions

Important inventions protected by issued patents are often copied by infringers. Those who defend accused patent infringers do so before judges and jurors who generally have little or no scientific background or technical knowledge, and will often try to avoid infringement by arguing that claims in a patent don't mean what they say. They will argue about common words and accepted scientific or technical terms, no matter how well known or easily understood. They also hire “experts” who will testify that they do not understand well-known words or terms and assert that the patent doesn't sufficiently “describe” an invention, or the meaning of the words used in a claim. No words or arguments are off-limits for accused patent infringers. The below definitions are provided to hold off those who would take one or more of the inventions described and claimed herein, and then argue that the patent covering the invention they took should be held invalid because they allegedly don't understand one or more the words it uses, or by seeking to alter the meaning of one or more words to try and avoid liability for infringement.

As used herein, the singular forms “a,” “an” and “the” include plural referents unless the content clearly dictates otherwise. Thus, for example, reference to a “connexin hemichannel blocker” or an “inflammasome inhibitor” optionally includes a combination of two or more such molecules, and the like.

As used herein, the terms “about” and “approximately,” when used to modify a numeric value (e.g., “about 80 mg”) or one or more amounts specified in a range of numeric values (e.g., “about 80 mg to about 400 mg”), indicate the numeric value and functionally equivalent values, as well as reasonable deviations from the value known to or understood by those in the art, including persons of ordinary skill in the art or those skilled in the art. For example, values within ±10% or ±5% are within the intended meaning of a recited value. As such, “about 80 mg” is understood to encompass from “76 mg to 84 mg” or from “72 mg to 88 mg” as if written out each time. Thus, as used herein, the terms “about” and “approximately” intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise. In other words, when ranges are expressed herein as from “about” one particular value, and/or to “about” another particular value, the range also includes from the one particular value and/or to the other particular value, including the variations noted above or understood in the art. For example, if the value “10” is disclosed, then “about 10” is also disclosed. It is also understood that each unit between two particular units are also disclosed. For example, if “10 to 15” (or “10-15”) is disclosed, then 11, 12, 13, and 14 are also specifically disclosed (even it not spelled out in writing), in addition to 10 and 15, as well as “about 11,” “about 12,” “about 13,” and “about 14.”

As used herein, the terms “administering” and “administration” refer to any method of providing a compound or composition to a subject. In some embodiments, the compound or composition is a pharmaceutical preparation. Such methods are well known to those skilled in the art and include, but are not limited to, oral administration, transdermal administration, administration by inhalation, nasal administration, topical administration, intravaginal administration, ophthalmic administration, intraaural administration, intradural administration, intracerebral administration, rectal administration, sublingual administration, buccal administration, and parenteral administration, including injectable such as intravenous administration, intra-arterial administration, intramuscular administration, and subcutaneous administration. Administration can be one-time, intermittent, or continuous. In some embodiments, a compound or composition of the invention is administered therapeutically, e.g., to treat an existing, diagnosed or suspected disease, disorder or condition. In some embodiments, a compound or composition of the invention is administered prophylactically, e.g., administered to prevent or inhibit progression of a disease, disorder or condition, or prevent the spread of a disease, disorder or condition.

As used herein, an “antisense compound,” also referred herein to as “antisense polynucleotides” or “antisense oligonucleotides” (also known as ASOs or AONs), means a compound comprising or consisting of an oligonucleotide, at least a portion of which is complementary to a target nucleic acid to which it is capable of hybridizing, thereby resulting in at least one antisense activity. As used herein, “antisense activity” means any detectable and/or measurable change attributable to hybridization of an antisense compound to its target nucleic acid. In certain embodiments, antisense activity comprises modulation of the amount or activity of a target nucleic acid transcript (e.g., mRNA). In certain embodiments, antisense activity comprises modulation of splicing of a precursor mRNA. Antisense compounds, and their design, manufacture and use are well known in the art. In some embodiments, they comprise single stranded pieces of DNA that match the complementary sequence of a specific mRNA (e.g., a connexin mRNA). In some embodiments, the antisense activity of an antisense compounds prevents or alters the production of a protein (e.g., pannexin 1). In some embodiments, the antisense compound is a connexin 43-directed antisense oligonucleotide. In some embodiments, the antisense compound is an NLRP3-directed antisense oligonucleotide. NLRP3-specific ASOs are known in the art. In some embodiments, the NLRP3-directed antisense oligonucleotide is directed to NLRP3 regions predicted to be in an open, as opposed to folded, conformation based, for example, on their minimum free energy prediction. See, e.g. Braatz, C, et al. (2023). NLRP3-directed antisense oligonucleotides reduce microglial immunoactivities in vitro. Journal of Neurochemistry, 2023 00:1-15. https://doi.org/10.1111/jnc.15778. Kaufmann B. et al. Antisense Oligonucleotide Therapy Decreases IL-1β Expression and Prolongs Survival in Mutant Nlrp3 Mice. J Immunol. 2023 211(2):287-294. In some embodiments, the antisense compound is a pannexin 1-directed antisense oligonucleotide. In some embodiments, antisense polynucleotides or oligonucleotides comprise unmodified nucleotides. In some embodiments, antisense polynucleotides or oligonucleotides comprise one or more modified nucleotides. Many different chemical modifications are currently available. See, e.g., Crooke S T, et al. Antisense technology: an overview and prospectus. Nat Rev Drug Discov. 2021 20(6):427-453; Crooke S T, et al. Antisense technology: A review. J Biol Chem. 2021 296:100416; Kuijper E C, et al. Opportunities and challenges for antisense oligonucleotide therapies. J Inherit Metab Dis. 2021 44(1):72-87; Dhuri K, et al. Antisense Oligonucleotides: An Emerging Area in Drug Discovery and Development. J Clin Med. 2020 9(6):2004. Many articles, book chapters and books have been written that are devoted to all aspects of the design, manufacture and use of antisense technologies, as well as issued patents (see, e.g., U.S. Pat. No. 9,382,540, issued Jul. 5, 2016, for “Compositions and methods for modulating angiopoietin-like 3 expression), the detail of which are all well-known to those having even ordinary skill in the art and need not be detailed here.

As used herein, the term “connexin hemichannel modulator” (which may also be referred to herein as a connexin hemichannel, connexin, or hemichannel “inhibitor,” “blocker” or “antagonist”) is a compound that prevents, inhibits, and/or reduces the formation, function or activity of a connexin hemichannel. Connexin hemichannel modulators include chemical-based connexin hemichannel modulators (e.g. tonabersat, carabersat or other compounds within Formula I, or pro-drugs thereof, e.g. compounds of Formula II, which are prodrugs of tonabersat), peptide-based connexin hemichannel modulators (e.g. XG19, Peptide 5, aCT1 peptides, etc.), DNA- and RNA-based connexin hemichannel modulators (e.g. SEQ ID NO:1, etc.), antibody- and antibody fragment-based connexin hemichannel modulators (e.g. monoclonal antibodies, ScFvs, etc.). See, e.g., Willebrords J, et al. Inhibitors of connexin and pannexin channels as potential therapeutics. Pharmacol Ther. 2017 180:144-160. In some embodiments, the connexin hemichannel modulators are connexin 43 hemichannel modulators. Connexin hemichannels are normally closed to maintain cellular homeostasis, but can be activated in pathophysiological processes to serve as toxic membrane pores. For this reason, connexin hemichannels are sometimes considered as “pathological pores” (see Decrock E, et al., Connexin-related signaling in cell death: to live or let die? Cell Death Differ. 16(4):524-36 (2009), and the opening of connexin hemichannels facilitates the release of damage-associated molecular patterns (DAMPs), a class of endogenous molecules related to the pathogenesis of inflammatory diseases. In some embodiments, the connexin hemichannel modulator will block, inhibit or reduce the pathological opening of a connexin hemichannel (e.g., pathological opening a connexin 43 hemichannel). In some embodiments, pathological opening of a connexin hemichannel (e.g., pathological opening a connexin 43 hemichannel) results in the release of ATP into the extracellular space, which is blocked, inhibited or reduced by a connexin hemichannel modulator (e.g., a connexin 43 hemichannel modulator).

Modulation of a function and/or activity of a connexin hemichannel includes, for example, the prevention, inhibition and/or reduction, in whole or in part, in the expression of a connexin protein, its trafficking and/or assembly as a hemichannel (e.g., a connexin 43 hemichannel). In some embodiments, modulation of a function and/or activity of a connexin hemichannel comprises modulating, blocking, reducing or inhibiting of the flow of molecules through a hemichannel (e.g., a connexin 43 hemichannel), such as ATP or DAMPs, for example. It includes, for example, the flow of molecules from the extracellular space or environment through a hemichannel (e.g., a connexin 43 hemichannel) into a cell, and/or the flow of molecules (e.g. adenosine triphosphate (ATP) or a DAMP) through a connexin hemichannel from the intracellular space or environment of a cell into the extracellular space or environment. Thus, by way of example, modulation may occur by one or more of: reducing, preventing, blocking, inhibiting or decreasing hemichannel formation; inducing or promoting closure of a hemichannel; reducing, preventing, blocking, inhibiting or decreasing hemichannel opening; reducing, preventing, blocking, inhibiting or decreasing hemichannel permeability; inducing coupling between hemichannels; triggering, inducing or promoting cellular internalization of a connexin hemichannel and/or gap junction. Words such as “blocking”, “inhibiting”, “preventing”, “decreasing” and “antagonizing”, and the like, may not be taken to imply complete reducing, blocking, inhibition, prevention, or antagonism. Similarly, “inducing” or “promoting” is not be understood to require complete induction or promotion of formation of a gap junction through hemichannel docking, inducing or promoting the complete closure of a hemichannel; completely preventing, blocking, inhibiting or decreasing hemichannel opening; completely preventing, blocking, inhibiting or decreasing hemichannel permeability; completely inducing coupling between hemichannels; or triggering, inducing or promoting the complete loss of coupling or the complete internalization of a connexin hemichannel and/or gap junction (or group of hemichannels and/or gap junctions) and should be taken to include the partial induction or promotion of any of them.

Modulation of a connexin hemichannel includes prevention, inhibition and/or reduction, in whole or in part, of pathological opening of a connexin hemichannel (e.g., pathological opening a connexin 43 hemichannel). In some embodiments, pathological opening of a connexin hemichannel (e.g., pathological opening a connexin 43 hemichannel) results in the release of ATP into the extracellular space.

Modulation of one or more functions or activities of a connexin hemichannel may occur by any means. Additionally, preventing, blocking, inhibiting and/or reducing a function or activity of a connexin hemichannel (e.g., a connexin 43 hemichannel) may be direct or indirect. Preventing, blocking, inhibiting and/or reducing connexin hemichannel function or activity includes, for example, directly blocking a connexin hemichannel, inducing a conformational change, and modifying a connexin phosphorylation state or its open-close probability. Open probability is a measure of the percentage of time a channel remains open versus being closed (reviewed in Goldberg G S, et al., Selective permeability of gap junction channels Biochimica et Biophysica Acta 2004 1662:96-101).

Modulation of a connexin hemichannel also includes suppressing the permeability of a connexin hemichannel (for example, lessening the flow of molecules through a connexin hemichannel, e.g., a connexin 43 hemichannel). In some embodiments, the molecule is adenosine triphosphate (ATP). Thus, in some embodiments, a “hemichannel blocker” is a compound that interferes with the passage of molecules through a connexin hemichannel. A hemichannel blocker can block or decrease hemichannel opening, block, decrease or suppress hemichannel opening, block or reduce the release of molecules through a hemichannel to an extracellular space, and/or block or reduce the entry of molecules through a hemichannel into an intracellular space. Hemichannel blockers include compounds that fully or partially block hemichannel leak or the passage of molecules through a hemichannel (e.g. to or from an extracellular space). Hemichannel blockers also include compounds that decrease the open probability of a hemichannel. Examples of hemichannel blockers include peptides, peptidomimetics, small molecules, antibodies and antibody fragments. Hemichannel blockers include hemichannel modulators and may interfere directly or indirectly with the passage of molecules through a connexin hemichannel, or with the permeability of a connexin hemichannel (e.g., a connexin 43 hemichannel).

The modulator may be of any chemical nature. As noted, by way of example, the connexin hemichannel modulator may be a nucleic acid (including antisense molecules, RNAi molecules, morpholinos, and other nucleic acids as described herein or known in the art), a peptide or peptidomimetic, or a small molecule or other chemical. Connexin modulators include connexin expression modulators, connexin gap junction modulators and connexin hemichannel modulators, including connexin gap junction modulators that can modulate a connexin hemichannel. In some embodiments, connexin expression modulators, connexin gap junction modulators and/or connexin hemichannel modulators are compounds can modulate—i.e., inhibit, block, prevent, reduce or antagonize, in whole or in part, the function, activity, expression, trafficking and/or assembly of—a connexin 43 hemichannel. However, as stated, the words “inhibit” or “block” or “antagonize” or “modulate” or “prevent” shall not be taken to imply that the function, activity, expression, trafficking and/or assembly of a connexin, a connexin hemichannel (e.g. a connexin 43 hemichannel) or gap junction (e.g. a connexin 43 gap junction) is completely inhibited or blocked or wholly antagonized or modulated or prevented, but should be understood to include any reduction or inhibition in the function, activity, expression, trafficking and/or assembly of a connexin (including its transcription, translation and/or expression), a connexin hemichannel (including its permeability or its opening, or release of ATP, to the extracellular environment) or gap junction (including its opening to an adjacent cell, or creation from hemichannels in adjacent cells).

Hemichannels and gap junction channels may be present in cells of any type. The modulation of connexins, hemichannels and gap junctions may be in cells of any type. Accordingly, reference to a “connexin,” “hemichannel” or “gap junction” should be taken to include reference to a connexin, a hemichannel or gap junction present in any cell type, unless the context requires otherwise. In some embodiments, the connexin, hemichannel or gap junction is present in an epithelial cell. In some embodiments, the connexin, hemichannel or gap junction is present in an endothelial cell. In some embodiments, the connexin, hemichannel or gap junction is a vascular connexin, hemichannel or gap junction. In some embodiments, the connexin, hemichannel or gap junction is a connexin, hemichannel or gap junction found in vascular endothelial cells and/or vascular smooth muscle cells. In some embodiments, the connexin, hemichannel or gap junction is present in a brain cell, a retinal cell and/or in other cells noted herein or that are targeted in carrying out a method of the invention.

In some embodiments, the connexin modulator may be a modulator of a connexin hemichannel present in blood vessels, for example, a connexin 43 hemichannel modulator and/or connexin 37 hemichannel modulator, a connexin 40 hemichannel modulator, a connexin 45 hemichannel modulator or another blood vessel connexin hemichannel. In some embodiments, the connexin modulator may be a modulator of a connexin hemichannel present in ocular cells, e.g., in cells of the retina. In some embodiments, the connexin modulator may be a modulator of a connexin hemichannel present in brain cells, e.g., in cells of the amygdala, hippocampus, hypothalamus and thalamus. In some embodiments, the connexin modulator may be a modulator of a connexin hemichannel present in glial and/or microglial cells. In some embodiments, the connexin modulator is a modulator of a Cx26, Cx30, Cx30.3, Cx31, Cx31.1, Cx32, Cx43, Cx45, Cx50, and/or Cx58 hemichannel, or any other connexin hemichannel in the corneal epithelium. In other embodiments, the connexin modulator is a modulator of any other connexin hemichannel in the eye or in blood vessels, including Cx36 and Cx57 hemichannels in addition to Cx37, Cx40, Cx43 and Cx45 hemichannels.

In some embodiments, the connexin hemichannel modulator is a connexin 43 hemichannel modulator. In some embodiments, the connexin hemichannel modulator is a modulator of another connexin hemichannel. In some embodiments, the connexin hemichannel modulator is a modulator of one or more of a connexin 26 hemichannel, a connexin 30 hemichannel, a connexin 30.3 hemichannel, a connexin 31 hemichannel, a connexin 31.1 hemichannel, a connexin 32 hemichannel, a connexin 45 hemichannel, a connexin 50 hemichannel, and a connexin 58 hemichannel. In some embodiments, the connexin modulator may be a modulator of a connexin present in blood vessels or endothelial cells, for example, a connexin 37 hemichannel modulator, or a connexin 49 hemichannel modulator, for example. Thus, as used herein, the term “connexin hemichannel modulator” refers generally to connexin hemichannel modulators, but also specifically to connexin 43 hemichannel modulators and modulators of other connexin hemichannels referenced herein, or any other desired target connexin hemichannel. In some embodiments, the connexin hemichannel modulator is a connexin 43 hemichannel modulator, e.g., a modulator of a connexin 43 expression, a connexin 43 hemichannel modulator that inhibits or blocks hemichannel opening including small molecule and peptidomimetic modulators of connexin 43 hemichannel. In some embodiments, the connexin hemichannel modulating agent can include or exclude any of the connexins or connexin hemichannels referenced herein.

The phrase “consisting essentially of” refers to specified materials and those additional materials that do not materially affect the basic and novel characteristics of a composition or medicament (and/or steps, in the case of a method). Basic and novel characteristics of the inventions are described throughout the specification in the context of the discussion. They include the ability of connexin hemichannel modulators and connexin hemichannel modulator compositions and methods of use thereof to treat or prevent one or more signs and/or symptoms (e.g., fatigue, brain fog, etc.) of a post-acute infection syndrome (e.g., long COVID, long flu, post-treatment Lyme disease syndrome, etc., and other post-acute infection syndromes). They also include the ability of connexin hemichannel modulators, connexin hemichannel modulator compositions and methods of using connexin hemichannel modulators and connexin hemichannel modulator compositions to block or modulate connexins, connexin gap junctions and/or connexin hemichannels (e.g., connexin 43 connexins, gap junctions and/or hemichannels) and/or inflammasomes (e.g., NLRP3 inflammasomes). They also include the ability of connexin modulators, connexin modulator compositions and methods of using connexin modulators and connexin modulator compositions to block or modulate one or more connexins (e.g., connexin 43), the expression of one or more connexins (e.g., connexin 43 expression), the activity of one or more connexins (e.g., connexin 43 activity), the formation of connexin gap junctions (e.g., connexin 43 gap junction) and/or the formation of connexin hemichannels (e.g., connexin 43 hemichannels). Basic and novel characteristics of connexin hemichannel modulators, connexin hemichannel modulator compositions and methods of using connexin hemichannel modulators and connexin hemichannel modulator compositions also include the ability of connexin hemichannel modulators and connexin hemichannel modulator compositions to block or modulate the release of ATP and/or DAMPs from connexin hemichannels. Basic and novel characteristics of inflammasome modulator inventions include the ability of inflammasome modulators, inflammasome modulator compositions, and methods of using inflammasome modulators and inflammasome modulator compositions to treat or prevent one or more signs and/or symptoms (e.g., fatigue, brain fog, etc.) of a post-acute infection syndrome (e.g., long COVID, long flu, post-treatment Lyme disease syndrome, etc., and other post-acute infection syndromes). They also include the ability of inflammasome modulators, inflammasome modulator compositions, and methods of use thereof to block, inhibit or otherwise modulate the activity of one or more inflammasomes (e.g., NLRP3 inflammasome activity). They also include the ability of inflammasome modulators, inflammasome modulator compositions, and methods of use thereof to block, inhibit or otherwise modulate the activation of one or more inflammasomes (e.g., activation of the NLRP3 inflammasome). They also include the ability of inflammasome modulators, inflammasome modulator compositions, and methods of use thereof to block, inhibit or otherwise modulate activation of an inflammatory cascade by one or more inflammasomes (e.g., activation of an inflammatory cascade by the NLRP3 inflammasome). Basic and novel characteristics of pannexin modulator inventions include the ability of pannexin modulators, pannexin modulator compositions, and methods of using pannexin modulators and pannexin modulator compositions (e.g., Panx1 modulators and Panx1 modulator compositions) to treat or prevent one or more signs and/or symptoms (e.g., fatigue, brain fog, etc.) of a post-acute infection syndrome (e.g., long COVID, long flu, post-treatment Lyme disease syndrome, etc., and other post-acute infection syndromes). They also include the ability of pannexin modulators, pannexin modulator compositions, and methods of use thereof to block or modulate pannexin (e.g., Panx 1) or release of ATP from pannexin channels (e.g., Panx 1 channels). Material changes in the basic and novel characteristics of inventions, including the compositions, medicaments and methods described herein, include changes or additions that result in unwanted activity, materially diminished activity or clinically materially undesirable activity. Material changes in the basic and novel characteristics of inventions, including the compositions, medicaments and methods described herein, include changes or additions that result in detrimental, disadvantageous or adverse activity. Material changes in the basic and novel characteristics of inventions, including the compositions, medicaments and methods described herein, include changes or additions resulting in detrimental, disadvantageous or adverse activity in treating or preventing a disease, disorder or condition described or referred to herein, for example, or an unwanted or undesirable diminution of the activity of a connexin hemichannel modulator (e.g. a connexin 43 hemichannel modulator), an inflammasome modulator (e.g. an NLRP3 inflammasome modulator) and/or a pannexin modulator (e.g. a pannexin 1 channel modulator). In some embodiments, a composition or medicament of the invention will comprise, consist essentially of, or consist of at least one connexin 43 hemichannel modulator, for example, a connexin 43 antisense molecule, a connexin 43 peptidomimetic hemichannel modulator or small molecule connexin 43 hemichannel blocker (e.g., tonabersat or another compound of Formula I) or a prodrug thereof (e.g., a tonabersat prodrug according to Formula II). In some embodiments, a composition or medicament of the invention will comprise, consist essentially of, or consist of at least one NLRP3 inflammasome modulator, for example, a direct NLRP3 inflammasome modulator and/or an indirect NLRP3 inflammasome modulator. In some embodiments, a composition or medicament of the invention will comprise, consist essentially of, or consist of at least one antisense, peptidomimetic or small molecule NLRP3 inflammasome modulator. In some embodiments, a composition or medicament of the invention will comprise, consist essentially of, or consist of at least one pannexin 1 channel modulator, for example, a pannexin 1 antisense molecule, a pannexin 1 peptidomimetic or small molecule pannexin 1 hemichannel blocker or prodrug thereof.

As used herein, “effective amount” or “therapeutically effective amount” refers to an amount effective, at dosages and for periods of time as described in the dose regimens herein, to achieve a desired result in treatment of a disease, disorder, defect or condition for which one or more compounds or compositions of the invention are administered to a subject or to a patient. For example, and not by way of limitation, a “therapeutically effective amount” can refer to an amount of a connexin hemichannel modulator compound or composition (e.g. a compound or composition comprising or consisting essentially of a connexin 43 hemichannel modulator), to an amount of an inflammasome modulator compound or composition (e.g. a compound or composition comprising or consisting essentially of an NLRP3 inflammasome inhibitor), or to an amount of a pannexin channel modulator (e.g. a compound or composition comprising or consisting essentially of a pannexin 1 channel modulator), including but not limited to those disclosed herein, that is able to treat a disease, defect, disorder or condition when administered in accordance with the invention.

In some embodiments, “effective amount” or “therapeutically effective amount” refers to an amount effective a compound or composition, at dosages and for periods of time as described in the dose regimens herein or used by those in accordance with methods of the invention, to achieve a desired result or results in the treatment of one or more signs and/or symptoms of a post-acute infection syndrome (e.g., long COVID, long flu, post-treatment Lyme disease syndrome, etc., and other post-acute infection syndromes). Symptoms include those described or referenced herein, for example, post-acute infection fatigue, brain fog, etc. Therapeutically effective amounts include an amount that is sufficient to achieve at least one desired therapeutic result, or to have at least one effect on at least one undesired symptom associated with a disease, disorder, defect or condition, including but not limited to those referenced herein. The specific therapeutically effective dose level for any particular patient may be varied or depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors used in the medical arts in determining dose levels. For example, those in the art may start doses of a compound or composition at levels lower than those required to achieve the desired therapeutic effect and to gradually increase the dosage until the desired effect is achieved. In some embodiments, the result of treatment with an “effective amount” provides a durable result following treatment. In some embodiments, treatment with an effective amount is discontinued following a desired result. In some embodiments, treatment with an effective amount is continued following a desired result. In some embodiments, treatment with an effective amount is paused and then continued following a desired result. The doses disclosed herein are therapeutically effective amounts; however, the methods are not limited to those doses or dose amounts and include the use of other therapeutically effective amounts.

In some embodiments, a compound, composition or preparation can be administered in a “prophylactically effective amount”; that is, an amount effective for prevention of a disease, disorder or condition, including one or more signs and/or symptoms of a post-acute infection syndrome described or referenced herein (e.g., long COVID, long flu, post-treatment Lyme disease syndrome, etc., and other post-acute infection syndromes). As used herein, “prophylactically effective amount” refers to an amount effective to achieve or assure a desired prophylactic result, including, for example, treating or preventing one or more signs and/or symptoms of a post-acute infection syndrome described or referenced, including, for example, fatigue, brain fog, etc. The compounds and compositions herein can be used to prevent, inhibit, or reduce the occurrence of, one or more signs or symptoms of a post-acute infection syndrome.

A “maintenance dose” or a “preventative dose” is a dose intended to be a therapeutically effective or a prophylactically effective dose. In some embodiments, the maintenance doses may be therapeutically effective doses and others may be sub-therapeutic doses. We have discovered dosing regimens and oral dosage formulations for the administration of maintenance doses of connexin hemichannel modulators and inflammasome modulators. For example, in some embodiments, the invention features a method of treating one or more signs and/or symptoms of a post-acute infection syndrome in a subject by (i) administering a loading-dose of a connexin hemichannel modulator and/or an inflammasome modulator to the subject; and (ii) administering one or more maintenance doses of the connexin hemichannel modulator and/or an inflammasome modulator to the subject, wherein each of the loading-dose and the maintenance doses are administered in an amount that together are sufficient to treat the one or more signs and/or symptoms of a post-acute infection syndrome. For example, the loading dose can be administered by injection (e.g., subcutaneously) or orally followed by maintenance dosing administered orally, intravenously, nasally, subcutaneously or transdermally. In sone embodiments, the loading-dose is administered orally. In some embodiments, the loading-dose is administered by injection or infusion. For example, the intravenously (e.g., as a bolus or infusion) administered loading-dose can be administered in an amount sufficient to produce a mean steady-state concentration of the connexin hemichannel modulator and/or an inflammasome modulator in the subject. In some embodiments, the introductory or loading dose is about 40 mg to about 100 mg (or other dose described herein) administered daily and the maintenance dose is about 100 mg to about 200 mg administered about once a week. In some embodiments, the introductory dose is about 80 mg to about 200 mg and the dose in the maintenance period is about 200 mg to about 300 mg administered once a week. In some embodiments, the dose for the induction phase, e.g., the introductory or loading dose, is a dose of 100 mg administered at least once per day and the dose for the maintenance phase is about 200 mg once a week.

As used herein, a “gap junction,” a “connexin gap junction” and a “connexin gap junction channel” refer to two hemichannels connected across an intercellular space between adjacent cells that allow certain molecules to flow between the cells. They refer to the intercellular channels or clusters of intercellular channels (gap junction plaques) that allow direct diffusion of ions and small molecules between adjacent cells. Gap junctions have been studies for decades and are well known in the art.

All connexins share the same topological structure, including four transmembrane domains, two extracellular loops (EL1 and EL2), a cytoplasmic loop (CL) and amino (NT) and carboxyl termini (CT) in the cytoplasm. The carboxyl termini exhibit the greatest heterogeneity in amino acid sequences, whereas the transmembrane and extracellular domains are most conserved among the connexin isoforms. In general, connexins are expressed in the endoplasmic reticulum, transported to the Golgi apparatus to form hexamers (connexons), trafficked to the plasma membrane as connexin hemichannels, which then flow laterally to fuse with gap junction plaque and assemble with connexin hemichannels from apposing cells, forming into gap junctions. Thus, connexin hemichannels exist on plasma membranes before forming gap junctions. The presence of connexin hemichannels, however, does not always lead to the formation of gap junctions. For example, hemichannels formed by connexin 43, the most widely expressed connexin, can remain non-junctional on cell membranes if adequate adhesion molecules are not expressed. Non-junctional connexin structures are also reported in vivo. In one study using atomic force microscopy, hemichannels were found to account for about 12-17% of the total number of plaques in gap junction preparations from rat hearts (Lal R, John S A, Laird D W, Arnsdorf, MF Heart gap junction preparations reveal hemiplaques by atomic force microscopy. Am. J. Physiol, 1995 268:C968-C977), suggesting that connexin hemichannels may possess substantial permeation capacity in some critical organs. Connexin hemichannels are normally closed to maintain cellular homeostasis, but can be activated in pathophysiological processes to serve as toxic membrane pores, and have been referred to by some as “pathological pores.” As used herein, the term “hemichannel” (or “connexin hemichannel”) is a structure comprised of connexin proteins, typically homo- or hetero-meric hexamers of connexin proteins that form the pore for a gap junction between the cytoplasm of two adjacent cells. The hemichannel is supplied by a cell on one side of the junction, and by a cell on the other side, with two hemichannels from opposing cells normally coming together to form the complete intercellular gap junction channel. However, as noted, in some cells, and in cells under some circumstances, the hemichannel itself is active as a conduit between the cytoplasm and the extracellular space allowing the transference of ions and small molecules (e.g. ATP). Like their gap junction counterparts, connexin hemichannels have been long-studied and are also well known in the art.

Increased connexin 43 hemichannel opening is associated with inflammasome pathway activation and inflammation. As used herein, the term “inflammasome modulator” (which may also be referred to as an inflammasome “inhibitor,” “blocker” or “antagonist”) is a compound that directly or indirectly prevents, inhibits, and/or reduces the formation, function or activity of an inflammasome (e.g., an NLRP3 inflammasome), including, for example, prevention, inhibition and/or reduction in the function and/or activity and/or the formation of an inflammasome (e.g., an NLRP3 inflammasome), including its assembly. In some embodiments, activation of an inflammatory cascade by the NLRP3 inflammasome is modulated (e.g. inhibited, blocked or downregulated) by a direct NLRP3 inflammasome modulator. In some embodiments, activation of an inflammatory cascade by the NLRP3 inflammasome is modulated (e.g. blocked or downregulated) by an indirect modulator of the NLRP3 inflammasome. In some embodiments, a direct or indirect inflammasome modulator may include or exclude any known direct or indirect inflammasome modulator. In some embodiments, activation and/or activity of the NLRP3 inflammasome is modulated (e.g. blocked, inhibited or downregulated) by a connexin hemichannel modulator. In some embodiments, an inflammasome modulator may include a connexin hemichannel modulator. In some embodiments, an inflammasome modulator may exclude a connexin hemichannel modulator. Both connexin hemichannel modulation (e.g. connexin 43 hemichannel modulation) and inflammasome modulation (e.g. NLRP3 inflammasome modulation) are useful in treating or preventing one or more of the diseases, defects, disorders or conditions described or referenced herein (e.g., long COVID, long flu, post-treatment Lyme disease syndrome, etc., and other post-acute infection syndromes). Both connexin hemichannel modulation (e.g. connexin 43 hemichannel modulation) and inflammasome modulation (e.g. NLRP3 inflammasome modulation) are useful in treating or preventing one or more signs and/or symptoms of a post-acute infection syndrome (e.g. fatigue, brain fog, etc.). Pannexin channel modulators and pannexin channel modulator compositions (e.g. Panx1 channel modulators and compositions) will also be useful in treating and/or preventing one or more signs and/or symptoms of a post-acute infection syndrome.

In some embodiments, a compound, composition or preparation can be administered in a “prophylactically effective amount”; that is, an amount effective for prevention of a disease, disorder or condition, including one or more signs and/or symptoms of a post-actue infection syndrome described or referenced herein (e.g., long COVID, long flu, post-treatment Lyme disease syndrome, etc.). As used herein, “prophylactically effective amount” refers to an amount effective to achieve or assure a desired prophylactic result, including, for example, treating or preventing one or more signs and/or symptoms of a post-acute infection syndrome described or referenced, including, for example, fatigue, brain fog, etc.

In some embodiments, a disease, defect, disorder or condition is treated with an inflammasome modulator (e.g. a modulator of the NLRP3 inflammasome) that may not be a connexin modulator (e.g. a connexin 43 hemichannel modulator). Inflammasome modulators include those described, for example, in Leung and Lowery, The patent landscape of inflammasome modulators. Nature Reviews Drug Discovery 19, 158 (2020). See also Chauhan, D., et al., Therapeutic modulation of inflammasome pathways. Immunol Rev. 297(1): 123-138 (September 2020). Inflammasome modulators include selective G-protein coupled receptor 40 (GPR40) agonists (e.g. fasiglifam, which inhibits inflammasome activation by blocking formation of apoptosis-associated speck-like protein (ASC) containing a caspase recruitment domain (CARD), an inflammasome component), and ethyl pyruvate, which significantly suppresses activation of the NLRP3 inflammasome. Other inflammasome modulators unrelated to connexins and connexin hemichannels are known in the art.

The term “in vivo” (Latin for “within the living”) refers to use in a whole, living organism as opposed to an ex vivo or in vitro controlled environment. Animal testing, clinical trials, and use in humans or other living subjects are forms of in vivo research. In vivo testing is often employed over in vitro to observe the overall effects of an experiment on a living subject. Ex vivo work or experimentation is done in live isolated cells, tissues or organs, rather than in a whole organism, for example, cultured cells derived from biopsies. Once cells are disrupted and individual parts are tested or analyzed, this is referred to as in vitro. Methods carried out in vivo include the use of animal models for research. In vivo methods also include the use of methods in humans and other subjects, including for therapy. The term “ex vivo” (Latin: “out of the living”) refers to use outside an organism, and is used to indicate methods, work, experimentation or measurements done in or on tissue in an artificial environment outside an organism. Ex vivo work is often done with the minimum alteration of natural conditions. Ex vivo conditions often allow methods, work, experimentation and/or measurements to be carious out under more controlled conditions than may be possible in in vivo experiments (in the intact organism), albeit at the expense of altering the “natural” environment. A main difference between in vitro and ex vivo assays is that the former is simply a cell system established in a cell culture or in a laboratory. In contrast, the latter is a tissue directly taken from a living organism, or a tissue that has been created artificially. The term “in vitro” (“within the glass,” or “in glass,” e.g., in a test tube or petri dish). In vitro experiments were historically conducted in glass test tubes and Petri dishes, however, modern laboratory practices include the use of single-use plasticware, and other non-glass instruments, in part to avoid cross-contamination, the need for washing, sterilization, etc. In vitro experiments are often performed on cells or cultures of cells, which sometimes include immortalized human or other animal cell lines, or other cell lines, and sometimes include bacteria. When the test subjects are cells, they are derived from living organisms or cell lines and maintained in culture in special conditions. Sometimes those conditions seek to mimic physiological circumstances including temperature and sterility. There is a wide variety of cell types which in many cases can be amplified in plastic flasks and distributed in plates with hundreds of small wells for high throughput screening, for example. Some cell types cannot adhere to plastic and are cultured in suspension, forming aggregates. Three-dimensional cell systems may also be used where the higher cell-cell interaction adds another layer of complexity to cellular communication. Furthermore, advanced in vitro systems such as the organ-on-a-chip technology, offer a next level of complexity introducing, for example, microfluidic channels that reproduce human blood and/or airflow. These are only some examples of in vitro methodologies. The activity of a connexin modulators, connexin hemichannel modulators, inflammasome modulators, inflammasome activation modulators, and pannexin channel modulators may be evaluated by in vitro, ex vivo and in vivo techniques, including those known in the art.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as a recorded presentation. As used herein, “instruction(s)” means documents or information describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as a recorded presentation. Instructions can comprise one or multiple documents, and include future updates. Kits may include one or more connexin modulators, one or more connexin hemichannel modulators, one or more inflammasome modulators, one or more inflammasome activation modulators, and/or one or more pannexin channel modulators, alone or in any combination.

The term “fatigue” refers to a feeling of weariness, tiredness, and/or lack of energy. Fatigue reduces energy, the ability to do things and the ability to focus. Ongoing fatigue can affect quality of life and state of mind. Fatigue can be mental or physical. It is often classified into physical fatigue, such as muscle fatigue, and mental fatigue, such as brain and sensory system fatigue. The inventions described and claimed herein can treat mental fatigue. The inventions described and claimed herein can also help to treat physical fatigue. Fatigue, which often refers to weariness, a lack of energy and/or motivation is different from drowsiness, which often refers to feeling the need to sleep. Drowsiness and apathy (a feeling of not caring about what happens), however, can be symptoms that go along with fatigue. In the present disclosure, “fatigue” can also include “loss of focus.” Fatigue is often characterized by fatigue that is not relieved with rest and is also often associated with physical symptoms. Fatigue can also be determined by evaluating its length and associated physical symptoms (including one or more of, for example, post-exertional malaise; unrefreshing sleep; impaired memory or concentration; muscle pain; polyarthralgia; sore throat; tender lymph nodes; or new headaches). Fatigue may also be determined and evaluate using an electroencephalogram (EEG) device known in the art (e.g., a device comprising a dry-contact EEG electrode to measure EEG data operably coupled to at least one processor to calculate a frequency domain representation of the EEG data, detect spectral features indicative of fatigue based on the frequency domain representation, and determining whether the brain is fatigued based on the detection). Compounds, compositions, kits and methods described herein may be used to treat fatigue resulting from a post-acute infection syndrome (e.g., long COVID, long flu, post-treatment Lyme disease syndrome, etc.).

Common signs/symptoms of “brain fog,” also known as mental fog or a clouding of consciousness, can include one or more of the following: difficulty concentrating, irritable mood, low energy or fatigue, headaches, trouble recalling information, anxious mood, confusion, and insomnia. Brain fog may be determined by medical or other evaluation. Diagnostic blood testing and medical imaging, such as MRI, are also sometimes used in the diagnosis of brain fog. Compounds, compositions, kits and methods described herein may be used to treat brain fog resulting from a post-acute infection syndrome (e.g., long COVID, long flu, post-treatment Lyme disease syndrome, etc.).

Other post-acute infection syndrome symptoms, including for example, joint pain, sleep disturbance, anxiety, headache, and other symptoms described or referenced herein or known in the art, including those set forth in the Example, may also be treated with compounds, compositions, kits and methods described herein (e.g. connexin 43 hemichannel modulators, NLRP3 inflammasome modulators, etc.). Symptoms of a post-acute infection syndrome may be determined, diagnosed or evaluating using methods known in the art, including differential diagnosis.

With regard to long COVID, for example, a detailed clinical history regarding the onset and duration of current symptoms, underlying medical comorbidities, the severity of COVID-19, and medication history is obtained by treating providers during follow-up visits and hospitalization records of COVID-19 related admission, if any, are reviewed thoroughly, including pertinent diagnostic tests performed. Given post-acute COVID-19 syndrome is an evolving clinical entity, currently, there are no guidelines regarding its management. Nevertheless, until further guidance is available, this novel clinical entity is be considered a diagnosis of exclusion, with other complications associated with COVID-19 and other acute alternative diagnoses being ruled out with, for example, pertinent laboratory and radiologic assessment, including reinfection with SARS-CoV-2. See Chippa V, et al. Post-Acute Coronavirus (COVID-19) Syndrome. [Updated 2023 Feb. 3]. In: StatPearls [Internet]. Treasure Island (FL): StatPearls Publishing; 2024 January-, and the descriptions of tests and assessments noted therein, which may include routine laboratory assessments, pulmonary imaging, cardiac function tests such as EKG and echocardiography, neuroimaging that includes vascular (CT/MRI) and neuropsychological testing if clinically indicated in patients presenting with neuropsychiatric manifestations.

As used herein, the term “modulator” refers to connexin hemichannel modulators, a pannexin channel modulators, direct inflammasome modulators and indirect inflammasome modulators. A modulator may refer to at least one, any two or more, any three or more, or all compounds and compositions selected from the group consisting of connexin hemichannel modulators, a pannexin channel modulators, direct inflammasome modulators and indirect inflammasome modulators.

As used herein, the terms “optional” or “optionally” means that the subsequently described event or circumstance can or cannot occur, and that the description includes instances where said event or circumstance occurs and instances where it does not.

As used herein, the term “pannexin channel modulator” (which may also be referred to as a pannexin channel or pannexin “inhibitor,” “blocker” or “antagonist”) is a compound that prevents, inhibits, and/or reduces the formation, function or activity of a pannexin channel, including, for example, the prevention, inhibition and/or reduction, in whole or in part, in the expression of a pannexin protein, its trafficking and/or assembly as a channel. Pannexin channel modulators include chemical-based pannexin channel modulators (e.g. probenecid, or a compound according to Formula VI in U.S. Pat. No. 10,465,188, etc.), peptide-based pannexin hemichannel modulators (e.g. ¹⁰Panx1), DNA- and RNA-based pannexin channel modulators, antibody- and antibody fragment-based pannexin channel modulators (e.g. monoclonal antibodies, ScFvs, etc.). See, e.g., Koval, M, et al. Pharmacology of pannexin channels. Current Opinion in Pharmacology 2023 69:102359; Navis, K E, et al. Pannexin 1 Channels as a Therapeutic Target: Structure, Inhibition, and Outlook ACS Chem. Neurosci. 2020, 11, 15, 2163-2172; Willebrords J, et al. Inhibitors of connexin and pannexin channels as potential therapeutics. Pharmacol Ther. 2017 180:144-160. In some embodiments, the pannexin channel modulators are pannexin 1 (Panx1) channel modulators. Pannexin modulation (e.g., pannexin 1 modulation) will be useful in treating or preventing one or more of the diseases, defects, disorders or conditions described herein by inhibiting ATP release from a pannexin channel (e.g., a pannexin 1 channel).

References in the specification and claims to parts by weight of a particular agent or component in a composition denotes the weight relationship between the agent or component and any other agents or components in the composition or article for which a part by weight is expressed. Thus, in a compound or composition containing 2 parts by weight of component X and 5 parts by weight component Y, X and Y are present at a weight ratio of 2:5, and are present in such ratio regardless of whether additional components are contained in the compound. A weight percent (wt. %) of a component, unless specifically stated to the contrary, is based on the total weight of the formulation or composition in which the component is included. Thus, the amount of X in a compound or composition containing X (5 wt. %) contains 5% X.

Peptidomimetics are molecules able to mimic natural peptides and proteins. Generally, peptidomimetics are structural or functional mimics (e.g., identical or similar) to a paradigm polypeptide (i.e., a polypeptide that has a biological or pharmacological function or activity). The terms “peptidomimetic” and “mimetic” (also known or referred to as peptide mimetics) include synthetic or recombinantly manufactured compounds, including polyamides and poly(amino acid) polymers, that have the same or substantially the same characteristics of protein or peptide regions that they mimic. Typically, a peptidomimetic will have the same or substantially the same structural and/or functional characteristics of protein or peptide regions they mimic. A peptidomimetic modulator (also sometimes referred to as a “modulator peptide,” a “modulator peptidomimetic,” a “mimetic” or a “peptidomimetic inhibitor”) comprises a peptide or peptide-like molecule that is able to serve as a model for a peptide substrate upon which it is structurally based and to modulate (i.e., inhibit, lessen, decrease or reduce) one or more activities of the peptide substrate on which is structurally based.

In some embodiments, the peptidomimetics and mimetics comprise one or more or all naturally occurring amino acids. In some embodiments, the peptidomimetics and mimetics comprise one or more or all L-amino acids. In some embodiments, the peptidomimetics and mimetics comprise one or more or all D-amino acids. D-peptide peptidomimetics include D-peptides designed to mimic natural L-peptides. The peptide bonds formed by D-amino acids are more resistant to proteases than those formed by L-amino acids. In some embodiments, the peptidomimetics and mimetics comprise one or more L-amino acids and one or more D-amino acids. In some embodiments, a peptidomimetic can be a mimetic composed entirely of natural amino acids, composed entirely synthetic chemical compounds, composed entirely of non-natural amino acids, composed entirely of analogues of naturally-occurring and/or non-naturally-occurring amino acids, or a chimeric molecule composed of partly of natural amino acids and partly of non-natural occurring amino acids or analogs of amino acids. In some embodiments, a peptidomimetic comprises any amount of natural amino acid conservative substitutions as long as such substitutions also do not substantially alter mimetic activity. In some embodiments, peptidomimetics are unmodified.

In some embodiments, peptidomimetics are modified. In some embodiments, a peptidomimetic is a modified or chemically modified peptide. In some embodiments, peptidomimetics is a modified peptide that has one or more peptide linkages optionally replaced by a linkage selected from the group consisting of, for example, —CH₂NH—, —CH₂S—, —CH₂—CH₂—, —CH═CH— (cis and trans), —COCH₂—, —CH(OH)CH₂—, and —CH₂SO—. In some embodiments, a peptidomimetics is chemically modified to include unnatural amino acid substitutions, backbone amide bond modifications, rigid scaffolds, added hydrophobic residues, and/or in other ways known in the art. In some embodiments, a peptidomimetic is a modified peptide-like molecule. In some embodiments, a modified peptide-like peptidomimetic contains one or more or all non-naturally occurring amino acids or is a peptoid. Although their backbones are similar, side chains of peptides are attached to the α-carbon in peptides, while peptoids have side chains attached to the amide nitrogen. This modification confers resistance to proteolytic degradation. Peptoids include poly-N-substituted glycines. See, e.g., Wolf L M, et al. Peptoids: Emerging Therapeutics for Neurodegeneration J Neurol Neuromedicine 2017 2(7):1-5. Other peptidomimetics known in the art include, for example, peptide-like molecules which contain a constrained amino acid, a non-peptide component that mimics peptide secondary structure, or an amide bond isostere. See, e.g., Goodman and Ro, Peptidomimetics for Drug Design, in “Burger's Medicinal Chemistry and Drug Discovery” Vol. 1 (ed. M. E. Wolff, John Wiley & Sons 1995), pages 803-861. A peptidomimetic that contains a constrained amino acid or a constrained non-naturally occurring amino acid can include, for example, an α-methylated amino acid, an α,α-dialkyl-glycine or α-aminocycloalkane carboxylic acid; an Nα-Cα cylized amino acid, an Nα-methylated amino acid, a β- or γ-amino cycloalkane carboxylic acid, an α,β-unsaturated amino acid, a β, β-dimethyl or β-methyl amino acid, a β-substituted-2,3-methano amino acid, an NCδ or Cα-Cδ cyclized amino acid, or a substituted proline or another amino acid mimetic. A peptidomimetic that mimics peptide secondary structure can contain, for example, a nonpeptidic β-turn mimic; γ-turn mimic; mimic of β-sheet structure; or mimic of helical structure, each of which is well known in the art. A peptidomimetic that contains an amide bond isostere, for example, may contain an amide bond isostere such as a retro-inverso modification, a reduced amide bond, a methylenethioether or methylenesulfoxide bond, a methylene ether bond, an ethylene bond, a thioamide bond, a trans-olefin or fluoroolefin bond, a 1,5-disubstituted tetrazole ring, a ketomethylene or fluoroketomethylene bond, or another amide isostere. One skilled in the art understands that these and other peptidomimetics are encompassed within the meaning of the term “peptidomimetic” as used herein.

Peptidomimetics that are structurally similar to portions of a connexin (e.g., connexin 43), an inflammasome (e.g., an NLRP3 inflammasome) or a pannexin (e.g., pannexin 1) can be used to produce a therapeutic or prophylactic effect.

Peptidomimetics useful as connexin hemichannel modulating agents include peptide and peptide-like molecules capable of down-regulating, reducing or decreasing, in whole or in part, one or more biological actions or activities of a connexin hemichannel, such as, for example, preventing the docking of hemichannels to form gap-junction-mediated cell-cell communications, or preventing the opening of hemichannels to expose the cell cytoplasm to the extracellular millieu, and so on.

By way of example, in some embodiments, a peptidomimetic mimics an extracellular loop of connexins involved in hemichannel-hemichannel docking and cell-cell channel formation, and/or an extracellular loop of a connexin hemichannel, as well as an extracellular loop or the intracellular loop or intracellular C-terminus of a connexin. In some embodiments, a peptidomimetic mimics an extracellular loop, intracellular loop or intracellular C-terminus of connexin 43. In some embodiments, a peptidomimetic mimics an portion of another connexin, including those listed herein.

Compositions of the invention may comprise one or more connexin peptidomimetics that can serve as connexin hemichannel modulators (also referred to “mimetic connexin hemichannel modulators” or “peptidomimetic connexin hemichannel modulators”). Connexin peptidomimetics are known in the art and many connexin peptidomimetics useful as peptidomimetic connexin hemichannel modulators are described or referenced herein. All known connexin isoforms are structurally similar, being composed of four transmembrane domains, two extracellular loops (EL1, EL2) bound in conformation by extracellular disulfide bonds, a single intracellular loop (IL), an amino-terminus (NT), and a carboxyl-terminus (CT). Connexin peptidomimetics useful as connexin hemichannel modulators include, for example, EL1 extracellular loop-binding peptidomimetics such as Gap 26 and P5, EL1 extracellular loop-binding peptidomimetics such as Peptide5, Gap 27, and P180-195, IL intracellular loop-binding peptidomimetics such as Gap19, Gap20 and Cx43L2 peptide, and intracellular CT-binding peptidomimetic connexin hemichannels modulators such as XG19, JM2, cSRC, TAT-Cx43, TAT-CT10, and the alpha CT peptidomimetics such as alpha CT-1, alpha CT-2, alpha CT-3, etc. See, e.g., King D R, et al. Mechanisms of Connexin Regulating Peptides. Int J Mol Sci. 2021 22(19):10186: Simon, Á, et al. Peptide Binding Sites of Connexin Proteins. Chemistry 2020, 2:662-673. FDA-approved peptidomimetics drugs include Romidepsin (Istodax), Atazanavir (Reyataz), Saquinavir (Invirase), Oktreotid (Sandostatin), Lanreotide (Somatuline), Plecanatide (Trulance), Ximelagatran (Exanta), Etelcalcetide (Parsabiv), and Bortezomib (Velcade).

The first extracellular loops of connexin 37, connexin 40 as well as connexin 43 are mimicked by peptides under the Gap26 code. Gap27 and Peptide5, on the other hand, mimic regions of the second extracellular loop. Gap27 targets connexin 32, connexin 40 and connexin 43, while Peptide5 is used for connexin 43 inhibition. Other peptidomimetics include JM2 (VFFK-GVKDRVKGRSD; SEQ ID NO:134), ASH3, CT9 (RPRDDLEI; SEQ ID NO:135) and CT9-TAT, CT10 (SRPRDDLEI; SEQ ID NO:136), aCT (RQPKIWFPNRRKPWKK-RPRPDDLEI (SEQ ID NO:137) in which the inhibitor peptide includes the connexin 43 C-terminal amino acids 374-382 (RPRPDDLEI; SEQ ID NO:141) that encompass the ZO-1-binding sequence, which is in turn attached at its N-terminus to a 16-amino acid antennapedia internalization vector) mimic the C-terminal tail of connexin 43 and Gap24 (GHGDPLHLEEVKC; SEQ ID NO:138) reproduces a sequence of the cytoplasmic loop of connexin 32 while TAT-Gap24 (YGRKKRRQRRRGHGDPLHLEEVKC; SEQ ID NO:139) also mimics a portion of the intracellular loop of connexin 43. Both L2 and Gap19 also mimic the cytoplasmic loop of connexin 43. Peptides and peptidomimetics useful in the compositions and methods of the invention also include those set forth in Tables II and III. In some embodiments, a peptidomimetic connexin hemichannel modulator has been modified to increase stability, to improve bioavailability and/or to increase cell membrane permeability, for example, by linking the amino or carboxy terminus to a cellular internalization transporter. Cellular internalization transporters include those described herein or otherwise known in the art or later developed. In some embodiments, a peptidomimetic corresponds to at least one portion of a connexin (e.g., connexin 43) that can be used to modulate a connexin hemichannel activity (including, for example, release of ATP).

In some embodiments, a peptidomimetic corresponds to at least one portion of an inflammasome (e.g., an NLRP3 inflammasome). In some embodiments, by way of example, an NLRP3 inflammasome peptidomimetic comprises a sequence corresponding to segment of an NLRP3 inflammasome component (e.g., an amino (N)-terminal pyrin domain (PYD), a carboxy (C)-terminal LRR, or a central NBD-containing ATPase domain (NACHT)), or to an apoptosis-associated speck-like protein containing a CARD (ASC). Inhibitors constituents of the NLRP3 Inflammasome include VX-740 (Pralnacasan) and its analog VX-765, which are peptidomimetic inhibitors of caspase-1.

Small-molecule inflammasome modulators useful in methods of the invention the orally bioavailable proteasome inhibitor NIC-0102 (IUPAC/Chemical Name: ((R)-1-((S)-2-(2,6-difluorobenzamido)-3-phenylpropanamido)-3-methylbutyl)boronic acid). NIC-102 specifically prevents NLRP3 inflammasome activation but has no effect on NLRC4 or AIM2 inflammasomes. NIC-0102 induces the polyubiquitination of NLRP3, interferes with the NLRP3-ASC interaction, and blockes ASC oligomerization, thereby inhibiting NLRP3 inflammasome activation. In addition, NIC-0102 also inhibits the production of pro-IL-10.

In some embodiments, a peptidomimetic corresponds to at least one portion of a pannexin (e.g., pannexin 1) to modulate pannexin channel activity (including, for example, release of ATP). In some embodiments, the pannexin 1 peptidomimetic comprises or consists essentially of H-Trp-Arg-GIn-Ala-Ala-Phe-Val-Asp-Ser-Tyr-OH (also known as ¹⁰Panx).

Peptidomimetics encompass those described herein, as well as those as may be known in the art, whether now known or later developed.

The term “pharmaceutical formulation” refers to a preparation which is in such form as to permit the biological activity of an active ingredient contained therein to be effective, and which does not contain additional components that are unacceptably toxic to a subject to which the formulation would be administered. A “pharmaceutical composition” refers to a mixture of substances suitable for administering to a subject that includes one or more active ingredients or pharmaceutical agents (e.g., one or more connexin hemichannel modulators (e.g., one or more connexin 43 hemichannel modulators), one or more inflammasome modulators (e.g., one or more NLRP3 inflammasome modulators) or a combination of one or more connexin hemichannel modulator and one or more inflammasome modulators). For example, a pharmaceutical composition may comprise one or more compounds of the invention and a sterile aqueous solution or a pharmaceutically acceptable carrier.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative.

As used herein, the term “prevent” or “preventing” refers to precluding, averting, obviating, forestalling, stopping, or hindering something from happening. It is understood that where reduce, inhibit or prevent are used herein, unless specifically indicated otherwise, the use of the other two words is also expressly disclosed, and that they refer to reducing, inhibiting or preventing in whole or in part for the therapeutic and/or prophylactic purposes described herein.

Prodrugs are well known to those in the art with knowledge of chemistry. As used herein, a “prodrug” is understood to refer to a compound that, after administration, is metabolized or otherwise converted to an active or more active form with respect to at least one biological property, relative to itself. To produce a prodrug, a pharmaceutically active compound (e.g., tonabersat), or a suitable precursor thereof, is modified chemically such that the modified form is less active or inactive, but the chemical modification is effectively reversible under certain biological conditions such that a pharmaceutically active form of the compound is generated by metabolic or other biological processes. A prodrug can have, relative to the drug, altered metabolic stability or transport characteristics, fewer side effects or lower toxicity, or improved flavor, for example (see the reference Nogrady, 1985, Medicinal Chemistry A Biochemical Approach, Oxford University Press, New York, pages 388-392). Prodrugs can also be prepared using compounds that are not drugs but which upon activation under certain biological conditions generate a pharmaceutically active compound. As used herein, for example, a “tonabersat prodrug” or “prodrug of tonabersat” refers to a compound that undergoes a chemical conversion, through a metabolic process or otherwise (e.g., within the body of the mammal or other subject receiving the compound) into an active form of tonabersat that has a desired effect. In other words, a tonabersat prodrug includes a compound that upon activation releases tonabersat or an active analog thereof.

A “small molecule” is a low molecular weight organic compound and gernally includes compounds with a molecular weight less than about 600-1200 daltons. Small molecule connexin modulators include the compounds of Formula I. A small molecule can be an active agent of a prodrug. Small molecules prodrugs include the compounds of Formula II, which are prodrugs of the small molecule connexin hemichannel modulator, tonabersat. The molecular weight of tonabersat is about 392 daltons. Weight average molar mass or weight average molecular weight is the ratio of the total mass of all molecules to the total number of moles in the sample. This value is also known as the average molecular weight, which for tonabersat is about 392 daltons, depending on the preparation and its purity. This is noted because infringers have in the past used values for molecular weight versus average molecular weight to confuse and deceive courts into believing, for example, that the compound it commercializes that is identical to a claimed compound, and thus infringes the claim, is not the claimed compound because the “molecular weight” of the compound referred to in the claim is different from the “average molecular weight” of the infringing compound.

As used herein, the term “subject” or the like, including “individual,” and “patient”, all of which may be used interchangeably herein, refers to any mammal, including humans, domestic and farm animals, and zoo, wild animal park, sports, or pet animals, such as dogs, horses, cats, sheep, pigs, cows, etc. In some instances, however, a patient may refer to a subject afflicted with specific a condition, disease, defect, syndrome or disorder. The terms “subject” and “patient” include human and veterinary subjects. The preferred mammal herein is a human, including adults, children, and the elderly. Preferred sports animals are horses and dogs. Preferred pet animals are dogs and cats. The subject may be, for example, an aquatic park animal, such as a dolphin, whale, seal or walrus. In certain embodiments, the subject, individual or patient is a human. The term “subject” does not denote a particular age or sex. In some embodiments, subjects may include animals used in scientific experiments (e.g. mice, rats, rabbits, sheep, goats, or other laboratory subjects). In other embodiments, the term “subjects” may exclude one or more or all animals used in scientific experiments, and a method may specify that is does not include one or more or all animals used in scientific experiments. In some embodiments, subjects may include non-animals and other things used in scientific experiments (e.g., fruit flies, cells, cell cultures, tissues, organs, 3D tissue culture (such as organs-on-a-chip). In other embodiments, the term “subjects” may exclude one or more or all non-animals or other things used in scientific experiments and a method may specify that is does not include one or more or all non-animals or other things used in scientific experiments.

In some embodiments of the disclosed methods, a subject has been diagnosed with a need for treatment of one or more signs and/or symptoms associated with a post-acute infection syndrome.

As used herein, “treatment” (and grammatical variations thereof such as “treat” or “treating”) refers to an attempt to alter the course of the individual, tissue or cell being treated, and can be performed either for prophylaxis or during the course of clinical pathology. Thus, the term “treatment” includes the management or care of a subject, or of a patient, with the intent to cure, ameliorate, stabilize, or prevent a disease, disorder, defect or condition. This term includes active treatment, that is, treatment directed specifically toward the improvement of a disease, disorder, defect or condition, and also includes causal treatment, that is, treatment directed toward removal of the cause of the associated disease, disorder, defect or condition. In addition, this term includes palliative treatment, that is, treatment designed for the relief of symptoms rather than the curing of the disease, disorder, defect or condition; preventative treatment, that is, treatment directed to minimizing or partially or completely inhibiting the development of the associated disease, disorder, defect or condition; and supportive treatment, that is, treatment employed to supplement another specific therapy directed toward the improvement of the associated disease, disorder, defect or condition. In various aspects, the term covers any treatment of a subject, including a mammal (e.g., a human), and includes: (i) preventing a disease, disorder or condition from occurring in a subject that can be predisposed to the disease, disorder or condition but has not yet been diagnosed as having it; (ii) inhibiting a disease, disorder or condition, i.e., arresting or slowing its development; or (iii) relieving a disease, disorder or condition, i.e., causing regression of the disease, disorder or condition. In some embodiments, the subject is a mammal such as a primate, and, in further embodiments, the subject is a human. In some embodiments, the subject is a non-human mammal. Treatment does not necessarily imply that a subject or patient is treated until total recovery following treatment, or that treatment of a subject or patient results in total recovery. Accordingly, “treatment” may also include maintaining or promoting a complete or partial state of remission in a subject or patient, or an alleviation or relief from symptoms of the disease, disorder, defect of conditions being treated, whether in whole or in part, temporary or permanent. The term “preventing” means preventing in whole or in part or ameliorating or controlling. The term “ameliorate,” “ameliorating” and “amelioration” and the like refer an improvement in one or more signs or symptoms of a post-acute infection syndrome or condition, a reduction in the severity of one or more signs or symptoms of a post-acute infection syndrome or condition, or an inhibition of progression or worsening of one or more signs or symptoms of a post-acute infection syndrome or disorder (e.g., stabilizing one or more signs or symptoms of a post-acute infection syndrome or disorder). In the case of long COVID, long flu, long cold, post-Lyme disease treatment syndrome, etc., an improvement can be a lowering or a reduction in one or more signs or symptoms or effects of the post-acute infection syndrome disorder.

In some embodiments, “treat,” “treating”, or “treatment” means the administration of therapy to an individual who already manifests at least one sign or symptom of a post-acute infection syndrome, disease or condition or who has previously manifested at least one sign or symptom of a post-acute infection syndrome, disease or condition. For example, “treating” can include alleviating, abating or ameliorating at least one sign or symptom of a post-acute infection syndrome, disease or condition, preventing additional one or more signs and/or symptoms, ameliorating an underlying cause of one or more signs and/or symptoms, inhibiting at least one sign or symptom of a post-acute infection syndrome, disease or condition, e.g., arresting the development of the syndrome, and/or relieving at least one sign or symptom of a post-acute infection syndrome, disease or condition, causing regression of at least one sign or symptom of a post-acute infection syndrome, disease or condition, relieving at least one sign and/or symptom caused by a post-acute infection syndrome, disease or condition, or stopping at least one sign and/or symptom caused by a post-acute infection syndrome, disease or condition. For example, the term “treating” in reference to a post-acute infection syndrome, disorder, disease or condition, means a reduction in severity of one or more signs and/of symptoms associated with that particular post-acute infection syndrome, disorder, disease or condition. Therefore, treating a post-acute infection syndrome, disorder, disease or condition does not necessarily mean a reduction in severity of all symptoms associated with a post-acute infection syndrome, disorder, disease or condition and does not necessarily mean a complete reduction in the severity of one or more signs and/or symptoms associated with a post-acute infection syndrome, disorder, disease or condition.

In some embodiments, compounds, compositions, methods, and kits of the invention are used in the treatment of a subject or a patient for one or more signs and/or symptoms of a post-acute infection syndrome.

In some embodiments, compounds, compositions, methods, and kits of the invention are used alleviation of one or more signs or symptoms of, or diminishment of one or more direct or indirect pathological consequences of a post-acute infection syndrome.

Any modulator that is capable of eliciting a desired inhibition of the passage (e.g., transport) of molecules through a connexin gap junction and/or connexin hemichannel (e.g., ATP), may be used in embodiments of the invention and dosed according to the one or more of the schedules described herein. Any connexin agents that modulates the passage of molecules through a gap junction or connexin hemichannel (e.g., a connexin 43 gap junction or connexin 43 hemichannel) are also provided in particular embodiments (e.g., those that modulate, block or lessen the passage of molecules from the cytoplasm of a cell into an extracellular space or adjoining cell cytoplasm, including ATP). Such anti-connexin modulators may modulate the passage of molecules through a gap junction or connexin hemichannel with or without gap junction uncoupling (blocking the transport of molecules through gap junctions). Such compounds include, for example, binding proteins (e.g. scFvs, antibodies, etc.), polypeptides (e.g. peptidomimetics), polynucleotides (e.g., antisense compounds) and organic compounds (e.g. tonabersat and compounds of Formula I and/or prodrugs thereof, including the tonabersat prodrugs of Formula II) that can, for example, block the function or activity of a gap junction or a hemichannel in whole or in part (e.g. by modulating release of ATP from connexin hemichannels).

### Chemical Connexin Hemichannel Modulators

In some embodiments, the modulator used in methods of the invention is a gap junction closing or blocking compound or hemichannel closing or blocking compound (e.g. tonabersat). In some embodiments, the modulator can be a small molecule, which may also be referred to herein as an anti-connexin or a connexin or connexin gap junction or connexin hemichannel modulator. In some embodiments, methods of the invention feature the use of compounds of Formula I, for example tonabersat and/or carabersat, to directly and immediately block Cx43 hemichannels and to cause a concentration and time-dependent reduction in GJ coupling and/or hemichannel inhibition (e.g., blocking hemichannel opening and/or modulating or blocking ATP release from connexin hemichannels). Carabersat is N-[(3R,4S)-6-acetyl-3-hydroxy-2,2-dimethyl-3,4-dihydrochromen-4-yl]-4-fluorobenzamide) and is also referred to as trans-(+)-6-acetyl-4-(S)-(4-fluorobenzoylamino)-3,4-dihydro-2,2-dimethyl-2H-1-benzo[b]pyran-3R-ol,hemihydrate. Tonabersat is also known by the IUPAC name N-[(3S,4S)-6-acetyl-3-hydroxy-2,2-dimethyl-3,4-dihydrochromen-4-yl]-3-chloro-4-fluorobenzamide or (3S-cis)-N-(6-acetyl-3,4-dihydro-3-hydroxy-2,2-(dimethyl-d6)-2H-1-benzopyran-4-yl)-3-chloro-4-fluorobenzamide).

In some embodiments, the anti-connexin or connexin or connexin gap junction or connexin hemichannel modulator is a compound according to Formula I:

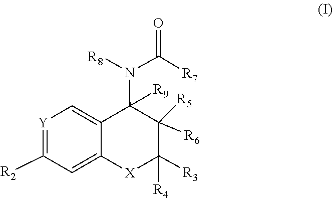

wherein Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃—S—; or a group CF₃-A-, where A is —CF₂—, —CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl;
one of R₃ and R₄ is hydrogen or C₁₋₄ alkyl and the other is C₁₋₄ alkyl, CF₃, or CH₂X^{a}, where X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁. 4 alkyl;
R₅ is C₁₋₆ alkylcarbonyloxy, benzoyloxy, ONO₂, benzyloxy, phenyloxy or C₁. 6 alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein Rₙ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino, amino-C₁₋₆ alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆ alkyl, aryl or heteroaryl; the R₈—N—CO—R₇ group being cis to the R₅ group; and
X is oxygen or NR₁₂ where R₁₂ is hydrogen or C₁₋₆alkyl.

For any of the Markush groups set forth above, in some embodiments, each group can include or exclude any of the species listed for that group.

In some embodiments, the small molecule connexin modulator can be Tonabersat, carabersat, or SB-204269. SB-204269 is also known as (trans-(+)-6-acetyl-4S-(4-fluorobenzoylamino)-3, 4-dihydro-2,2-dimethyl-2H-benzo[b]pyran-3R-ol). Carabersat is also known as N-[(3R,4S)-6-Acetyl-3-hydroxy-2,2-dimethyl-3,4-dihydro-2H-chromen-4-yl]-4-fluorobenzamide. Tonabersat is also known as N-(6-Acetyl-3-hydroxy-2,2-dimethyl-3,4-dihydro-2H-chromen-4-yl)-3-chloro-4-fluorobenzamide.

For any of the Markush groups set forth above, that group can include or exclude any of the species listed for that group.

In some embodiments, the modulator may be a prodrug of any the compounds for use in this invention. In one aspect the connexin modulator prodrug of this invention may be a compound of Formula II:

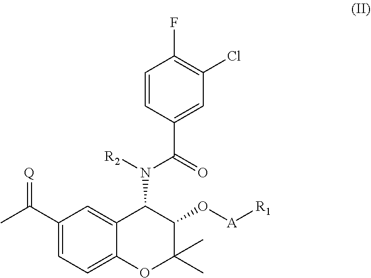

wherein
Q is O or an oxime,
R₂ is H,
A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁,
R₃ and R₄ are selected independently from H, fluoro, C₁₋₄ alkyl, and C₁₋₄ fluoroalkyl, or R₃ and R₄ together with the atom to which they are attached form a cyclopropyl group,
R₁ is selected from groups [1], [2], [2A],[3], [4], [5] and [6] wherein the atom marked ** is directly connected to A:

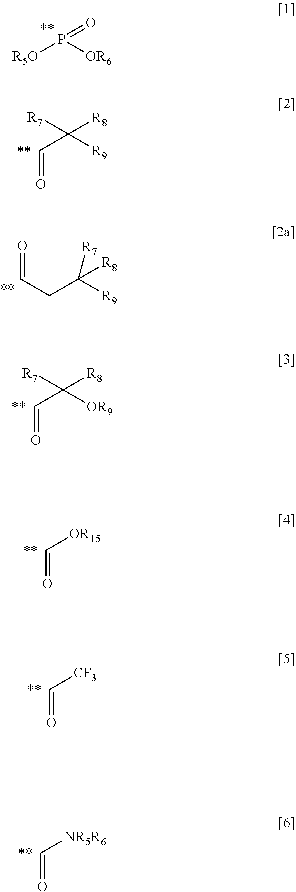

Wherein R₅ and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(i) H, C₁₋₄ alkyl, or C₁₋₄ fluoroalkyl,
(ii) the side chain of a natural or unnatural amino acid or peptide as described herein, and
(iii) biotin or chemically linked to biotin;
R₉ is selected from H, —N(R₁₁)(R₁₂), —N⁺(R₁₁)(R₁₂)(R₁₃)X⁻, and —N(R₁₁)C(O)R₁₄;
wherein R₁₁, R₁₂, and R₁₃ are independently selected from H, C₁₋₄ alkyl, and C₁₋₄ fluoroalkyl,
R₁₄ is H, C₁₋₄ alkyl, or C₁₋₄ fluoroalkyl,
R₁₅ is selected from C₁₋₄ alkyl and C₁₋₄ fluoroalkyl, and
X⁻ is a pharmaceutically acceptable anion.

In some aspects,
R₂ is B—R₂₁ wherein,
B is a direct bond, —C(O)O*—, —C(R₂₃)(R₂₄)O*, C(O)OC(R₂₃)(R₂₄)*, or —C(R₂₃)(R₂₄)OC(O)O* wherein the atom marked * is directly connected to R₂₁, R₂₃ and R₂₄ are selected independently from H, fluoro, C₁₋₄ alkyl, and C₁₋₄ fluoroalkyl, R₂₁ is selected from groups [21], [22], [22A], [23], [24], [25] and [26] wherein the atom marked ** is directly connected to B:

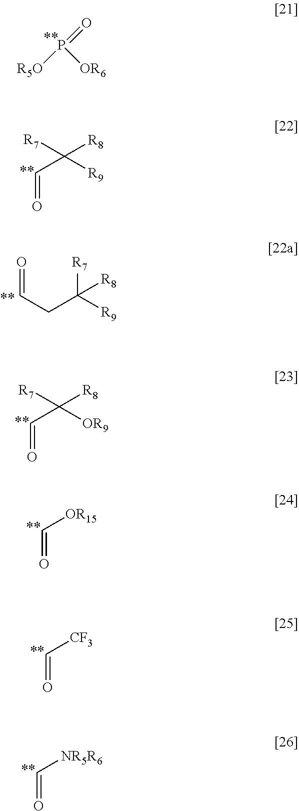

wherein R₅, R₆, R₇, R₈, R₉, and R₁₅ are as defined herein.

For any of the Formula II Markush groups set forth above, that group can include or exclude any of the species listed for that group.

In some aspects, the peptide as described herein can be a connexin modulator, calmodulin modulator, or pannexin modulator.

In some aspects,
Q is an oxime of formula ═NOR₄₃, wherein R₄₃ is
(i) selected from H, C₁₋₄ fluoroalkyl or optionally substituted C₁₋₄ alkyl, and
(ii) -A₃₀₀-R₃₀₀ wherein
A₃₀₀ is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₃₀₀,
R₃ and R₄ are selected independently from H, fluoro, C₁₋₄ alkyl, and C₁₋₄ fluoroalkyl, or R₃ and R₄ together with the atom to which they are attached form a cyclopropyl group,
R₃₀₀ is selected from groups [1], [2], [2A], [3], [4], [5] and [6] wherein the atom marked ** is directly connected to A₃₀₀:

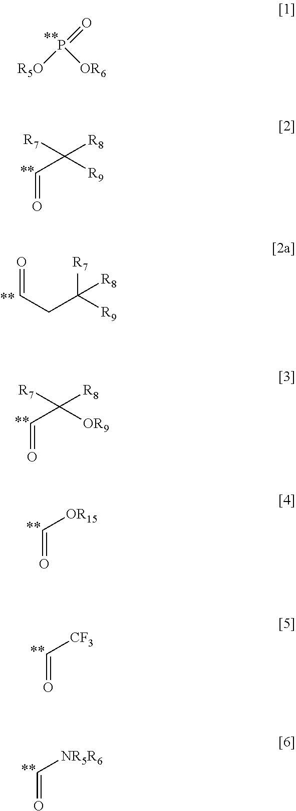

wherein R₅ and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(i) H, C₁₋₄ alkyl, or C₁₋₄ fluoroalkyl,
(ii) the side chain of a natural alpha-amino acid, and
(iii) biotin or chemically linked to biotin;
R₉ is selected from H, —N(R₁₁)(R₁₂), —N⁺(R₁₁)(R₁₂)(R₁₃)X⁻, and —N(R₁₁)C(O)R₁₄;
wherein R₁₁, R₁₂, and R₁₃ are independently selected from H, C₁₋₄ alkyl, and C₁₋₄ fluoroalkyl,
R₁₄ is H, C₁₋₄ alkyl, or C₁₋₄ fluoroalkyl,
R₁₅ is selected from C₁₋₄ alkyl and C₁₋₄ fluoroalkyl, and
X⁻ is a pharmaceutically acceptable anion.

In an embodiment R₄₃ is C₁₋₄ alkyl optionally substituted with a phosphate group (P(O)OR₆₁R₆₂). In an example of such an embodiment OR₄₃ is —OCH₂P(O)OR₆₁OR₆₂, wherein R₆₁ and R₆₂ are independently H or C₁₋₄ alkyl.

In another embodiment of Formula II R₄₃ is an amino acid derivative having the structure C(O)CH(R₁₀₀)NH₂ wherein the group R₁₀₀ is the side chain of a natural or unnatural amino acid or a peptide as described herein.

In one embodiment of Formula II R₈ is selected from:
(i) H, C₁₋₄ alkyl, or C₁₋₄ fluoroalkyl,
(ii) the side chain of a natural alpha-amino acid, and
(iii) biotin or chemically linked to biotin.

In some embodiments, the natural amino acid is selected from one of the 22 canonical amino acids. In some embodiments, the unnatural amino acid is selected from any amino acid which is not one of the 22 canonical amino acids. In some embodiments, the unnatural amino acid is selected from: (cis)-3-Aminobicyclo[2.2.1]heptane-2-carboxylic acid hydrochloride, exo-cis-3-Aminobicyclo[2.2.1]hept-5-ene-2-carboxylic acid hydrochloride, cis-2-Amino-2-methylcyclohexanecarboxylic acid hydrochloride, (R)-2-(Boc-amino)octanedioic acid, Boc-4-(Fmoc-amino)-L-phenylalanine, Boc-(2-indanyl)-Gly-OH, (R)-4-Boc-3-morpholineacetic acid, (S)-4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3,5-F2)-OH, 2-(4-Boc-piperazino)-2-(2-fluorophenyl)acetic acid, 2-(4-Boc-piperazino)-2-(4-fluorophenyl)acetic acid, 2-(4-Boc-piperazino)-2-phenylacetic acid, 2-(4-Boc-piperazino)-2-(3-pyridyl)acetic acid, penicillamine, thialysine, quisqualic acid, canavanine, azetidine-2-carboxylic acid, Carboxyglutamic acid, Hydroxyproline, Hypusine, and Pyroglutamic acid.

In one embodiment of Formula II OR₄₃ is —OC(O)CH(CH(CH₃)₂)NH₂.

For any of the Formula II Markush groups set forth above, in some embodiments, each group can include or exclude any of the species listed for that group.

In some embodiments “promoiety” refers to a species acting as a protecting group which masks a functional group within an active agent, thereby converting the active agent into a pro-drug. The active agent may be any of the modulators or ocular therapeutics disclosed herein. Typically, the promoiety will be attached to the drug via bond(s) that are cleaved by enzymatic or non-enzymatic means in vivo, thereby converting the pro-drug into its active form. In some embodiments the promoiety may also be an active agent. In some embodiments the promoiety may be bound to a connexin modulator, a connexin gap junction modulator, or a connexin hemichannel modulator. In some embodiments the promoiety may be bound to any of the polynucleotides, peptides or peptidomimetics, small molecule antagonists and/or other treatments disclosed herein. In some embodiments the promoeity may be bound to a compound of Formula I. In some embodiments the pro-drug may be a compound of Formula II.

In some embodiments the promoiety may be any peptidomimetic or peptide antagonist of this disclosure. In some embodiments, the promoeity is a single amino acid which is optionally protected on its functional groups. In some embodiments, the promoeity is a targeting species. In some embodiments, the promoeity is a substrate for an influx or efflux transporters on the cell membrane, for example those described in Gaudana, R. et al. The AAPS Journal, 12:3, 348-360 (2012). The promoeity can be, for example, chemically-linked biotin. The promoeity can be, for example, chemically-linked D-serine.

In some embodiments, compounds of Formula I or Formula II, e.g., tonabersat, carabersat or analogues thereof, are nonionic, are in the form of a free base, a free acid, or a pharmaceutically acceptable salt. By way of example, a pharmaceutically acceptable salt includes a hydrochloride salt and salts derived from acid including, but not limited to, hydrobromic acid, hydrochloric acid, phosphoric acid, acetic acid, fumaric acid, maleic acid, salicylic acid, citric acid, oxalic acid, lactic acid, malic acid, succinic acid, methanesulphonic acid and p-toluene sulphonic acid, a salt of itself. In one embodiment, the salt is a hydrochloride salt. In one embodiment, the salt is a succinate salt.

In other embodiments, one or more polymorph, one or more isomer, and/or one or more solvate of a compound of Formula I or Formula II, e.g., tonabersat, carabersat or analogues thereof, may be used.

### Peptidomimetic Connexin Hemichannel Modulators

In some embodiments, connexin modulators, including connexin, connexin gap junction and connexin hemichannel modulators, useful in methods of the invention include not only organic molecule-based connexin hemichannel modulators, but also but also connexin peptides or peptidomimetics, such as connexin 43 peptides or peptidomimetics, sometimes referred to anti-connexin peptides or peptidomimetics, e.g., anti-connexin hemichannel blocking peptides or peptidomimetics, which may be used in methods of the invention and dosed according to the one or more of the schedules described herein. They include, for example, peptides or peptidomimetic connexin modulators comprising or consisting essentially of sequences corresponding to portions of connexin extracellular domains, connexin transmembrane regions, and connexin carboxy-terminus regions. In some embodiments, connexin, connexin gap junction and connexin hemichannel modulators useful in methods of the invention include connexin 43 peptides or peptidomimetics with sequences according to a portion of the amino acid sequence of SEQ ID NO:100 (connexin 43 protein).

In some embodiments, the connexin modulators useful in methods of the invention for treating a disease, disorder, defect in a subject are anti-connexin 43 peptides or peptidomimetics. In some embodiments, anti-connexin 43 peptides or peptidomimetic that may be used in methods of the invention and dosed according to the one or more of the regemins described herein is a peptide comprising or consisting essentially of a portion of an extracellular domain of a connexin (e.g. connexin 43) and/or a peptide comprising or consisting essentially of a portion of a carboxy-terminal portion of a connexin (e.g. connexin 43), including those described and/or referenced herein.

The protein sequence of connexin 43 is shown below.

```
Connexin 43
(SEQ ID NO: 100)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
              20 25 30
Arg Ile Leu Leu Leu Gly Thr Ala Val Glu Ser Ala Trp Gly Asp Glu
        35 40 45
Gln Ser Ala Phe Arg Cys Asn Thr Gln Gln Pro Gly Cys Glu Asn Val
    50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                    85 90 95
Phe Tyr Val Met Arg Lys Glu Glu Lys Leu Asn Lys Lys Glu Glu Glu
              100 105 110
Leu Lys Val Ala Gln Thr Asp Gly Val Asn Val Asp Met His Leu Lys
          115 120 125
Gln Ile Glu Ile Lys Lys Phe Lys Tyr Gly Ile Glu Glu His Gly Lys
    130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                    165 170 175
Tyr Gly Phe Ser Leu Ser Ala Val Tyr Thr Cys Lys Arg Asp Pro Cys
              180 185 190
Pro His Gln Val Asp Cys Phe Leu Ser Arg Pro Thr Glu Lys Thr Ile
          195 200 205
Phe Ile Ile Phe Met Leu Val Val Ser Leu Val Ser Leu Ala Leu Asn
    210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                    245 250 255
Ala Lys Asp Cys Gly Ser Gln Lys Tyr Ala Tyr Phe Asn Gly Cys Ser
              260 265 270
Ser Pro Thr Ala Pro Leu Ser Pro Met Ser Pro Pro Gly Tyr Lys Leu
          275 280 285
Val Thr Gly Asp Arg Asn Asn Ser Ser Cys Arg Asn Tyr Asn Lys Gln
      290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                    325 330 335
Phe Pro Asp Asp Asn Gln Asn Ser Lys Lys Leu Ala Ala Gly His Glu
              340 345 350
Leu Gln Pro Leu Ala Ile Val Asp Gln Arg Pro Ser Ser Arg Ala Ser
        355 360 365
Ser Arg Ala Ser Ser Arg Pro Arg Pro Asp Asp Leu Glu Ile
      370 375 380
```

In some embodiments, connexin 43 (Cx43) and other connexin peptide modulators useful in carrying out methods of the invention include peptides such as Peptide5, i.e., VDCFLSRPTEKT (SEQ ID NO:107), as Gap19, i.e., KQIEKKFK (SEQ ID NO:108), Gap26, i.e., VCYDKSFPISHVR (SEQ ID NO:102), Gap27, a peptide called alpha connexin carboxyl terminus 1 (αCT1), and more, each targeting different binding sites with varying specificity and size. The connexin mimetic peptide Gap27, targeted to the SRPTEKTIFFI sequence (amino acids 204-214) on the second extracellular loop of Cx43 (SEQ ID NO:109), is a versatile inhibitor of connexin-mediated communication. In some embodiments the connexin 43 modulator may comprise or consist essentially of, for example, a peptide or peptidomimetic comprising or consisting essentially of SRPTEKTIF (SEQ ID NO:110).

In addition to Peptide5, Gap19, Gap 26, and Gap 27, another peptidomimetic particularly useful in the compositions, doses and dosing methods and schedules, kits and articles of manufacture disclosed herein is the fusion peptide designated XG19, i.e., lclrpvGGKQIEIKKFK, wherein lower case denotes the D-isomer [SEQ ID NO:111]). The XG19 peptidomimetic and its connexin modulation actions are described in U.S. Pat. No. 11,466,069, incorporated in its entirety herein by reference, as noted above.

In some embodiments, the invention provides compositions for use in the methods of the invention for treating a subject or a patient disease, or administration to a subject (e.g., in a laboratory) comprising a construct comprising (a) a targeting carrier peptide derived from the X-protein of the Hepatitis B virus and (b) a peptide capable of interacting with an intracellular domain of a connexin. In some embodiments, the targeting carrier peptide derived from the X-protein of the Hepatitis B virus comprises an amino acid sequence selected from the group consisting of all targeting carrier peptide described in U.S. Pat. No. 11,466,069. In some embodiments, the peptide capable of interacting with an intracellular domain interacts with the intracellular domain of one or more of connexin Cx26, Cx30, Cx30.3, Cx31.1, Cx32, Cx36, Cx37, Cx40, Cx43, Cx45, Cx50 and Cx58, including those described herein. In some embodiments, the peptide capable of interacting with an intracellular domain interacts with the intracellular domain of connexin 43. In some embodiments, the peptide capable of interacting with the intracellular domain of connexin 43 includes any of the connexin 43 intracellular interacting peptides described in U.S. Pat. No. 11,466,069, as well as those peptides described herein that interact with the intracellular domain of connexin 43.

In some embodiments, the connexin 43 modulators which may be used in methods of the invention can comprise peptides having a sequence comprising or consisting essentially of, for example, one or more of the following sequences: “Peptide 1” ADCFLSRPTEKT (SEQ ID NO: 112), “Peptide 2” VACFLSRPTEKT (SEQ ID NO: 113), “Peptide 11” VDCFLSRPTAKT (SEQ ID NO: 114), “Peptide 12” VDCFLSRPTEAT (SEQ ID NO: 115), “Peptide 5” VDCFLSRPTEKT (SEQ ID NO: 107), “Mod1” CFLSRPTEKT (SEQ ID NO: 116) and “Mod2” LSRPTEKT (SEQ ID NO: 117). In some embodiments, the carboxy-terminus of an anti-connexin peptide or peptidomimetic modulator can be modified. In some embodiments, the carboxy-terminus modification can comprise n-alkyl chains which can optionally be further linked to hydrogen or other moieties. In some embodiments, the connexin 43 peptides can include or exclude any of the peptides listed above or disclosed herein.

In some embodiments, the peptide or peptidomimetic comprises or consists essentially of from 7 to 40 amino acids of a connexin including, e.g., SEQ ID NO: 101 (SRPTEKT), SEQ ID NO: 107 (VDCFLSRPTEKT) and does not comprise a connexin C-terminal peptide.

Anti-connexin agents include peptides having an amino acid sequence that comprises about 5 to 20 contiguous amino acids of a connexin protein such as connexin 43 (SEQ ID NO:100), peptides having an amino acid sequence that comprises about 8 to 15 contiguous amino acids of connexin 43, or peptides having an amino acid sequence that comprises about 11 to 13 contiguous amino acids of connexin 43. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of connexin 43. Other anti-connexin 43 modulators comprise the extracellular domains of connexin 43, for example, peptide or peptidomimetic comprising or consisting essentially of SRPTEKT (SEQ ID NO: 101) or VDCFLSRPTEKT (SEQ ID NO: 107).

In other anti-connexin compounds, mimetic peptides are based on the extracellular domains of connexin 43 corresponding to the amino acids at positions 37-76 and 178-208 of connexin 43 protein sequence. Thus, certain anti-connexin peptides useful in methods of the invention have an amino acid sequence comprising or consisting essentially of about 7 to about 40 of the amino acids corresponding to the regions at positions 37-76 and 178-208 of the connexin 43 protein sequence. The peptides need not have an amino acid sequence identical to those portions of the connexin 43 protein sequence, and conservative amino acid changes may be made such that the peptides retain binding activity or functional activity in the assays described herein and otherwise known in the art. In other embodiments, mimetic peptides are based on peptide target regions within the connexin protein other than the extracellular domains (e.g. portions of the connexin 43 protein sequence not corresponding to positions 37-76 and 178-208).

In addition to therapeutically effective modified or unmodified peptide or peptidomimetics comprising or consisting essentially of a portion of an extracellular or transmembrane domain or C-terminal domain of a connexin 43, for example, other embodiments include modified or unmodified peptides or peptidomimetics comprising or consisting essentially of a portion of, for example, an extracellular or transmembrane domain of one or more other target connexins may be used in methods of the invention described herein. Other embodiments include modified or unmodified peptides or peptidomimetics comprising or consisting essentially of, for example, a portion of an extracellular or transmembrane domain of one or more other connexins found in blood vessels (e.g. endothelium, etc.), cells of a target organ (e.g., kidney, liver, eye, brain, skin, etc.).

In some embodiments, the anti-connexin peptidomimetic useful in a method of the invention is a connexin 45 peptidomimetic modulator comprising portions of the connexin 45 protein that antagonize or inhibit or block connexin-connexin interactions. In some embodiments the connexin 45 modulator may comprise, for example, a peptide or peptidomimetic comprising or consisting essentially of a portion of the E2 or C terminal domain of connexin 45, for example, comprising SRPTEKT (SEQ ID NO: 101). The peptide or peptidomimetic may also comprise, for example DCFISRPTEKT (SEQ ID NO: 118). Exemplary peptide sequences for connexin 45 peptides and peptidomimetic modulators useful in methods of the invention are also provided in Table 63 of U.S. Pat. No. 10,465,188.

In some embodiments, a connexin modulator comprises a peptide comprising an amino acid sequence corresponding to a portion of a transmembrane region of connexin 45 or a C-terminal region of connexin 45. In particular non-limiting embodiments, for example, the anti-connexin compound is a peptide having an amino acid sequence that comprises about 3 to about 30 contiguous amino acids of the known connexin 45 sequence, a peptide having an amino acid sequence that comprises about 5 to about 20 contiguous amino acids of the known connexin 45 sequence, a peptide having an amino acid sequence that comprises about 8 to about 15 contiguous amino acids of the known connexin 45 sequence, or a peptide having an amino acid sequence that comprises about 11, 12, or 13 contiguous amino acids of the known connexin 45 sequence. Other non-limiting embodiments include an anti-connexin compound that is a peptide having an amino acid sequence that comprises at least about 3, 4, 5, 6, 7, 8, 9, 10, 11, 12, 13, 14, 15, 20, 25, or 30 contiguous amino acids of a known connexin 45 sequence. In certain anti-connexin compounds provided herein, mimetic peptides are based on the extracellular domains of connexin 45 corresponding to the amino acids at positions 46-75 and 199-228 of a known connexin 45 sequence. Thus, certain peptide described herein have an amino acid sequence corresponding to the regions at positions 46-75 and 199-228 of a known connexin 45 sequence. The peptides need not have an amino acid sequence identical to those portions of known connexin 45 sequence, and conservative amino acid changes may be made such that the peptides retain binding activity or functional activity in the assays described herein and otherwise known in the art. In other embodiments, mimetic peptides are based on peptide target regions within the connexin protein other than the extracellular domains (e.g. portions of a known connexin 45 sequence not corresponding to positions 46-75 and 199-228). See WO2006/134494, disclosing various connexin sequences.

Some peptidomimetic connexin modulators useful in methods of the invention include VDCFLSRPTEKT (SEQ ID NO: 107) and SRPTEKTIFII (SEQ ID NO: 104), which bind, for example, to the Cx43 extracellular domain.

In some embodiments, the connexin 26 peptidomimetic connexin modulator useful in a method of the invention and dosed according to the one or more of the schedules described herein is Gap26. In other embodiments, the anti-connexin peptidomimetic for use in a method of the invention is a connexin32 peptidomimetic (e.g. INCTLQPGCNSV (SEQ ID NO: 103) or ³²Gap27, i.e., SRPTEKTIFII (SEQ ID NO:104) or a connexin50 peptidomimetic (e.g. TAT-Cx50L2, i.e., GGERAPLAADQGSVKKSSSSSKGTKK (SEQ ID NO:105) or TAT-Cx50CT, i.e., SRARSDDLTV (SEQ ID NO:106)).

In some embodiments, peptides and peptidomimetics include peptides and peptidomimetics useful for the inhibition of gap junction channels and hemichannels corresponding to specific sequences within extracellular loops E1 and E2 involving the conserved QPG and SHVR motifs of E1 (Gap26 peptide) and the SRPTEK motif in E2 (Gap27 peptide) as well as the cytoplasmic loop (Gap19 peptide). Useful peptidomimetic connexin modulators are described in U.S. Pat. No. 9,248,141 (“Methods of treatment by administering anti-connexin peptides and mimetics”). Other useful peptide connexin modulators are described in U.S. Pat. No. 11,466,069 (“Methods of treatment and novel constructs”), including XG19 and other constructs comprising a targeting carrier peptide derived from the X-protein of the Hepatitis B virus and a peptide capable of interacting with an intracellular domain of a connexin (e.g. connexin 43).

Other connexin peptide modulators useful in methods of the invention are provided in Table 64 of U.S. Pat. No. 10,465,188. Useful peptide modulators of connexin 43 (Cx43) and other connexins that may be dosed in embodiments of the invention are also referenced in Caufriez, A., et al., Expert Opinion on Drug Discovery 15(10):1213-1222 (2020), and in King, D R, et al., Mechanisms of Connexin Regulating Peptides Int. J. Mol. Sci. 22:10186 (September 2021) (see, e.g., FIG. 1 (“Schematic of the Cx43 protein in the plasma membrane with colored lines indicating the positions of described peptides targeting EL, IL and CT regions”) and Table 1 (“Connexin regulating peptides”).

In some embodiments the peptides may also be used as promoieties. See, e.g. Vig, B S, et al., Amino acids as promoieties in drug design and Development. Advanced Drug Delivery Reviews 65(10): p 1370-1385 (2013); Dhokchawle, B, et al., Promoieties Used In Prodrug Design: A Review. Indian Journal of Pharmaceutical Education 48(2):35-40 (2013).

Connexin hemichannel-blocking or modulating peptides or peptidomimetics may be unmodified, or modified as desired (e.g. to increase stability, to further stabilize peptide conformation, increase bioactivity, increase cell permeability, etc.). See e.g. DeGruyter, J N, et al., Residue-Specific Peptide Modification: A Chemist's Guide. Biochemistry 56, 30, 3863-3873 (2017); Boto, A, et al. Site-selective modification of peptide backbones. Org. Chem. Front. 8:6720-6759 (2021) (review article). Thus, for example, peptidomimetics used in methods of the invention may contain one or more modified amino acids, amino acid analogs, or may be otherwise modified to improve bioavailability or to increase penetration across the cell membrane.

Other peptide sequences known to inhibit the inter-connexin binding that can regulate connexin activity are the cytoplasmic loop of connexin 43 (amino acids 119-144) L2 peptide and subparts of the L2 peptide of connexin 43. In some embodiments, these peptides may include or exclude, for example, the nine amino acid sequence of Gap19, KQIEIKKFK (SEQ ID NO: 108); the native Gap19 sequence, DGVNVEMHLKQIEIKKFKYGIEEHGK (SEQ ID NO: 119); the His144-Glu L2 derivative of Gap19, as reported by Shibayama (Shibayama, J. et al., Biophys. J. 91, 405404063, 2006), DGVNVEMHLKQIEIKKFKYGIEEQGK (SEQ ID NO: 120); the TAT-Gap19 sequence, YGRKKRRQRRRKQIEIKKFK (SEQ ID NO: 121); the SH3 binding domain, CSSPTAPLSPMSPPGYK (SEQ ID NO: 122), or subpart thereof PTAPLSPMSPP (SEQ ID NO: 123); the C-terminal sequence of the CT9 or CT10 peptide, with or without a TAT leader sequence to increase cell penetration, for example, RPRDDEI (CT9; SEQ ID NO: 124), SRPRDDLEI (CT10; SEQ ID NO: 125), YGRKKRRQRRRSRPRDDEI (TAT-CT9; SEQ ID NO: 126), or YGRKKRRQRRRRPRDDEI (TAT-CT10; SEQ ID NO: 127). Other peptidomimetic sequences that can be included or excluded in the compositions, methods, kits or articles of manufacture disclosed herein are those reported by Dhein (Dhein, S., Naunyn-Schmiedeberg's Arch. Pharm., 350: 174-184, 1994); the AAP10 peptide, H₂N-Gly-Ala-Gly-4Hyp-Pro-Tyr-CONH₂ (SEQ ID NO: 128), and the ZP123 peptide (rotigapeptide), Ac-D-Tyr-Pro-D-4Hyp-Gly-D-Ala-Gly-NH₂ (SEQ ID NO: 129), (Dhein, S., et al. Cell Commun. Adhes. 10, 371-378, 2013). Rotigapeptide is comprised of the D-form of the peptides for enhanced efficacy over the native L-form of the peptide.

In some embodiments, the therapeutically effective modified or unmodified peptide or peptidomimetic comprises a portion of the E1 extracellular domain of a connexin, such as the connexin 43 E1 (ESAWGDEQSAFRCNTQQPGCENVCYDKSFPISHVR; SEQ ID NO:130) or the connexin 45 E1 (GESIYYDEQSKFVCNTEQPGCENVCYDAFAPLSHVR; SEQ ID NO:131). In some embodiments, the therapeutically effective modified or unmodified peptide or peptidomimetic comprises a portion of the E2 extracellular domain of a connexin, such as the connexin 43 E2 (LLIQWYIYGFSLSAVYTCKRDPCPHQVDCFLSRPTEKT; SEQ ID NO:132) or the connexin 45 E2 (LIGQYFLYGFQVHPFYVCSRLPCHPKIDCFISRPTEKT; SEQ ID NO:133).

In certain embodiments, the connexin 43 modulator peptides of the present invention can be linked at the amino or carboxy terminus to a cellular internalization transporter. The cellular internalization transporter linked to connexin 43 modulator peptides of the present invention may be any internalization sequence known or newly discovered in the art, or conservative variants thereof. Non-limiting examples of cellular internalization transporters and sequences include antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (BisGuanidinium-Tren-Cholesterol). Cellular internalization transporters are useful for peptidomimetics such as Gap19 and the aCT peptides. The sequences of exemplary cellular internalization peptides are known in the art. See, e.g., Table 65 in U.S. Pat. Nos. 10,465,188; 11,466,069.

In other embodiments, the peptide or peptidomimetic may be modified to increase transfection uptake by conjugating the peptide to a hydrophobic compound, in some embodiments, through a linker moiety. The hydrophobic compound may be, for example, one or more n-alkyl groups, which may be, for example, C6-C14 alkyl groups. In some embodiments, the peptides may be conjugated at the N terminus to one or two dodecyl (C12) groups as described in Chen, Y S et al., J. Pharm. Sci., 102: 2322-2331 (2013). In one embodiment, the peptide sequence CFLSRPTEKT (SEQ ID NO:116) or VDCFLSRPTEKT (SEQ ID NO:107) can be conjugated to two dodecyl groups to create a modified peptide which can modulate connexin 43, “C12-C12-Cxn43 MP.” See SEQ ID NO: 326 in U.S. Pat. No. 10,465,188.

In some embodiments, the therapeutically effective modified or unmodified peptide or peptidomimetic comprises a portion of the C-terminal domain of a connexin, such as connexin 43 or connexin 45, preferably connexin 43. Some embodiments of anti-connexin 43 modulators useful in methods of the invention comprise the C-terminus region of connexin 43, or modified versions thereof. See, e.g., O'Quinn, M P, et al., A Peptide Mimetic of the Connexin43 Carboxyl-Terminus Reduces Gap Junction Remodeling and Induced Arrhythmia Following Ventricular Injury. Circ Res. 108(6): 704-715 (March 2011). C-terminus connexin peptidomimetic modulators, including αCT1 (alpha connexin carboxy terminus 1) peptides (also referred to as aCT1 or ACT1 peptides in publications), are described, for example, in Montgomery et al., Connexin 43-Based Therapeutics for Dermal Wound Healing Int. J. Mol. Sci. 2018, 19, 1778, and U.S. Pat. No. 8,815,556 (“Compositions and methods for tissue engineering, tissue regeneration and wound healing”). See also WO2006/069181. Preferred connexin carboxy-terminal polypeptides are connexin 43 carboxy-terminal polypeptides. Such compounds are described in US Patent Publication No. 20070042964 (“Compositions and methods for modulating connexin hemichannels”). If a connexin peptide or peptidomimetic modulator comprises a portion of an intracellular domain of a connexin (e.g. an aCT peptide, such as CT9, CT10, αCT1, etc.), the peptide may be conjugated to a cell internalization transporter including those noted or referenced herein. In some embodiments, a connexin peptidomimetic modulator useful in methods of then invention may block zona occludens (ZO-1) binding to connexin 43 and modulate connexin gap junctions and hemichannels to advantage. See FIG. 2 in Caufriez, A, et al. Peptide-based targeting of connexins and pannexins for therapeutic purposes. Expert Opinion on Drug Discovery 15(10):1213-1222 (2020).

In some embodiments, the connexin modulator may be a gap junction closing compound and/or hemichannel closing compound. In some embodiments, the gap junction closing compounds and hemichannel closing compounds are connexin 43 gap junction closing compounds and connexin 43 hemichannel closing compounds (e.g. a Cx43 C-terminus peptidomimetic).

Various useful peptidomimetic peptides mimic sequences of connexin extracellular regions. The first extracellular loops of Cx37, Cx40 as well Cx43 are mimicked by peptides under the Gap26 code. Gap27 and Peptide5 mimic regions of the second extracellular loop. Gap27 targets Cx32, Cx40 and Cx43, while Peptide5 is used for Cx43 inhibition. JM2, ASH3, CT9, CT10, aCT mimic the C-terminal tail of Cx43 and Gap24 reproduces a sequence of the cytoplasmic loop of Cx32. Both L2 and Gap19 also mimic the cytoplasmic loop but of Cx43.

The extracellular loops of connexin hemichannels are also good targets for peptidomimetic connexin modulators useful in the invention due to their accessibility in contrast to their full channel counterparts. Nevertheless, peptides containing the conserved motives QPG and SHVR of the first extracellular loop and the SRPTEK motif of the second extracellular loop interfere with the formation of gap junctions. This led to the development of peptide mimetics ⁴³Gap26, ^{37,40}Gap26, ³²Gap27, ⁴⁰Gap27, ⁴³Gap27 and ⁴³Peptide5 (note that the superscript in the nomenclature of these peptide analogues refers to Cx subtypes they can target).

The conserved SHVR motif of the first extracellular loop is incorporated in the sequence of useful Gap26 peptide mimetics. The two slightly different sequences are both categorized under the Gap26 code, one targeting Cx37 and Cx40, and the other targeting only Cx43. Cells treated with either of these Gap26 peptides showed Cx hemichannel inhibition within minutes.

There are three Gap27 peptides, each targeting different Cx types, namely Cx32, Cx40 and Cx43 that are useful in compositions and methods of the invention. These peptides mimic the conserved SRPTEK motif of the second extracellular loop, but have the same time-dependent effect on gap junction activity as Gap26. Like Gap27, Peptide5 contains the SRPTEK motif. However, the mimicked sequence of Peptide5 is shifted in the direction of the N-terminal tail in comparison to that of Gap27. Peptide5 can inhibit Cx43 hemichannels at a concentration of 5-10 M, but incubation at higher concentration (100 M or higher) can also leads to inhibition of gap junctions in some circumstances.

Other peptides useful in compositions and methods of the invention mimic sequences of connexin intracellular regions. The interaction between the cytoplasmic loop and C-terminal tail mediates the gating mechanism of connexin hemichannels and gap junctions. Gap junctions are in an open state when there is no interaction between the C-terminal tail and cytoplasmic loop, while such interaction is critical for connexin hemichannel opening. The CT10 peptidomimetic reproduces the last 10 amino acids of the C-terminal tail of Cx43. Inhibition of connexin 43-(Cx43)-mediated ATP release by a peptide mimetic, called TAT-L2, pinpointed its mimicked L2 region (amino acid 119 to 144) as an essential sequence of the cytoplasmic loop in the interaction with the Cx43 C-terminal tail. To date, 2 peptides mimicking the L2 region are available, namely ⁴³Gap19 and ³²Gap24. ⁴³Gap19 inhibits Cx43 hemichannel currents by binding to the C-terminal tail, thereby inhibiting the cytoplasmic loop/C-terminal tail interaction. ⁴³Gap19 has the advantage of being a selective inhibitor, as it does not affect gap junction or Panx1 channel activity. ³²Gap24 is a peptide that mimics a 13 amino acid long stretch of the L2 region of Cx32 and can also be used in methods of the invention. Cx32 is one of the ten human corneal epithelial connexin that can be usefully targeted as described herein. In vitro studies showed that Cx32 hemichannel-mediated ATP release is inhibited by ³²Gap24 at concentrations of 17 M without affecting gap junctions.

Peptides targeting intracellular regions of connexin proteins need to access the intracellular environment. Cell-penetrating peptides for this purpose are described herein (e.g. the TAT-peptide, an oligoarginine tag and the Xentry peptide), and can be anchored to connexin-derived peptide sequences in order to enhance the uptake into the cell via endocytosis. 43Gap19 can enter the cell on its own due to the KKFK cell-translocation motif of the L2 region. Nonetheless, comparison of the IC₅₀ (half maximal inhibitory concentration) of ⁴³Gap19 itself (47 μM) and TAT-⁴³Gap19 (7 μM) for the inhibition of ATP release in glioma cells showed that the entry of ⁴³Gap19 into the cell can be improved by linking to a TAT-tag. All 5 available peptides mimicking the C-terminal tail are derived from Cx43.

The αCT1 peptidomimetic mimics the last 9 amino acids of Cx43 and is linked to an antennapedia sequence that facilitates cellular internalization of the peptide mimetic. The interaction between Cx43 and the PDZ-domain of ZO-1, a region that is suggested to be involved in the regulation of Cx trafficking and gap junction assembly, is disrupted by αCT1. This interference reportedly leads to increased gap junction plaque formation and decreased Cx hemichannel activity.

If a peptide or peptidomimetic modulator comprises a portion of an intracellular domain of a connexin, the peptide may, in some embodiments, be conjugated to a cell internalization transporter and may, in some instances, block zona occludens (ZO-1) binding to connexin 43.

In some embodiments of this invention, the connexin modulator is a peptide or peptidomimetic shown in Table II below (E2 and T2 refer to the location of a peptide in, for example, the second extracellular domain or the second transmembrane domain).

TABLE II

| SEQ ID NO: | Identifier | Sequence |
|---|---|---|
| SEQ ID NO: 140 | CXT 2 | PSSRASSRASSRPRPDDLEI |
| SEQ ID NO: 141 | CXT 1 | RPRPDDLEI |
| SEQ ID NO: 142 | CXT 3 | RPRPDDLEV |
| SEQ ID NO: 143 | CXT 4 | RPRPDDVPV |
| SEQ ID NO: 144 | CXT 5 | KARSDDLSV |
| SEQ ID NO: 145 | hCx40 | QKPEVPNGVSPGHRLPHGYHSDKRRLSKASSKARSD |
| | | DLSV |
| SEQ ID NO: 146 | Antp/CXT 2 | RQPKIWFPNRRKPWKKPSSRASSRASSRPRPDDLEI |
| SEQ ID NO: 147 | Antp/CXT 2 | RQPKIWFPNRRKPWKKPSSRASSRASSRPRPDDLEI |
| SEQ ID NO: 148 | Antp/CXT 1 | RQPKIWFPNRRKPWKKRPRPDDLEI |
| SEQ ID NO: 149 | Antp/CXT 3 | RQPKIWFPNRRKPWKKRPRPDDLEV |
| SEQ ID NO: 150 | Antp/CXT 4 | RQPKIWFPNRRKPWKKRPRPDDVPV |
| SEQ ID NO: 151 | Antp/CXT 5 | RQPKIWFPNRRKPWKKKARSDDLSV |
| SEQ ID NO: 152 | conservative | RPKPDDLDI |
| | Cx43 variant | |
| SEQ ID NO: 153 | HIV-Tat/CXT | GRKKRRQRPPQRPRPDDLEI |
| | 1 | |
| SEQ ID NO: 154 | Penetratin/CXT | RQIKIWFQNRRMKWKKRPRPDDLEI |
| | 1 | |
| SEQ ID NO: 155 | Antp-3A/CXT | RQIAIWFQNRRMKWAARPRPDDLEI |
| | 1 | |
| SEQ ID NO: 156 | Tat/CXT 1 | RKKRRQRRRRPRPDDLEI |
| SEQ ID NO: 157 | Buforin II/Vnrs | TRSSRAGLQFPVGRVHRLLRKRPRPDDLEI |
| | 1 | |
| SEQ ID NO: 158 | Transportan/ | GWTLNSAGYLLGKINKALAALAKKILRPRPDDLEI |
| | CXT 1 | |
| SEQ ID NO: 159 | MAP/CXT 1 | KLALKLALKALKAALKLARPRPDDLEI |
| SEQ ID NO: 160 | K-FGF/CXT 1 | AAVALLPAVLLALLAPRPRPDDLEI |
| SEQ ID NO: 161 | Ku70/CXT 1 | VPMLKPMLKERPRPDDLEI |
| SEQ ID NO: 162 | Prion/CXT 1 | MANLGYWLLALFVTMWTDVGLCKKRPKPRPRPDDL |
| | | EI |
| SEQ ID NO: 163 | pVEC/CXT 1 | LLIILRRRIRKQAHAHSKRPRPDDLEI |
| SEQ ID NO: 164 | Pep-1/CXT 1 | KETWWETWWTEWSQPKKKRKVRPRPDDLEI |
| SEQ ID NO: 165 | SynB1/CXT 1 | RGGRLSYSRRRFSTSTGRRPRPDDLEI |
| SEQ ID NO: 166 | Pep-7/CXT 1 | SDLWEMMMVSLACQYRPRPDDLEI |
| SEQ ID NO: 167 | HN-1/CXT 1 | TSPLNIHNGQKLRPRPDDLEI |
| SEQ ID NO: 107 | Peptide 5 | VDCFLSRPTEKT |
| SEQ ID NO: 104 | ^{37,43}Gap27 or | SRPTEKTIFII |
| | ³²Gap27 | |
| SEQ ID NO: 102 | Gap26 | VCYDKSFPISHVR |
| SEQ ID NO: 116 | SEQ-Mod1 | CFLSRPTEKT |
| SEQ ID NO: 117 | SEQ-Mod2 | LSRPTEKT |
| SEQ ID NO: 101 | | SRPTEKT |
| SEQ ID NO: 174 | SEQ-Mod4 | VDCFLSRPTE |
| SEQ ID NO: 175 | SEQ-Mod5 | VDCFLSRP |
| SEQ ID NO: 176 | SEQ-Mod6 | VDCFLS |
| SEQ ID NO: 177 | HIV-Tat/SEQ- | GRKKRRQRPPQVDCFLSRPTEKT |
| | pept5 | |
| SEQ ID NO: 178 | Penetratin/ | RQIKIWFQNRRMKWKKVDCFLSRPTEKT |
| | SEQ-pept5 | |
| SEQ ID NO: 179 | Antp-3A/SEQ- | RQIAIWFQNRRMKWAAVDCFLSRPTEKT |
| | pept5 | |
| SEQ ID NO: 180 | Tat/SEQ-pept5 | RKKRRQRRRVDCFLSRPTEKT |
| SEQ ID NO: 181 | Buforin II/ | TRSSRAGLQFPVGRVHRLLRKVDCFLSRPTEKT |
| | SEQ-pept5 | |
| SEQ ID NO: 182 | Transportan/ | GWTLNSAGYLLGKINKALAALAKKILVDCFLSR |
| | SEQ-pept5 | PTEKT |
| SEQ ID NO: 183 | MAP/SEQ- | KLALKLALKALKAALKLAVDCFLSRPTEKT |
| | pept5 | |
| SEQ ID NO: 184 | K-FGF/SEQ- | AAVALLPAVLLALLAPVDCFLSRPTEKT |
| | pept5 | |
| SEQ ID NO: 185 | Ku70/SEQ- | VPMLKPMLKEVDCFLSRPTEKT |
| | pept5 | |
| SEQ ID NO: 186 | Prion/SEQ- | MANLGYWLLALFVTMWTDVGLCKKRPKPVDCFLSR |
| | pept5 | PTEKT |
| SEQ ID NO: 187 | pVEC/SEQ- | LLIILRRRIRKQAHAHSKVDCFLSRPTEKT |
| | pept5 | |
| SEQ ID NO: 188 | Pep-1/SEQ- | KETWWETWWTEWSQPKKKRKVVDCFLSRPTEKT |
| | pept5 | |
| SEQ ID NO: 189 | SynB1/SEQ- | RGGRLSYSRRRFSTSTGRVDCFLSRPTEKT |
| | pept5 | |
| SEQ ID NO: 190 | Pep-7/SEQ- | SDLWEMMMVSLACQYVDCFLSRPTEKT |
| | pept5 | |
| SEQ ID NO: 191 | HN-1/SEQ- | TSPLNIHNGQKLVDCFLSRPTEKT |
| | pept5 | |
| SEQ ID NO: 192 | SEQ M3E2 | FEVAFLLIQWI |
| SEQ ID NO: 193 | SEQ E2a | LLIQWYIGFSL |
| SEQ ID NO: 194 | SEQ E2b | SLSAVYTCKRDPCPHQ |
| SEQ ID NO: 196 | SEQ MIE1 | LGTAVESAWGDEQ |
| SEQ ID NO: 197 | SEQ Ela | QSAFRCNTQQPG |
| SEQ ID NO: 198 | SEQ E1b | QQPGCENVCYDK |
| SEQ ID NO: 199 | SEQ E2d | KRDPCHQVDCFLSRPTEK |
| SEQ ID NO: 112 | Peptide 1 | ADCFLSRPTEKT |
| SEQ ID NO: 113 | Peptide 2 | VACFLSRPTEKT |
| SEQ ID NO: 114 | Peptide 11 | VDCFLSRPTAKT |
| SEQ ID NO: 115 | Peptide 12 | VDCFLSRPTEAT |
| SEQ ID NO: 108 | Gap 19-subpart | KQIEIKKFK |
| SEQ ID NO: 119 | Gap 19-full | DGVNVEMHLKQIEIKKFKYGIEEHGK |
| SEQ ID NO: 120 | Gap 19-deriv | DGVNVEMHLKQIEIKKFKYGIEEQGK |
| SEQ ID NO: 121 | TAT-Gap19 | YGRKKRRQRRRKQIEIKKFK |
| SEQ ID NO: 122 | SH3-full | CSSPTAPLSPMSPPGYK |
| SEQ ID NO: 123 | SH3-subpart | PTAPLSPMSPP |
| SEQ ID NO: 128 | AAP10 | H2N-Gly-Ala-Gly-4Hyp-ProTyr-CONH2 |
| SEQ ID NO: 129 | ZP123 | Ac-D-Tyr-Pro-D-4Hyp-Gly-D-Ala-Gly-NH2 |
| SEQ ID NO: 168 | plsl/SEQ-pept5 | RVIRVWFQNKRCKDKKVDCFLSRPTEKT |
| SEQ ID NO: 169 | MGB Peptide | GALFLGFLGAAGSTMGAWSQPKKKRKVVDCFLSRPT |
| | P-beta/SEQ- | EKT |
| | pept5 | |
| SEQ ID NO: 170 | MGB Peptide | GALFLAFLAAALSLMGLWSQPKKKRRVVDCFLSRPT |
| | P-alpha/SEQ- | EKT |
| | pept5 | |
| SEQ ID NO: 171 | huCx26 | MYVFYVMYDGFSMQRLVKCNAWPCPNTVDCFVSRP |
| | | TEKT |
| SEQ ID NO: 172 | huCx30 | MYVFYFLYNGYHLPWVLKCGIDPCPNLVDCFISRPTE |
| | | KT |
| SEQ ID NO: 173 | huCx30.3 | LYIFHRLYKDYDMPRVVACSVEPCPHTVDCYISRPTE |
| | | KK |
| SEQ ID NO: 195 | huCx31 | LYLLHTLWHGFNMPRLVQCANVAPCPNIVDCYIARP |
| | | TEKK |
| SEQ ID NO: 200 | huCx31.1 | LYVFHSFYPKYILPPVVKCHADPCPNIVDCFISKPSEK |
| | | N |
| SEQ ID NO: 201 | huCx32 | MYVFYLLYPGYAMVRLVKCDVYPCPNTVDCFVSRP |
| | | TEKT |
| SEQ ID NO: 202 | huCx36 | LYGWTMEPVFVCQRAPCPYLVDCFVSRPTEKT |
| SEQ ID NO: 203 | huCx37 | LYGWTMEPVFVCQRAPCPYLVDCFVSRPTEKT |
| SEQ ID NO: 204 | huCx40.1 | GALHYFLFGFLAPKKFPCTRPPCTGVVDCYVSRPTEK |
| | | S |
| SEQ ID NO: 205 | huCx46 | IAGQYFLYGFELKPLYRCDRWPCPNTVDCFISRPTEK |
| | | T |
| SEQ ID NO: 206 | huCx46.6 | LVGQYLLYGFEVRPFFPCSRQPCPHVVDCFVSRPTEK |
| | | T |
| SEQ ID NO: 207 | huCx40 | IVGQYFIYGIFLTTLHVCRRSPCPHPVNCYVSRPTEKN |

In some embodiments the connexin 43 modulator may comprise, for example, a peptide or peptidomimetic comprising, for example SEQ ID NO: 101 (SRPTEKT). The peptide or peptidomimetic may also comprise, for example SEQ ID NO: 107 (VDCFLSRPTEKT). The peptide may contain one or more modified amino acids, amino acid analogs, or may be otherwise modified to improve bioavailability or to increase penetration across the cell membrane. For example, SEQ ID NO: 107 may be modified to obtain SEQ ID NOS: 177-191. In some embodiments, the peptide or peptidomimetic comprising, for example SEQ ID NO: 101 (SRPTEKT) or SEQ ID NO: 107 (VDCFLSRPTEKT) comprises from 7 to 40 amino acids or amino acid analogues and does not comprise a C-terminal peptide. In some embodiments the peptides may also be used as promoieties.

In some embodiments, the Connexin 45 modulators can be peptide or peptidomimetics comprising portions of the Connexin 45 protein that antagonize or inhibit or block connexin-connexin interactions. Exemplary peptide sequences for Connexin 45 peptides and peptidomimetic modulators are provided in Table III.

TABLE III
SEQUENCES OF CONNEXIN 45 MODULATOR
PEPTIDES OR PEPTIDOMIMETICS

| SEQ ID NO. | Sequence |
|---|---|
| SEQ ID NO: | LTAVGGESIYYDEQSKFVCNTEQPGC |
| 208 | ENVCYDAFAPLSHVRFWVFQ |
| SEQ ID NO: | LTAVGGESIYYDEQS |
| 209 | |
| SEQ ID NO: | DEQSKFVCNTEQP |
| 210 | |
| SEQ ID NO: | TEQPGCENVCYDA |
| 211 | |
| SEQ ID NO: | VCYDAFAPLSHVR |
| 212 | |
| SEQ ID NO: | APLSHVRFWVFQ |
| 213 | |
| SEQ ID NO: | FEVGFLIGQYF |
| 214 | |
| SEQ ID NO: | LIGQYFLYGFQV |
| 215 | |
| SEQ ID NO: | GFQVHPFYVCSRLP |
| 216 | |
| SEQ ID NO: | SRLPCHPKIDCF |
| 217 | |
| SEQ ID NO: | IDCFISRPTEKT |
| 218 | |
| SEQ ID NO: | SRPTEKTIFLL |
| 219 | |
| SEQ ID NO: | YVCSRLPCHP |
| 220 | |
| SEQ ID NO: | QVHPFYVCSRL |
| 221 | |
| SEQ ID NO: | FEVGFLIGQYFLY |
| 222 | |
| SEQ ID NO: | GQYFLYGFQVHP |
| 223 | |
| SEQ ID NO: | GFQVHPFYVCSR |
| 224 | |
| SEQ ID NO: | AVGGESIYYDEQ |
| 225 | |
| SEQ ID NO: | YDEQSKFVCNTE |
| 226 | |
| SEQ ID NO: | NTEQPGCENVCY |
| 227 | |
| SEQ ID NO: | CYDAFAPLSHVR |
| 228 | |
| SEQ ID NO: | FAPLSHVRFWVF |
| 229 | |
| SEQ ID NO: | LIGQY |
| 230 | |
| SEQ ID NO: | QVHPF |
| 231 | |
| SEQ ID NO: | YVCSR |
| 232 | |
| SEQ ID NO: | SRLPC |
| 233 | |
| SEQ ID NO: | LPCHP |
| 234 | |
| SEQ ID NO: | GESIY |
| 235 | |
| SEQ ID NO: | YDEQSK |
| 236 | |
| SEQ ID NO: | SKFVCN |
| 237 | |
| SEQ ID NO: | TEQPGCEN |
| 238 | |
| SEQ ID NO: | VCYDAFAP |
| 239 | |
| SEQ ID NO: | LSHVRFWVFQ |
| 240 | |
| SEQ ID NO: | LIQYFLYGFQVHPF |
| 241 | |
| SEQ ID NO: | VHPFYCSRLPCHP |
| 242 | |
| SEQ ID NO: | VGGESIYYDEQSKFVCNTEQPG |
| 243 | |
| SEQ ID NO: | TEQPGCENVCYDAFAPLSHVRF |
| 244 | |
| SEQ ID NO: | AFAPLSHVRFWVFQ |
| 245 | |
| SEQ ID NO: | IDCFISRPTEKTIFLL |
| 246 | |
| SEQ ID NO: | DCFISRPTEKT |
| 118 | |
| SEQ ID NO: | SRPTEKT |
| 101 | |

In some embodiments the connexin 45 modulator may comprise, for example, a peptide or peptidomimetic comprising, a portion of the E2 or C terminal domain of connexin 45, for example, comprising SEQ ID NO: 101 (SRPTEKT). The peptide or peptidomimetic may also comprise, for example SEQ ID NO: 118 (DCFISRPTEKT). In some embodiments the peptides may only be 3 amino acids in length, including SRL, PCH, LCP, CHP, IYY, SKF, QPC, VCY, APL, HVR, or longer.

When specific proteins are referred to herein, derivatives, variants, and fragments are contemplated and included. Protein derivatives and variants are well understood to those of skill in the art and can involve insertional, substitutional or deletional amino acid sequence variants known in the art.

The gap junction modulators and anti-connexin hemichannel blocking peptides or peptidomimetics are made chemically, synthetically, or otherwise manufactured.

### Antisense Connexin Hemichannel Modulators

A gap junction and/or connexin polynucleotide or oligonucleotide may be selected, for example, from modified or unmodified connexin polynucleotides or oligonucleotides (e.g. modified or unmodified connexin 43 antisense polynucleotides or oligonucleotides). In some embodiments, the modified connexin antisense polynucleotides, or oligonucleotides or polynucleotides comprise mixtures of modified and unmodified nucleotides. In some embodiments, the connexin 43 antisense compound used in the methods herein is an antisense oligonucleotide comprising naturally occurring nucleobases and an unmodified internucleoside linkage.

In some embodiments, the connexin 43 antisense compound is targeted to at least about 8 nucleobases of a nucleic acid molecule encoding a connexin having a nucleobase sequence selected from SEQ ID NO:17. The polynucleotides and oligonucleotides, for example, connexin 43 antisense compounds, may have from about 8 to about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or about 80 nucleotides of SEQ ID NO:17, or a sequence complementary thereto, and/or the antisense polynucleotide or oligonucleotide may contain any range of lengths between any two of the recited lengths. The polynucleotides of this invention include synthesized polynucleotides having a length of less than 80 nucleotides, e.g., from 12-18 to about 50-80 nucleotides, preferably about 30 nucleotides or less, e.g., from 12 to about 30 nucleotides, and more preferably from about 15 to about 30 nucleotides. In one example, the polynucleotide has 30 nucleotides. The methods of this invention features, in some embodiments, the use of connexin 43 antisense compounds up to 40 nucleotides in length, for example, 15 to 40 nucleotides in length, comprising or consisting essentially of a nucleotide sequence selected from SEQ ID NO:1-17. The methods of this invention features, in some embodiments, the use of connexin 43 antisense compounds up to 40 nucleotides in length, for example, 15 to 40 nucleotides in length, comprising a nucleotide sequence selected from SEQ ID NO:4-17.

```
Human Cx 43, 1
LOCUS NM_000165 3088 bp mRNA linear PRI OCT. 26, 2004
DEFINITION Homo sapiens gap junction protein, alpha 1,
43 kDa (connexin 43)(GJA1), mRNA.
(SEQ ID NO: 17)
      1 acaaaaaagc ttttacgagg tatcagcact tttctttcat tagggggaag gcgtgaggaa
    61 agtaccaaac agcagcggag ttttaaactt taaatagaca ggtctgagtg cctgaacttg
  121 ccttttcatt ttacttcatc ctccaaggag ttcaatcact tggcgtgact tcactacttt
  181 taagcaaaag agtggtgccc aggcaacatg ggtgactgga gcgccttagg caaactcctt
  241 gacaaggttc aagcctactc aactgctgga gggaaggtgt ggctgtcagt acttttcatt
  301 ttccgaatcc tgctgctggg gacagcggtt gagtcagcct ggggagatga gcagtctgcc
  361 tttcgttgta acactcagca acctggttgt gaaaatgtct gctatgacaa gtctttccca
  421 atctctcatg tgcgcttctg ggtcctgcag atcatatttg tgtctgtacc cacactcttg
  481 tacctggctc atgtgttcta tgtgatgcga aaggaagaga aactgaacaa gaaagaggaa
  541 gaactcaagg ttgcccaaac tgatggtgtc aatgtggaca tgcacttgaa gcagattgag
  601 ataaagaagt tcaagtacgg tattgaagag catggtaagg tgaaaatgcg aggggggttg
  661 ctgcgaacct acatcatcag tatcctcttc aagtctatct ttgaggtggc cttcttgctg
  721 atccagtggt acatctatgg attcagcttg agtgctgttt acacttgcaa aagagatccc
  781 tgcccacatc aggtggactg tttcctctct cgccccacgg agaaaaccat cttcatcatc
  841 ttcatgctgg tggtgtcctt ggtgtccctg gccttgaata tcattgaact cttctatgtt
  901 ttcttcaagg gcgttaagga tcgggttaag ggaaagagcg acccttacca tgcgaccagt
  961 ggtgcgctga gccctgccaa agactgtggg tctcaaaaat atgcttattt caatggctgc
1021 tcctcaccaa ccgctcccct ctcgcctatg tctcctcctg ggtacaagct ggttactggc
1081 gacagaaaca attcttcttg ccgcaattac aacaagcaag caagtgagca aaactgggct
1141 aattacagtg cagaacaaaa tcgaatgggg caggcgggaa gcaccatctc taactcccat
1201 gcacagcctt ttgatttccc cgatgataac cagaattcta aaaaactagc tgctggacat
1261 gaattacagc cactagccat tgtggaccag cgaccttcaa gcagagccag cagtcgtgcc
1321 agcagcagac ctcggcctga tgacctggag atctagatac aggcttgaaa gcatcaagat
1381 tccactcaat tgtggagaag aaaaaaggtg ctgtagaaag tgcaccaggt gttaattttg
1441 atccggtgga ggtggtactc aacagcctta ttcatgaggc ttagaaaaca caaagacatt
1501 agaataccta ggttcactgg gggtgtatgg ggtagatggg tggagaggga ggggataaga
1561 gaggtgcatg ttggtattta aagtagtgga ttcaaagaac ttagattata aataagagtt
1621 ccattaggtg atacatagat aagggctttt tctccccgca aacaccccta agaatggttc
1681 tgtgtatgtg aatgagcggg tggtaattgt ggctaaatat ttttgtttta ccaagaaact
1741 gaaataattc tggccaggaa taaatacttc ctgaacatct taggtctttt caacaagaaa
1801 aagacagagg attgtcctta agtccctgct aaaacattcc attgttaaaa tttgcacttt
1861 gaaggtaagc tttctaggcc tgaccctcca ggtgtcaatg gacttgtgct actatatttt
1921 tttattcttg gtatcagttt aaaattcaga caaggcccac agaataagat tttccatgca
1981 tttgcaaata cgtatattct ttttccatcc acttgcacaa tatcattacc atcacttttt
2041 catcattcct cagctactac tcacattcat ttaatggttt ctgtaaacat ttttaagaca
2101 gttgggatgt cacttaacat tttttttttt tgagctaaag tcagggaatc aagccatgct
2161 taatatttaa caatcactta tatgtgtgtc gaagagtttg ttttgtttgt catgtattgg
2221 tacaagcaga tacagtataa actcacaaac acagatttga aaataatgca catatggtgt
2281 tcaaatttga acctttctca tggatttttg tggtgtgggc caatatggtg tttacattat
2341 ataattcctg ctgtggcaag taaagcacac tttttttttc tcctaaaatg tttttccctg
2401 tgtatcctat tatggatact ggttttgtta attatgattc tttattttct ctcctttttt
2461 taggatatag cagtaatgct attactgaaa tgaatttcct ttttctgaaa tgtaatcatt
2521 gatgcttgaa tgatagaatt ttagtactgt aaacaggctt tagtcattaa tgtgagagac
2581 ttagaaaaaa tgcttagagt ggactattaa atgtgcctaa atgaattttg cagtaactgg
2641 tattcttggg ttttcctact taatacacag taattcagaa cttgtattct attatgagtt
2701 tagcagtctt ttggagtgac cagcaacttt gatgtttgca ctaagatttt atttggaatg
2761 caagagaggt tgaaagagga ttcagtagta cacatacaac taatttattt gaactatatg
2821 ttgaagacat ctaccagttt ctccaaatgc cttttttaaa actcatcaca gaagattggt
2881 gaaaatgctg agtatgacac ttttcttctt gcatgcatgt cagctacata aacagttttg
2941 tacaatgaaa attactaatt tgtttgacat tccatgttaa actacggtca tgttcagctt
3001 cattgcatgt aatgtagacc tagtccatca gatcatgtgt tctggagagt gttctttatt
3061 taaagtt ttaatttagt ataaacat
```

Modified oligonucleotides may comprise, for example, one or more of the following selected components: a modified internucleoside linkage, for example, a phosphorothioate linkage, and/or a modified sugar moiety, for example, a conformationally-strained sugar, for example, a Linked Nucleic Acid (LNA) or Bridged Nucleic Acid (BNA).

The chemical modification of the antisense polynucleotides disclosed or referenced herein may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (MBOs) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

In some embodiments, an antisense polynucleotide disclosed or referenced herein may include an oligonucleotide sugar moiety that is a modified sugar moiety. In some embodiments, the modified sugar moiety can be a sugar moiety which is a conformationally-strained sugar. In some embodiments, the conformationally-strained sugar can be a locked nucleotide (locked nucleic acid, or LNA). In some embodiments, the locked nucleotide can be selected from one of the following types: 2′-OCH₂-4′ (oxy-LNA), 2′-CH₂—CH₂-4′ (methylene-LNA), 2′-NH—CH₂-4′ (amino-LNA), 2′-N(CH₃) CH₂-4′ (methylamino-LNA), 2′-S—CH₂-4′ (thio-LNA), and 2′-Se—CH₂-4′ (seleno-LNA). In some embodiments, the conformationally-strained sugar can be a bridged nucleic acid (BNA). Some conformationally-strained sugar can be a locked nucleic acid are shown in Formula III and Formula IV in U.S. Pat. No. 10,465,188.

In some embodiments, antisense compounds comprise antisense polynucleotides comprising from 8 to about 80 nucleotides of an connexin extracellular loop, intracellular region, C-terminus or other region for modulating expression of one or more connexins selected from the group consisting of connexin 26, connexin 30, connexin 30.3, connexin 31, connexin 31.1, connexin 32, connexin 43, connexin 45, connexin 50 and connexin 58. The polynucleotides include synthesized polynucleotides having a length of less than 80 nucleotides, e.g., from 12-18 to about 50-80 nucleotides, preferably about 30 nucleotides or less, e.g., from 12 to about 30 nucleotides, and more preferably from about 15 to about 30 nucleotides. In one example, the polynucleotide has 30 nucleotides. In some embodiments, antisense compounds comprise modified or unmodified epithelial, endothelial, and/or vascular endothelial connexin antisense polynucleotides.

In some embodiments of this invention, the connexin 43 or other antisense oligonucleotide or polynucleotide has at least about 80%, 85%, 90%, 95%, 97%, 98% or 99% homology to a polynucleotide having a sequence selected from SEQ ID NOs: 1 to 17. Connexin modulators that are oligonucleotides or polynucleotides may have at least about 80%, 85%, 90%, 95%, 97%, 98%, or 99% homology to an 8 to 80 nucleotide portion of their respective sequences.

TABLE I
List of some connexin 43 antisense oligonucleotide embodiments

| | SEQ ID | | |
|---|---|---|---|
| ASN code | NO.for ASN | | ASN target site |
| name | sequence | ASN sequence | from 5′ end |
| | SEQ ID NO: 1 | GTAATTGCGGCAAGAAGAA | |
| | | TTGTTTCTGTC | |
| | SEQ ID NO: 2 | GTAATTGCGGCAGGAGGAA | |
| | | TTGTTTCTGTC | |
| | SEQ ID NO: 3 | GGCAAGAGACACCAAAGAC | |
| | | ACTACCAGCAT | |
| 24501 | SEQ ID NO: 5 | ACCCATGTTGCCTGGGCACC | 237-256 |
| 30004 | SEQ ID NO: 6 | GTAGGCTTGAACCTTGTCAA | 281-300 |
| 37503 | SEQ ID NO: 7 | TCTCCCCAGGCTGACTCAAC | 372-389 |
| LP2/37501 | SEQ ID NO: 4 | CCAGGCTGACTCAACCGCT | 368-385 |
| | | G | |
| 47001 | SEQ ID NO: 8 | CAGAAGCGCACATGAGAGA | 464-483 |
| | | T | |
| 47002 | SEQ ID NO: 9 | GAAGCGCACATGAGAGATT | 462-481 |
| | | G | |
| 50501 | SEQ ID NO: 10 | AGTGTGGGTACAGACACAA | 500-519 |
| | | A | |
| LP3 | SEQ ID NO: 11 | CAGACACAAATATGATCTG | 490-509 |
| | | C | |
| 50506 | SEQ ID NO: 12 | ATATGATCTGCAGGACCCA | 481-500 |
| | | G | |
| 112304 | SEQ ID NO: 13 | GTAATTGCGGCAAGAAGAA | 1134-1153 |
| | | T | |
| 1233 | SEQ ID NO: 14 | AGGCTGTGCATGGGAGTTA | 1233-1252 |
| | | G | |
| 133704 | SEQ ID NO: 15 | CGCTGGTCCACAATGGCTA | 1316-1335 |
| | | G | |
| 133705 | SEQ ID NO: 16 | GCTGGCTCTGCTTGAAGGTC | 1335-1354 |

Table I lists the polynucleotide sequences of several embodiments of connexin 43 polynucleotide modulators useful in the methods of the invention. When sequences such as SEQ ID NO:1-16 are noted, they and other Cx43 and other connexin antisense compounds represent both modified and unmodified oligonucleotides or polynucleotides. In some embodiments, the linkages between the nucleotides, and the structure of the sugar moiety of the nucleotides may be modified. In some embodiments, the internucleoside linkage between any two nucleotides can be a standard phosphodiester linkage. In some embodiments, the internucleoside linkage between any two nucleotides can be a phosphorothioate linkage. For example, SEQ ID NO:1 can be one of the following selected structures: GₛTₛAₛAₛTTGCGGCAAGAAGAATTGTTTCₛTSGₛTₛC, wherein “ₛ” denotes a phosphorothioate linkage between the two nucleotides. As another non-limiting example, SEQ ID NO:1 can be (G)(T)(A)(A)TTGCGGCAAGAAGAATTGTTTC(T)(G)(T)(C), wherein the parenthetical nucleotides have modified sugar moieties, as described below. In some embodiments the Cx43 antisense compounds may be modified by substituting one or more uridine nucleotides residues for one or more thymine nucleotides in SEQ ID NOs:1-17 or in the sequence of another connexin (e.g., Cx26, C32, Cx45, etc.).

Certain connexin modulators, including for example connexin 43 modulators, provide downregulation of connexin expression (for example, by downregulation of mRNA transcription or translation) or otherwise decrease or inhibit the activity of the connexin protein, connexin hemichannels or gap junctions. In the case of downregulation, this will have the effect of reducing direct cell-cell communication by gap junctions, or exposure of cell cytoplasm to the extracellular space by hemichannels, at the site at which connexin expression is downregulated.

In some embodiments, an anti-connexin antisense compound prevents, decreases or alters the activity or function of a hemichannel or a gap junction. As described herein, modulation of the gap junction activity or function by anti-connexin antisense compounds can lead to the closing of gap junctions, closing of hemichannels, and/or passage of molecules or ions through gap junctions and/or hemichannels.

Connexin modulators may also comprise one or polynucleotides selected, for example, from the group consisting of morpholino oligonucleotides, RNAi molecules, siRNA molecules, PNA molecules, DNAzymes, and 5′-end-mutated U1 small nuclear RNAs, and analogs of the preceding. These and other compounds may be used alone or in combination with one more connexin modulators.

Synthesis of antisense polynucleotides and other anti-connexin polynucleotides such as RNAi, siRNA, and ribozyme polynucleotides as well as polynucleotides having modified and mixed backbones can be performed. See e.g. Stein C. A. and Krieg A. M. (eds), Applied Antisense Oligonucleotide Technology, 1998 (Wiley-Liss).

The antisense polynucleotide may inhibit transcription and/or translation of a connexin protein (e.g. connexin 43). The antisense polynucleotide is generally antisense to connexin protein mRNA, for example, connexin 43. Such a polynucleotide may be capable of hybridizing to connexin protein mRNA and may thus inhibit the expression of connexin by interfering with one or more embodiments of connexin protein mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases. Preferably the polynucleotide is a specific inhibitor of transcription and/or translation from the connexin 43 gene or mRNA, and does not inhibit transcription and/or translation from other genes or mRNAs.

The connexin modulator product may bind to the connexin 43 gene or mRNA either (i) 5′ to the coding sequence, and/or (ii) to the coding sequence, and/or (iii) 3′ to the coding sequence. The antisense polynucleotide may hybridize to part of the connexin protein mRNA, such as connexin 43 mRNA. Typically, the antisense polynucleotide hybridizes to the ribosome binding region or the coding region of the connexin protein mRNA. The polynucleotide may be complementary to a region of the connexin mRNA. For example, the polynucleotide may be the exact complement of a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention. Thus, the polynucleotide is typically a homologue of a sequence complementary to the mRNA. The polynucleotide may be a polynucleotide which hybridizes to the connexin protein mRNA under conditions of medium to high stringency such as 0.03M sodium chloride and 0.03M sodium citrate at from about 50° C. to about 60° C.

Antisense polynucleotides may be part of compositions which may comprise polynucleotides to more than one connexin protein. Preferably, one connexin protein to which polynucleotides are directed is connexin 43. Others include connexins found in the ocular and/or corneal epithelium. Some embodiments of the invention are described with reference to oligodeoxynucleotides. However, other suitable polynucleotides (such as RNA polynucleotides) may be used.

### Other Connexin Modulators

In addition to connexin antisense (e.g. SEQ ID NO:1), connexin peptidomimetics (e.g., Peptide5, XG19, etc.) and connexin hemichannel antagonists (e.g. tonabersat), connexin binding proteins, including antibodies, antigen-binding antibody fragments, and the like, are also suitable connexin modulators for use in methods of the invention and dosed with a therapeutically effective amount according to the one or more of the dosing schedules described herein. Binding proteins include, for example, monoclonal antibodies, polyclonal antibodies, antibody fragments (including, for example, Fab, F(ab′)2 and Fv fragments; single chain antibodies; single chain Fvs; and single chain binding molecules such as those comprising, for example, a binding domain, hinge, CH2 and CH3 domains, recombinant antibodies, and antibody fragments which are capable of binding an antigenic determinant (i.e., that portion of a molecule, generally referred to as an epitope) that makes contact with a particular antibody or other binding molecule. These binding proteins, including antibodies, anti-binding antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g., a connexin protein or connexin hemichannel epitope). Methods of synthesizing antibodies and binding fragments as well as peptides and polypeptides, including peptidomimetics and peptide analogs can also be performed using suitable methods. See e.g. Lihu Yang et al., Proc. Natl. Acad. Sci. U.S.A., 1; 95(18): 10836-10841 (Sep. 1 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

Connexin modulators also include antibodies and binding fragments (e.g. scFvs, human V_{H} or V_{L} domains, humanized camelid VHH domains, Ig_{NAR} single domains, etc.) thereof that bind connexin protein, and connexin peptides and polypeptides, including peptidomimetics and peptide analogs of connexin that modulate hemichannel or gap junction activity or function, and other gap junction blocking agents and gap junction protein phosphorylating agents. Connexin protein peptides and polypeptides may, for example, bind to connexin protein to inhibit its function, or may inhibit connexin function by mimicking regions of connexin protein to inhibit or disrupt its binding to other gap junction proteins. Strategies known in the art may be used to improve the naturally short half-life of antibody fragments, including PEGylation, the use of repeating peptide sequences, polysialylation, albumin or IgG binding or fusions, and other approaches.

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹¹ M⁻¹, about 10¹¹ M⁻¹, and about 10¹² M⁻¹. Affinities of binding proteins useful to treat subjects according to the present invention can be readily determined using conventional techniques, for example those described by Scatchard, et al., 1949 Ann. N.Y. Acad. Sci. 51: 660.

Other compounds that may be used for modulating, blocking or closing gap junctions (e.g. phosphorylating connexin 43 tyrosine and/or serine residue) are known and have been reported in U.S. Pat. Nos. 7,153,822 and 7,250,397, for example.

### Pannexin Modulators

Any modulator that is capable of eliciting a desired inhibition of the passage (e.g., transport) of molecules through a pannexin channel (e.g., ATP), including a pannexin 1 channel, for example, may be used in embodiments of the invention and dosed according to the one or more of the schedules described herein. Such compounds include, for example, binding proteins (e.g. scFvs, antibodies, etc.), polypeptides (e.g. peptidomimetics), polynucleotides (e.g., antisense compounds) and organic compounds (e.g. probenecid and compounds of Formula III) and/or prodrugs thereof that can, for example, block the function or activity of a pannexin channel in whole or in part (e.g. by modulating release of ATP from a pannexin channel). See, e.g., Caufriez, A, et al. Determination of Structural Features That Underpin the Pannexin1 Channel Inhibitory Activity of the Peptide (10)Panx1. Bioorg. Chem. 2023, 138:106612. See also Van Campenhout, R, et al. Nanobody-based pannexin1 channel inhibitors reduce inflammation in acute liver injury. J Nanobiotechnol 21, 371 (2023); Lissoni, A, et al. Cx43 Hemichannel and Panx1 Channel Modulation by Gap19 and ¹⁰Panx1 Peptides. Int. J. Mol. Sci. 2023, 24, 11612; Lamouroux, A, et al. Structure-Based Design and Synthesis of Stapled ¹⁰Panx1 Analogues for Use in Cardiovascular Inflammatory Diseases J. Med. Chem. 2023 66:13086-13102.

Compounds for pannexin modulation can be any compound of Formula III:

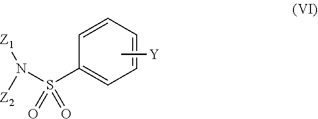

wherein
Z₁ and Z₂ are independently selected from: (C₁-C₄)alkyl, halo(C₁-C₄)alkyl, (C₁-C₄)alkoxy, halo(C₁-C₄), (C₁-C₄)cycloalkyl, (C₁-C₄)alkyl-S—, (C₁-C₄)alkylamino, (C₁-C₄)cycloalkylamino, di(C₁-C₄)alkylamino, and amino(C₁-C₄)alkyl; Y is selected from: hydrogen, halo, cyano, hydroxy, (C₁-C₄)alkyl, halo(C₁-C₄)alkyl, (C₁-C₄)alkoxy, halo(C₁-C₄)alkoxy, cyano(C₁-C₄)alkyl, amino, (C₁-C₄)alkylamino, di(C₁-C₄)alkylamino, amino(C₁-C₄)alkyl, (C₁-C₄)alkylamino(C₁-C₄)alkyl, di[(C₁-C₄)alkyl]amino(C1-C4)alkyl, trifluoromethylthio, hydroxy(C₁-C₄)alkyl, (C₁-C₄)alkoxy(C₁-C₄)alkyl, —C(O)R₁, C(O)OR₁, —OC(O)R₁, —C(O)—N(R₁)₂, —CH₂—C(O)R₁, —CH₂—C(O)OR₁, —CH₂—OC(O)R₁, —CH₂—C(O)—N(R₁)₂, S(O)₂R₁, S(O)₂N(R₁)₂, (C₃-C₅)cycloalkyl, (C₃-C₈)cycloalkyl(C₁-C₄)alkyl, SO₃H, and SO₄H;
R₁ is selected from: hydrogen, NH₂, NR₂R₃, OH, CH₂OH, and CH₂CH₂OH;
R2 and R3 are independently selected from: hydrogen, (C₁-C₄)alkyl, (C₁-C₄)alkoxy, and (C₁-C₄)cycloalkyl; and,
halo is chlorine, bromine, iodine, or fluorine.

One Formula III compound for pannexin modulation is probenecid, shown below:

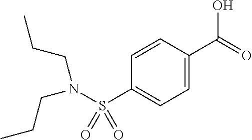 [SMILES: CCCN(CCC)S(=O)(=O)c1ccc(C(=O)O)cc1]

Probenecid may be known by the IUPAC name p-[Dipropylsulfamoyl]benzoic acid and has the structure shown above.

In certain embodiments, probenecid and/or an analogue thereof are nonionic, are in the form of a free base, a free acid, or a pharmaceutically acceptable salt. By way of example, a pharmaceutically acceptable salt includes a hydrochloride salt and salts derived from acid including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid, a salt of itself. In one embodiment, the salt is a hydrochloride salt. In other embodiments, one or more polymorph, one or more isomer, and/or one or more solvate of probenecid and/or an analogue thereof may be used.

In some embodiments, the pannexin modulators can include or exclude pannexin peptidomimetics. Pannexin peptide sequences can comprise about 8 to about 40 (e.g., 40-80) consecutive amino acids of an extracellular domain, an intracellular domain, a carboxy terminus part, or an amino terminus part, of the polypeptides Panx1, Panx2 or Panx3. Pannexin modulators may comprise, in some embodiments, a portion of an extracellular loop of Panx 1, 2 or 3. In some embodiments, a pannexin peptidomimetic can comprise about 8 to about 40 consecutive amino acids of an extracellular domain, an intracellular domain, a carboxy terminus part, or an amino terminus part, of pannexin 1. In some embodiments, for example, the pannexin modulator may comprise, for example, the Panx1 mimetic blocking peptide is ¹⁰Panx1 (WRQAAFVDSY (SEQ ID NO: 21)). In some embodiments, the Panx1 mimetic peptide is a stapled ¹⁰Panx1 analogue (see, e.g., Lamouroux, A, et al. (2023), supra.

In some embodiments, the mimetic peptide Gap19 may be used to modulate pannexin 1 channels. In some embodiments, ¹⁰Panx1 may be used to modulate connexin 43 hemichannels. See Lissoni, A, et al. (2023) supra.

In some embodiments, pannexin mimetic modulators can comprise, for example, about 40 to about 80 (including from 8-40) consecutive amino acids. They may comprise part of an extracellular domain, an intracellular domain, a carboxy terminus, or an amino terminus of the Panx1 peptide, the Panx2 peptide and/or the Panx3 peptide, or variants thereof. The sequence of the Panx1 polypeptide is known and can be found, for example, at U.S. Pat. No. 10,465,188 (also published as WO2016029191) as SEQ ID NO: 122 therein (also identified as NCBI RefSeq: NP_056183.2) at Col 42, lines 35-50 therein, The sequence of the Panx2 polypeptide is known and can be found, for example, at U.S. Pat. No. 10,465,188 as SEQ ID NO: 123 therein (Panx2 peptide, also identified as RefSeq NP_443071.2) at Col 42 line 51 to Col. 43, line 9 therein. The sequence of the Panx3 polypeptide is known and can be found, for example, at U.S. Pat. No. 10,465,188 as SEQ ID NO: 124 therein Panx3 peptide, also identified as RefSeq NP_443191.1) at Col. 43, line 10 to Col. 44, line 12 therein. IN some embodiments, the pannexin peptidomimetic is a pannexin 1 peptidomimetic.

In some embodiments the pannexin modulators that are oligonucleotides or polynucleotides may have at least about 80%, 85%, 90%, 95%, 97%, 98%, or 99% homology to an 8 to 80 nucleotide portion of Panx1 nRNA (Panx1 polynucleotide RefSeq ID NM_015368.3), Panx2 mRNA (Panx2 polynucleotide RefSeq ID NM_052839.3 for variant 1; RefSeq ID NM_001160300.1 for Panx2 polynucleotide variant 2; RefSeq ID NR_027691.1 for Panx2 polynucleotide variant 3), or Panx3 mRNA (Panx3 polynucleotide RefSeq ID NM_052959.2). The sequence of the Panx1 mRNA is known and can be found, for example, at U.S. Pat. No. 10,465,188 (also published as WO2016029191) as SEQ ID NO: 117 therein at Col. 44, line 15 to Col. 45, line 34. The sequence of the Panx2, variant 1 mRNA is known and can be found, for example, at U.S. Pat. No. 10,465,188 as SEQ ID NO: 118 therein at Col. 45, line 36 to Col. 47, line 58. The sequence of the Panx2, variant 2 mRNA is known and can be found, for example, at U.S. Pat. No. 10,465,188 as SEQ ID NO: 119 therein at Col. 47, line 60 to Col. 49. The sequence of the Panx2, variant 3 mRNA is known and can be found, for example, at U.S. Pat. No. 10,465,188 as SEQ ID NO: 120 therein at Col. 51, line 1 to Col. 53, line 22. The sequence of the Panx3 mRNA is known and can be found, for example, at U.S. Pat. No. 10,465,188 as SEQ ID NO: 121 therein at Col. 53, line 24 to Col. 53, end of page. Some useful pannexin 1 antisense sequences include 5′-TATGCAGCCACAGTGGGAGG-3′ (685-704) (SEQ ID NO: 247) and 5′-TCAGATACCTCCCACAAACT-3′ (929-948) (SEQ ID NO: 248), 5′-TAGCACCTGCCAGTCCAGAAT-3′ (SEQ ID NO: 249), and 5′-GGTCCAGGTCCATCTCTCAGG-3′ (SEQ ID NO: 250), and may be made, for example, using unmodified nucleotides or produced as phosphorothioate ODNs.

### Inflammasome Modulators

The NLRP3 inflammasome is currently the best characterized. It comprises NLRP3, ASC (PYCARD) and procaspase-1 (and CARD8 (Cardinal) may also be a component). It is activated by a number of pathogens and bacterial toxins as well as diverse PAMPs, danger-associated molecular patterns (DAMPS). Multiple studies have shown that activation of the NLRP3 inflammasome by particulate activators (e.g. Hornung V, et al. Silica crystals and aluminum salts activate the NALP3 inflammasome through phagosomal destabilization. Nat Immunol. 2008 9(8):847-56) requires phagocytosis, but this is not required for the response to ATP, which is mediated by the P2X7 receptor (Kahlenberg J M, Dubyak G R. Mechanisms of caspase-1 activation by P2X7 receptor-mediated K+ release. Am J Physiol Cell Physiol. 2004 286(5):C1100-8) and appears to involve the pannexin membrane channel (Pelegrin P, Surprenant A. Pannexin-1 mediates large pore formation and interleukin-Ibeta release by the ATP-gated P2X7 receptor. EMBO J. 2006 25(21):5071-82).

Any modulator that is capable of eliciting a desired inhibition of an inflammasome (e.g., the NLRP3 inflammasome), either directly or indirectly, may be used in compositions and methods of the invention. Such compounds include, for example, binding proteins (e.g. scFvs, antibodies, etc.), polypeptide modulators (e.g. peptidomimetics), polynucleotide modulators (e.g., antisense compounds) and organic compounds useful to modulate an inflammasome, whether now known or later developed, including those described herein. In some embodiments, inflammasome modulators (e.g., NLRP3 inflammasome modulators) useful in methods, compositions and kits of the invention, including to reverse or alleviate one or more signs and/or symptoms of a post-acute infection syndrome (e.g., long COVID, long flu, post-treatment Lyme disease syndrome, etc.) may be referred to as “direct” or “indirect” inflammasome modulators. Useful inflammasome modulators that may be referred to as direct inflammasome modulators include, for example, oridonin, MCC950, tranilast and analogues thereof which directly binds to the NACHT domain of NLRP3 and change its conformation; OLT1177 and parthenolide which suppress the ATPase activity of NLRP3; CY-09 which binds to the NACHT domain and inhibits ATPase of NLRP3; BAY11-7082 and VI-16 which block the binding between TXNIP and NLRP3; NIC7 (NLRP3-inhibitory compound), and its derivatives which inhibit NLRP3-mediated activation of caspase 1 along with the secretion of interleukin (IL)-1β, IL-18 and lactate dehydrogenase. Useful inflammasome modulators that may be referred to as indirect inflammasome modulators include, for example, glyburide, 1673-34-0, FC11A-2, β-hydroxybutyrate, etc., and modulators of ATP-induced inflammasome activation such as tonabersat and other small molecule connexin hemichannel modulators, connexin peptidomimetics including for example, Peptide5, XG19, etc. In some embodiments, the inflammasome modulators are NLRP3 inflammasome modulators. In some embodiments, the inflammasome modulators are direct NLRP3 inflammasome modulators. In some embodiments, the inflammasome modulators are indirect NLRP3 inflammasome modulators. In some embodiments, the inflammasome modulators (e.g., NLRP3 inflammasome modulators) are used in and useful for methods, compositions and kits of the invention relating to reversing or alleviating one or more signs and/or symptoms of a post-acute infection syndrome (e.g., long COVID, long flu, post-treatment Lyme disease syndrome, etc.).

### Connexin, Pannexin and Inflammasome Modulator Combinations and Combined Administrations

In some embodiments, methods, uses and compositions of the invention may comprise the use of a combination of one or more connexin hemichannel (e.g., connexin 32 hemichannel) modulators (e.g., tonabersat), for example, one or more connexin antisense modulators (e.g. SEQ ID NO:1), and/or one or more connexin peptidomimetics (e.g. Peptide5, Gap19, XG19), and/or small molecule connexin hemichannel modulators (e.g. compounds of Formula I, such as tonabersat and/or prodrugs thereof, including compounds of Formula II). Two or more connexin hemichannel modulators may be administered alone or together. In some embodiments, two or more separate pharmaceutical compositions that each contain one or more connexin hemichannel modulators are provided for administration. Pharmaceutical compositions are also provided for co-administration in the form of a combined preparation, for example, as an admixture of two or more connexin hemichannel modulators, which may be modified or unmodified, or in the form of prodrugs, as the case may be, for example one or more small molecule hemichannel blockers or inhibitors, one or more connexin hemichannel modulator polynucleotides, and optionally one or more connexin hemichannel modulator peptidomimetics, that can reduce hemichannel opening, activity, function, etc. In some embodiments, the two or more connexin hemichannel modulators are directed to different connexins (e.g., connexin 26 and connexin 43, connexin 43 and connexin 45, and so on). In some embodiments, the two or more connexin hemichannel modulators are directed to the same connexin (e.g., connexin 26 or connexin 37 or connexin 43 or connexin 45, etc.).

In some embodiments, methods, uses and compositions of the invention may comprise the use of a combination of two or more inflammasome modulators, for example, a direct inflammasome modulator (e.g., an organic compound, antisense and/or peptidomimetic inflammasome modulator), an indirect inflammasome connexin modulators (e.g. a connexin hemichannel modulator, for example, compounds of Formula I, such as tonabersat and/or prodrugs thereof, including compounds of Formula II), or a pannexin channel modulator (e.g., an organic compound, antisense and/or peptidomimetic pannexin modulator). Two or more inflammasome modulators may be administered alone or together. In some embodiments, two or more separate pharmaceutical compositions that each contain one or more inflammasome modulators are provided for administration. Pharmaceutical compositions are also provided for co-administration in the form of a combined preparation, for example, as an admixture of two or more different inflammasome modulators, which may be modified or unmodified, or in the form of prodrugs, as the case may be, for example one or more small molecule inflammasome modulators or inhibitors, one or more inflammasome modulator polynucleotides, and optionally one or more inflammasome modulator peptidomimetics. In some embodiments, the two or more inflammasome modulators are directed to the same inflammasome (e.g., NLRP3, etc.). In some embodiments, the two or more inflammasome modulators are directed to the different parts of the same inflammasome (e.g., different parts of the NLRP3 inflammasome).

In some embodiments, methods, uses and compositions of the invention may comprise the use of a combination of two or more pannexin modulators, for example, an organic compound, peptidomimetic and/or antisense pannexin modulator (e.g., pannexin 1 modulators, or any combination of pannexin 1, 2 and/or 3 modulators). Two or more inflammasome modulators may be administered alone or together. In some embodiments, two or more separate pharmaceutical compositions that each contain one or more pannexin (e.g., pannexin 1) modulators are provided for administration. Pharmaceutical compositions are also provided for co-administration in the form of a combined preparation, for example, as an admixture of two or more different pannexin modulators, which may be modified or unmodified, or in the form of prodrugs, as the case may be, for example one or more small molecule pannexin modulators or inhibitors, one or more pannexin modulator polynucleotides, and optionally one or more pannexin modulator peptidomimetics.

In some embodiments, one or more connexin hemichannel, pannexin and/or inflammasome modulators may be administered alone or together. In some embodiments, two or more separate pharmaceutical compositions that each contain one or more connexin hemichannel, pannexin and/or inflammasome modulators are provided for administration. Pharmaceutical compositions are also provided for co-administration in the form of a combined preparation, for example, as an admixture of two or more connexin hemichannel, pannexin and/or inflammasome modulators.

Treatment of a subject or a patient, including e.g., for one or more of the diseases, disorders, defects or conditions described or referenced herein, with one or more pharmaceutical compositions of the invention, e.g., a connexin hemichannel modulator, or a first connexin hemichannel modulator agent and a second, different connexin hemichannel modulator agent (which may be directed to the same or different connexins), may comprise their simultaneous, separate, sequential or sustained administration. Likewise, treatment of a subject or a patient, including e.g., for one or more of the diseases, disorders, defects or conditions described or referenced herein, with one or more pharmaceutical compositions of the invention, e.g., an inflammasome modulator, or a first inflammasome modulator agent and a second, different inflammasome modulator agent (which may be directed to the same or different inflammasomes), may comprise their simultaneous, separate, sequential or sustained administration. Similarly, treatment of a subject or a patient, including e.g., for one or more of the diseases, disorders, defects or conditions described or referenced herein, with one or more pharmaceutical compositions of the invention, e.g., a pannexin channel modulator, or a first pannexin channel modulator agent and a second, different pannexin channel modulator agent (which may be directed to the same or different pannexin channels), may comprise their simultaneous, separate, sequential or sustained administration.

The term “a combined preparation” includes a “kit of parts” or “article of manufacture” in the sense that the combination partners as defined above can be dosed independently or by use of different fixed combinations with distinguished amounts of the combination partners (a) and (b), i.e. simultaneously, separately or sequentially, whether in pharmaceutical form (e.g. topical or oral) or dressing/matrix form (e.g. modulator-impregnated) or both in accordance with the methods of the invention. The parts of the kit can then, for example, be administered simultaneously or chronologically staggered, that is at different time points and with equal or different time intervals for any part of the kit of parts.

In some embodiments, a combined preparation is administered, wherein two or more separate modulator compositions are administered to a subject in accordance with the methods invention. In some embodiments, the first composition comprises a therapeutically effective amount of modulator, such as a connexin hemichannel modulator, e.g., an anti-connexin 43 polynucleotide, peptide, or peptidomimetic, or a hemichannel modulating compound, and the second composition comprises a therapeutically effective amount of a second modulator, such as a connexin hemichannel modulator, e.g., an anti-connexin 26, 37, 43 or 45 polynucleotide, peptide, or peptidomimetic, a hemichannel closing compound and/or an ocular treatment agent. In another embodiment a third composition is administered comprising one or more connexin hemichannel modulators or another therapeutic agent (e.g., for treating one or more signs and/or symptoms of a post-acute infection syndrome (e.g., long COVID, long flu, post-treatment Lyme disease syndrome, etc.). In some embodiments, the first composition comprises a therapeutically effective amount of modulator, such as an inflammasome modulator, e.g., an NLRP3 modulator, and the second composition comprises a therapeutically effective amount of a second inflammasome modulator. In another embodiment, a third composition is administered comprising one or more modulators and/or another treatment agent. In some embodiments, the first composition comprises a therapeutically effective amount of modulator, such as a pannexin channel modulator, e.g., a pannexin 1 channel modulator, and the second composition comprises a therapeutically effective amount of a second pannexin channel modulator. In another embodiment, a third composition is administered comprising one or more pannexin channel modulators and/or another treatment agent. In some embodiments, the compositions comprise one or more connexin hemichannel modulators, one or more inflammasome modulators, one or more pannexin channel modulators, or combinations of one or more modulators of one or more connexin hemichannels (including, e.g., connexin 43 hemichannels), inflammasomes (including, e.g., NLRP3 inflammasomes) and/or pannexin channels (including, e.g., pannexin 1 channels). The compositions may also contain another agent for treatment or prophylaxis.

The modulators and modulators compositions can be dosed, administered, or formulated in methods of the invention as described herein.

### Doses and Dose Regimens

Examples of effective doses that may be used for the treatment of a post-acute infection syndrome and/or one or more signs and/or symptoms thereof, including those described or referenced herein, are described. They include, for example, doses and dose regimens that are useful for the application or administration of connexin hemichannel modulators (e.g., connexin 43 hemichannel modulators), pannexin channel modulators (e.g. pannexin 1 channel modulators), direct inflammasome modulators and indirect inflammasome modulators (e.g., direct or indirect NLRP3 inflammasome modulators).

In some embodiments, a therapeutically or prophylactically effective amount of a modulator effective in methods of the invention comprises a composition that includes certain doses of connexin hemichannel modulator (for example a connexin 43 hemichannel modulator), an inflammasome modulator (for example an NLRP3 inflammasome modulator) and/or a pannexin channel modulator (for example a pannexin 1 channel modulator).

In some embodiments, the connexin hemichannel modulator dose or dose in a composition comprises a small molecule, organic or chemical connexin hemichannel modulator (e.g., tonabersat, carabersat, or another compound according to Formula I or a compound according to Formula II) dose. In some embodiments, the connexin hemichannel modulator dose or dose included in a composition comprises a connexin peptidomimetic (e.g., Peptide5, Gap19, XG19, Gap26, Gap27, aCT1, etc.) dose. In some embodiments, the connexin hemichannel modulator dose or dose comprising a composition comprises a connexin antisense molecule (e.g. SEQ ID NO:1) dose. In some embodiments, the connexin modulator is a connexin 43 hemichannel modulator.

In some embodiments, the inflammasome modulator dose or dose in a composition comprises a small molecule, organic or chemical pannexin channel modulator dose. In some embodiments, the inflammasome modulator dose or dose included in a composition comprises an inflammasome peptidomimetic dose. In some embodiments, the inflammasome modulator dose or dose comprising a composition comprises an inflammasome antisense molecule dose. In some embodiments, the inflammasome modulator is an NLPR3 inflammasome modulator.

In some embodiments, the pannexin channel modulator dose or dose in a composition comprises a small molecule, organic or chemical pannexin channel modulator (e.g., a compound according to Formula III) dose. In some embodiments, the pannexin channel modulator dose or dose included in a composition comprises a pannexin channel peptidomimetic (e.g., 10Panx1) dose. In some embodiments, the pannexin channel modulator dose or dose comprising a composition comprises a pannexin channel antisense molecule dose. In some embodiments, the pannexin channel modulator is a pannexin 1 channel modulator.

In some embodiments, the dose of a connexin hemichannel modulator, inflammasome modulator and/or a pannexin channel modulator is measured in milligrams (mg). In some embodiments, the dose of a connexin hemichannel modulator, inflammasome modulator and/or a pannexin channel modulator is measured in milligrams per kilogram (mg/kg) of a subject or patient. In some embodiments, the dose is measured in milligrams per kilogram per day (mg/kg/day). In some embodiments, the dose of a connexin hemichannel modulator, inflammasome modulator and/or a pannexin channel modulator is measured in milligrams per milliliter (mg/mL) or micrograms per milliliter (μg/mL). In some embodiments, the dose of a connexin hemichannel modulator, inflammasome modulator and/or a pannexin channel modulator is measured in micromolar (μM) or millimolar (mM) amounts of the modulator in in a composition to be administered, or as local, site-of-action or circulating concentration, including as described herein.

In some embodiments, the connexin hemichannel modulator being dosed comprises or consists essentially of a small molecule, organic or chemical connexin hemichannel modulator (e.g., tonabersat, carabersat, or another compound of Formula I or Formula II). In some embodiments, the connexin hemichannel modulator being dosed comprises or consists essentially of a peptidomimetic connexin hemichannel modulator (e.g., XG19). In some embodiments, the connexin hemichannel modulator being dosed comprises or consists essentially of an antisense connexin hemichannel modulator (e.g., SEQ ID NO:1). In some embodiments, the inflammasome modulator being dosed comprises or consists essentially of a small molecule, organic or chemical inflammasome modulator. In some embodiments, the inflammasome modulator being dosed comprises or consists essentially of a peptidomimetic inflammasome modulator. In some embodiments, the inflammasome modulator being dosed comprises or consists essentially of an antisense inflammasome modulator. In some embodiments, the pannexin channel modulator being dosed comprises or consists essentially of a small molecule, organic or chemical inflammasome modulator (e.g., probenecid or another compound according to Formula III). In some embodiments, the pannexin channel modulator being dosed comprises or consists essentially of a peptidomimetic pannexin channel modulator (e.g., ¹⁰Panx). In some embodiments, the pannexin channel modulator being dosed comprises or consists essentially of an antisense pannexin channel modulator.

In some embodiments, the doses may be administered to provide a therapeutically or prophylactically effective amount of a connexin hemichannel modulator, an inflammasome modulator and/or a pannexin channel modulator for treating or preventing one or more signs and/or symptoms of a post-acute infection syndrome, including those noted, described or referenced herein.

In some embodiments, the doses referenced herein are administered orally. In some embodiments, the doses referenced herein are administered by injection (e.g. intravenous (IV), subcutaneous (SC/SQ), intraperitoneal (IP), intradermal (ID), or intramuscular (IM)). In some embodiments, the doses are administered topically to a subject or a patient (e.g., to the skin or the eye). In some embodiments, the doses referenced herein are administered by other routes known in the art. Compounds disclosed herein or used as described herein may also be administered via implant, tissue or organ implant (e.g., hepatic, renal, cerebral, ocular, heart, CNS, etc.) or local administration including via a matrix, by inhalation or spray, sublingually, transdermally, via buccal administration, rectally, as an ophthalmic solution, ocular injection, intra-aortal, intracranial, subdermal, intraperitoneal, subcutaneous, transnasal, sublingual, intrathecal, or rectal or by other means, in dosage unit formulations containing conventional or other pharmaceutically acceptable carriers now known or later developed.

In some embodiments, a dose of a modulator or a composition or compositions containing one or more modulators is/are administered to a cell, a group of cells, a subject or a patient in a single dose, or in two or more divided doses, e.g., for one or more daily or weekly or other timed administrations. In some embodiments, modulator doses are administered at least once every day. In some embodiments, modulator doses are administered BID, TID or QID. In some embodiments, modulator doses are administered daily AC, every other day or every other evening. Reference to “administration” refers to administration of a modulator in a single dose or in divided doses. Wherever a dose of a particular modulator (e.g., a connexin hemichannel modulator or modulator composition, an inflammasome modulator or modulator composition and/or a pannexin channel modulator or composition) is not specified in a dose regimen or method of the invention for the treatment of a disease, disorder, defect or condition, dosing with a therapeutically effective amount of a modulator(s) or modulator composition(s) is intended.

The modulator dosages may be varied or may vary within one or more of the dose ranges or regimens provided depending upon the dosage form employed and the route of administration utilized.

For any modulators used in the method of the invention, the therapeutically effective dose can be evaluated, estimated and/or quantified using cell culture assays (e.g., ARPE-19 cells). Therapeutically effective doses of modulators can also be evaluated, estimated and/or quantified using tissue and/or assays with tissue (e.g., brain). Therapeutically effective doses of modulators can also be evaluated, estimated and/or quantified using animals (e.g., mice). Data obtained from cell culture assays, tissue assays, tissue experiments, and animal studies—as well as one or more of the methods for quantifying modulator activity can be used in identifying, evaluating or confirming a range of dosages for use in humans.

In some embodiments, a modulator dose may be formulated from cell cultures, tissue assays or experiments, and/or animal models to achieve a concentration or range of a modulator that includes the IC₅₀. IC₅₀ is a quantitative measure that indicates how much of a particular substance (e.g. a modulator drug, compound or composition) is needed to inhibit a given biological process or response or biological component by 50%. The biological component may be a protein, cell, cell receptor, etc. The biological process may be inflammasome activation, connexin hemichannel opening, or pannexin channel opening. In some embodiments, connexin hemichannel opening or pannexin channel opening may be measured by release of ATP. The IC₅₀ is the concentration of a substance where ATP release (e.g., from a connexin hemichannel or a pannexin channel) is reduced by half. In some embodiments, a modulator dose may be formulated from cell cultures, tissue assays or experiments, and/or animal models to achieve a concentration or range of a modulator that includes the EC₅₀. EC₅₀ is the concentration of a particular substance (e.g. a modulator drug, compound or composition) that gives half-maximal response. Half maximal effective concentration is a measure of the concentration of a modulator that induces a biological response halfway between the baseline and maximum after a specified exposure time. Such information can be used to accurately determine, confirm or refine doses useful in a subject or patient in a method or a use described or referenced herein. The dosage, IC₅₀ and EC₅₀ can be determined from the concentration of the amount administered, expected mass of the animal model tested (200-300 g per rat for adult Wistar rats), to determine the dose in units of mg/kg from concentration (micromolar) administered or amount (mg) administered. In humans and animals, the exact formulation, route of administration and dosage can be chosen by the individual physician in view of the patient's condition (see, e.g., Fingl et al., 1975, In: The Pharmacological Basis of Therapeutics, Ch. 1, p. 1.) but will be initially set by the FDA and/or other regulatory agencies following clinical trials.

It will be appreciated that the dose of modulator administered (e.g. a connexin hemichannel modulator, such as a compound of Formula I, for example tonabersat, or a compound according to Formula II, an inflammasome modulator, and a pannexin channel modulator, such as a compound of Formula III, e.g., probenecid, ¹⁰Panx1, etc.), the period of administration, and the general administration regime may differ between subjects depending on such variables as the target site to which it is to be delivered, the severity of any symptoms of a subject to be treated, the type of disorder to be treated, size of unit dosage, the mode of administration chosen, and the age, sex and/or general health of a subject and other factors known to those of ordinary skill in the art.

Modulator administration may include a single daily dose, administration of a number of discrete divided doses, or continuous administration, as may be appropriate. By way of example, unit doses may be administered once or more than once per day, for example 1, 2, 3, 4, 5 or 6 times a day to achieve a desired total daily dose. By way of example, a unit dose of a connexin hemichannel modulator, an inflammasome modulator, and/or a pannexin channel modulator may be administered in a single daily dose or a number of discrete doses, or continuously to achieve a daily dose of approximately about 10 to about 100 mg, about 100 to about 400 or 500 mg, about 40 to 80 or 100 mg, or any range between any two recited dosages or any dose between any two recited dosages.

In some embodiments, a connexin hemichannel modulator (e.g., a connexin 43 hemichannel modulator) is provided in an amount to achieve and/or to maintain a steady state amount over a desired period of time, including some or part of a dosing regimen for use in addressing one or more signs or symptom of a post-acute infection syndrome, disorder, disease or condition. In some embodiments, the connexin hemichannel modulator (e.g., a connexin 43 hemichannel modulator) is a compound according to Formula I. In some embodiments, the connexin hemichannel modulator (e.g., a connexin 43 hemichannel modulator) is a compound according to Formula II. In some embodiments, the connexin hemichannel modulator (e.g., a connexin 43 hemichannel modulator according to Formula I is tonabersat. Following single and repeat oral dosing, tonabersat was generally quantifiable in the plasma up to at least 96 hours post-dose. Maximal plasma concentrations generally occurred between 1 and 3 hours after dosing. Thereafter, plasma concentrations declined in an approximately bi-exponential manner. The terminal elimination phase T_{1/2} was approximately 24 to 33 hours. There was no evidence of time- or dose-related changes in systemic exposure of tonabersat. For a drug to show no change in its pharmacokinetic behavior on chronic dosing, the steady-state accumulation ratio (Rs) should equal unity. This was found to be the case for tonabersat, since the point estimates for Rs in each dose group (40 and 80 mg) were very close to unity, with the 95% CIs approximately (0.9, 1.1) for both dose groups. Although the terminal elimination half-life of tonabersat was slightly longer following repeated administration, the mean increase of approximately 3 hours is numerically small compared to the mean half-life of approximately 25 hours. The observed accumulation ratio (Ro) of tonabersat was also as predicted for a drug with an elimination half-life of about 1 to 1.5 days, dosed once daily for 7 days, with values indicative of average increases in systemic exposure of 2.24- and 2.71-fold for the 40 mg and 80 mg dose groups, respectively. A similar pattern was observed (in Cmax) with average increases of 1.71- and 2.43-:fold for the 40 mg and 80 mg dose groups, respectively. In accordance with its half-life of 1 to 1.5 days, steady state was achieved in most subjects, between 4 to 5 once daily doses of tonabersat as shown by the plateau in the plasma tonabersat concentrations taken immediately pre-dose (Cpre) on days 10 to 12. Systemic exposure of a single 40 mg of tonabersat, as judged by Cmax and AUC, were similar to results from a study where healthy subjects were administered a single oral dose of 40 mg tonabersat as a direct compression tablet. The pharmacokinetics of tonabersat were approximately dose proportional between 40 and 80 mg once daily, since there was no apparent trend in the dose-normalized (to 40 mg) Cmax and AUC(O-t) values for tonabersat following either single or multiple doses of tonabersat. This observation fully supported the linearity of the pharmacokinetics of tonabersat up to doses of 80 mg once daily, for example. Additionally, after single dosing with 240 mg and 320 mg tonabersat under fasted conditions, median tmax was 16 h and 10.5 h, respectively, whereas after single or multiple dosing with 240 mg to 400 mg under fed conditions (light, medium or fed), median tₘₐₓ occurred several hours earlier at 3 to 4 h post-dose. After both single and multiple dosing, Cₘₐₓ and AUC increased with increasing dose in a dose-proportional manner across the dose range studied. At a single-dose dose of 320 mg, Cₘₐₓ values increased from 1.4 to 1.7-fold when tonabersat was administered under fed conditions (medium or high-fat) compared to fasted, whereas AUC_{inf} was similar under fasted and fed conditions (285000 and 296000 h·ng/mL for 320 mg fasted and 320 mg fed, respectively). Therefore, total exposure (extent of absorption) was not affected by food administration, whereas the rate of drug absorption was increased in the presence of food. After single dosing with tonabersat, the geometric mean t_{1/2} was between 28.9 h and 37.8 h. The t_{1/2} was slightly prolonged after multiple dosing to a geometric mean t_{1/2} between 37.5 h and 41.6 h on Day 14. The half-life was not affected by fed conditions. After multiple dosing for 13 (DDI part) to 14 (MAD part) consecutive days, Cₘₐₓ and AUC for tonabersat accumulated approximately 3-fold (mean accumulation ratio AUC₀₋₂₄ ranged between 2.69 and 3.45 across the dose range tested), on Days 13 or 14 compared to Day 1 consistent with linear PK. Time to steady state following multiple dosing did not significantly differ between the ranges of doses tested. After multiple dosing with 240 mg, 320 mg or 400 mg tonabersat, steady state appeared to be reached after 7 to 9 days for all doses. These data may be used to determine dose and dose regimens to use in maintaining a steady state or the presence of other desired amounts of connexin hemichannel modulator (e.g., tonabersat) over a desired period of time.

In some embodiments, about 80 mg of a modulator is administered at least once per day, or BID, TID, or QID. In some embodiments, about 50 to about 80 mg of a modulator is administered at least once per day, or BID, TID, or QID. In some embodiments, about 75 to about 100 mg of a modulator is administered at least once per day, or BID, TID, or QID. In some embodiments, about 85 mg to about 125 mg of a modulator is administered at least once per day, or BID, TID, or QID. In some embodiments, about 125 mg to about 200 mg of a modulator is administered at least once per day, or BID, TID, or QID. In one embodiment, tonabersat, or another compound according to Formula I or Formula II, may be administered orally once or more per day at a dose of approximately 20 mg to approximately 40 mg. In one embodiment, tonabersat, or another compound according to Formula I or Formula II, may be administered orally once or more per day at a dose of approximately 40 mg to approximately 80, 100, 120, or 160 mg. In one embodiment, tonabersat, or another compound according to Formula I or Formula II, may be administered orally once or more per day at a dose of approximately 80 mg to approximately 100 mg. In one embodiment, tonabersat, or another compound according to Formula I or Formula II, may be administered orally once or more per day at a dose of approximately 100 mg to approximately 200 mg, including 120 and 160 mg. In one embodiment, tonabersat, or another compound according to Formula I or Formula II, may be administered orally once or more per day at a dose of approximately 200 mg to approximately 300 mg. In one embodiment, tonabersat, or another compound according to Formula I or Formula II, may be administered orally once or more per day at a dose of approximately 300 mg to approximately 400 or 500 mg. In some embodiments, the doses described herein, including milligram doses, are used for the oral administration of bioavailable modulator compounds. In some embodiments, these doses may be reduced for administration by injection (e.g., by 10-50% or more) or increased for topical administration (e.g., 50% to 100%). In any of these embodiments the modulating agent may be an organic compound modulator, a peptidomimetic modulator, or an antisense oligonucleotide modulator, for example. The above-noted dose concentrations may be decreased or increased as appropriate based on potency and specificity of the modulator, for example, and the nature or amount of a desired effect.

In some embodiments, the modulator may be administered in doses measured in milligrams per kilogram (i.e., at a specified number of milligrams per kilogram of weight of a subject (mg/kg) to which a modulator is being administered). In some embodiments, the average kg weight for a woman is from about 50 to about 80 kg. In some embodiments, the average kg weight for a man is from about 70 to about 90 kg. In some embodiments, the modulator may be administered at a dose between about 1.0 to about 3, 4 or 5 mg/kg, or any range between any two recited dosages or any dose between any two recited dosages. In some embodiments, these modulator doses may be administered in single dose per day, or in divided doses. In some embodiments, these modulator doses may be administered more than once per day (e.g., BID, TID, QID, etc.).

In some embodiments, a unit dose of a modulator, may be administered once or more than once a day in single or divided doses (for example 1, 2, 3, 4, 5 or 6, typically 1 to 4 times a day), such that the total daily dose is in the range (calculated for a 70 kg adult, with other amounts for subjects of different kg weights being adjusted as appropriate) of about 25 to about 400 mg, for example approximately 40 to approximately 80 mg, or about 80 mg to about 100 mg, about 100 to about 160 or 200 mg, or about 160 or 200 mg to about 300, 400 or 500 mg, or any amount or range between any two recited dosages. For example, a connexin hemichannel modulator, inflammasome modulator or pannexin channel modulator, may be administered to a subject at a dose range, for example, of approximately 0.5 to approximately 2, 3, 4 or 5 mg/kg/day, approximately 5 to approximately 10 mg/kg/day, or any range between any two recited dosages or any dose between any two recited dosages. In one embodiment, tonabersat may be administered orally once a day at a dose of approximately 1 mg to approximately 4 mg/kg/day.

Examples of effective doses that may be used as described herein may also be described in microgram per milliliter (μg/mL) amounts, or milligram per milliliter (mg/ml) amounts. In some embodiments, for the treatment of disease, disorders, defects or conditions described or referenced herein, a concentration of about 0.1 to about 10.0 microgram/ml, or from about 0.5 mg/mL to about 100 mg/mL, or more, or any range between any two of the recited dosages or any dose between any two recited numbers. The dose can be 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml or any range between any two of the recited dosages or any dose between any two recited numbers. In some embodiments, the therapeutically effective amount of the modulator, e.g. connexin and/or pannexin channel modulating agent protein modulating agent is present at a concentration ranging from about 0.5 to about 50 mg/mL. In some embodiments, the modulating agent is present at a concentration ranging from about 0.3 to about 30 mg/mL. In some embodiments, the modulating agent is present at a concentration ranging from about 0.1 or 1.0 to about 10 mg/mL. In some embodiments, the modulating agent is present at a concentration ranging from about 0.1 or 1.0 to about 0.3 or 3.0 mg/mL. In some embodiments, the modulating agent is present at a concentration of about 3.0 mg/mL. In any of these embodiments the modulating agent may be an organic compound modulator, a peptidomimetic modulator, or an antisense oligonucleotide modulator, for example. The above-noted dose concentrations may be decreased or increased as appropriate based on potency and specificity of the modulator, for example, and the nature or amount of a desired effect.

In one embodiment, the dose of compounds of formula I, for example tonabersat, and analogs of any of the foregoing compounds is approximately 0.1 micromolar and up to approximately 200 micromolar at or around a site of action, or higher, within the circulation to achieve those concentrations at the site of action. By way of example, the dose may be (but not limited to) a final circulating concentration or a concentration at or around a site of action of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 micromolar, or any range between any two recited concentrations, or any concentration between any two recited numbers. In some embodiments, tonabersat and other organic compounds (e.g., a compound according to Formula I, Formula II or Formula III) may be used at a lower dose, for example, 0.001 to 20 micromolar. A low dose can be 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, or 20 micromolar. In some embodiments, the dose of a modulator, e.g. a gap junction channel modulator is approximately 20 micromolar and up to approximately 200 micromolar, or 200 to 2000 or 5000 micromolar at the site of action, or higher within the circulation to achieve those concentrations at the site of action. By way of example, the dose may be (but not limited to) a final circulating concentration of about 1, 5, 10, 20, 50, 100, 200, 250, 500, 1000, 2000, 3000, 4000, or 5000 micromolar, or any range between any two recited dosages or any dose between any two recited dosages. In some embodiments, tonabersat, carabersat, probenecid and other organic compound modulators may be used at a lower dose, for example, 1 to 20 micromolar, 1 to 50 micromolar, 20 to 30, 30 to 40 or 40 to 50 micromolar.

All descriptions with respect to dosing, unless otherwise expressly stated, apply to the modulators of the invention, including connexin hemichannel modulators (e.g., connexin 43 hemichannel modulators), inflammasome modulators (e.g. NLRP3 inflammasome modulators) and pannexin modulators (e.g., pannexin 1 modulators). All descriptions with respect to dosing, unless otherwise expressly stated, also apply to all indications described or referenced herein. All descriptions with respect to dosing, unless otherwise expressly stated, apply to the therapeutic or prophylactic treatment of one or more signs and/or symptoms of a post-acute infection syndrome (e.g., long COVID, long flu, post-treatment Lyme disease syndrome, etc.). In some embodiments a dose or doses may be adjusted based on whether the modulator to be administered is a small molecule, a peptide or an oligonucleotide, for example, using knowledge of their half-lives and PK data.

### Formulations

Pharmaceutical compositions of the invention include various delivery forms and formulations, as desired or appropriate, including, for example, formulations for oral administration, parenteral administration, local administration, and topical administration of one or more modulators, as well as forms and formulations for modulator administration that are appropriate for other forms of administration, including those described or referenced herein, including matrices, implants, liposomes, microparticles, nanoparticles, solutions, gels and drops. Connexin hemichannel modulators (e.g., connexin 43 hemichannel modulators), inflammasome modulators (e.g., NLRP3 inflammasome modulators) and pannexin channel modulators (e.g., pannexin 1 channel modulators) may be formulated as compositions for any desired route of administration using methods and techniques in the art, whether now known or later developed.

Such delivery forms and formulations include those for the therapeutic and/or prophylactic treatment of a subject or a patient with a modulator or modulator composition as disclosed herein. In some embodiments, pharmaceutical formulations of the invention comprise at least one modulator and may further comprise one or more pharmaceutically acceptable excipients, diluents and/or carriers. Pharmaceutically acceptable diluents, carriers and/or excipients includes substances useful in preparing a pharmaceutical composition, and are generally safe, non-toxic and neither biologically nor otherwise undesirable. Pharmaceutically acceptable diluents, carriers and/or excipients include those suitable for veterinary use as well as human pharmaceutical use. By way of example, diluents, carriers and/or excipients include solutions, solvents, dispersion media, delay agents, polymeric and lipidic agents, emulsions and the like. By way of further example, suitable liquid carriers, especially for injectable solutions, include water, aqueous saline solution, aqueous dextrose solution, and the like, and vehicles such as liposomes being also especially suitable for administration of agents.

Suitable carriers and diluents include buffers, buffered solutions, aqueous solutions, saline, dextrose, glycerol, isotonic saline solutions, for example phosphate-buffered saline, isotonic water, and the like and combinations thereof. In some embodiments, carriers may include propylene glycol, dimethyl isosorbide, and water, and even more particularly, deionized water, monofunctional alcohols and symmetrical alcohols. In some embodiments, a pharmaceutically acceptable carrier or diluent may comprise or contain a thermosetting poloxamer (which may be a liquid or gel, depending on the temperature, e.g., a poloxamer), a carboxycellulose (e.g. carboxymethylcellulose), a collagen (e.g., a Type I collagen), a collagenous material comprising tropocollagen, a hyaluronan or derived-hyaluronic acid, and/or an oil (e.g., Emu oil). Suitable carriers can be large, slowly metabolized macromolecules such as proteins, polysaccharides, polylactic acids, polyglycolic acids, polymeric amino acids, and amino acid copolymers. In some embodiments, carriers used in formulations of the invention may include binders, buffering agents, coloring agents, diluents, disintegrants, emulsifiers, fillers, flavorings, glidants, lubricants, pH modifiers, preservatives, stabilizers, surfactants, solubilizers, tableting agents, and wetting agents. Some carriers may be in more than one class, for example vegetable oil may be used as a lubricant in some formulations and a diluent in others.

Other pharmaceutically acceptable carriers include sugars, starches, celluloses, powdered tragacanth, malt, gelatin, talc, and vegetable oils. Examples of other matrix materials, fillers, or diluents include lactose, mannitol, xylitol, microcrystalline cellulose, calcium diphosphate, and starch. Examples of surface-active agents include sodium lauryl sulfate and polysorbate 80. Examples of drug complexing agents or solubilizers include the polyethylene glycols, caffeine, xanthene, gentisic acid and cylodextrins. Examples of disintegrants include sodium starch glycolate, sodium alginate, carboxymethyl cellulose sodium, methyl cellulose, colloidal silicon dioxide, and croscarmellose sodium. Examples of binders include methyl cellulose, microcrystalline cellulose, starch, and gums such as guar gum, and tragacanth. Examples of lubricants include magnesium stearate and calcium stearate. Examples of pH modifiers include acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids. Optional other active agents may be included in a pharmaceutical composition, which do not substantially interfere with the activity of the compound of the present invention.

In some embodiments, the pharmaceutical composition for administration further includes an active modulator compound as described or referenced herein and optionally comprises one or more of a phosphoglyceride; phosphatidylcholine; dipalmitoyl phosphatidylcholine (DPPC); dioleoylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol such as polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxamer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl stearate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid; pullulan, cellulose, microcrystalline cellulose, hydroxymethylcellulose (HMC), hydroxypropyl methylcellulose (HPMC), hydroxyethylcellulose (HEC), hydroxypropyl cellulose (HPC), hydroxycellulose (HC), carboxymethylcellulose (CMC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan, mannitol, sorbitol, xylitol, erythritol, maltitol, and lactitol, a pluronic polymer, polyethylene, polycarbonate (e.g. poly(1,3-dioxan-2one)), polyanhydride (e.g. poly(sebacic anhydride)), polypropylfumerate, polyamide (e.g. polycaprolactam), polyacetal, polyether, polyester (e.g., polylactide, polyglycolide, polylactide-co-glycolide, polycaprolactone, polyhydroxyacid (e.g. poly((β-hydroxyalkanoate))), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-α-Tocopheryl polyethylene glycol 1000 succinate), gelatin, titanium dioxide, polyvinylpyrrolidone (PVP), block copolymers of ethylene oxide and propylene oxide (PEO/PPO), polyethyleneglycol (PEG), sodium carboxymethylcellulose (NaCMC), hydroxypropylmethyl cellulose acetate succinate (HPMCAS).

In some embodiments, the pharmaceutical preparation may include a polymer for controlled delivery of the described compounds, including, but not limited to, a pluronic polymer, polyester (e.g., polylactic acid, poly(lactic-co-glycolic acid), polycaprolactone, polyvalerolactone, poly(1,3-dioxan-2one)); polyanhydride (e.g., poly(sebacic anhydride)); polyether (e.g., polyethylene glycol); polyurethane; polymethacrylate; polyacrylate; and polycyanoacrylate. In some embodiments, the polymer may be modified with polyethylene glycol (PEG), with a carbohydrate, and/or with an acyclic polyacetal derived from a polysaccharide. See, e.g., Papisov, 2001, ACS Symposium Series, 786:301.

Compositions may be formulated for any desired form or route of delivery, including topical, instillation, parenteral, intramuscular, subcutaneous, transdermal administration, etc. In some embodiments, one more modulators of the invention, e.g., one or more connexin hemichannel modulators and/or inflammasome modulators, etc., are combined with a pharmaceutically acceptable carrier or diluent to produce a pharmaceutical composition or formulation. Modulators, e.g., one or more connexin hemichannel modulators and/or inflammasome modulators, etc., may be formulated as compositions for any desired route of administration, including topical, oral, systemic, transdermal, nasal, sublingual, buccal, etc. including formulations for injection (e.g. intracameral injection, subcutaneous injection, intramuscular injection, intravenous injection, etc.). In some embodiments, formulations of the invention are prepared for oral administration. In some embodiments, formulations of the invention are prepared for administered by injection (e.g. intravenous (IV), subcutaneous (SC/SQ), intraperitoneal (IP), intradermal (ID), or intramuscular (IM)). In some embodiments, the formulations of the invention are prepared for topical or local administration to a subject or a patient (e.g., to the skin or the eye). In some embodiments, the doses referenced herein of the invention are prepared for administration by other routes known in the art. In some embodiments, compounds disclosed or referenced herein are formulated for administration via implant, via tissue or organ implant (e.g., hepatic, renal, cerebral, ocular, heart, CNS, etc.), for local administration including for administration via a patch or in a matrix or bandage, for administration by inhalation or spray, for sublingual administration, for transdermal administration, for buccal administration, for rectal administration, for nasal or transnasal administration, for ocular administration (e.g. as an ophthalmic solution, gel or drop), or for intra-aortal, intracranial, subdermal, intraperitoneal, subcutaneous, sublingual, intrathecal administration, in dosage unit formulations containing conventional or other pharmaceutically acceptable carriers now known or later developed. Other useful formulations include slow or delayed release preparations. Slow and delayed release preparations may be prepared using established methods and techniques well known in the art.

Pharmaceutically acceptable salts can also be present, e.g., mineral acid salts such as hydrochlorides, hydrobromides, phosphates, sulfates, and the like; and the salts of organic acids such as citrates, acetates, propionates, malonates, benzoates, and the like.

In addition, if desired substances such emulsifying agents, stabilizing agents, or preservatives may also be present. In some embodiments, the pharmaceutical compositions of this invention for use in or on a subject or patient may comprise pH stabilizing buffers such as acetate buffers, citrate buffers, phosphate buffers, borate buffers and mixtures thereof. In some embodiments, the buffers useful in the present invention include boric acid, sodium borate, sodium phosphates, including mono, di- and tri-basic phosphates, such as sodium phosphate monobasic monohydrate and sodium phosphate dibasic heptahydrate, and mixtures thereof. In some embodiments, the preservative may be stabilized chlorine dioxide, cationic polymers or quaternary ammonium compounds. In some embodiments the pharmaceutical compositions may also comprise wetting agents, nutrients, viscosity builders, antioxidants, and the like, for example, disodium ethylene diamine tetraacetate, alkali metal hexametaphosphate, citric acid, sodium citrate, sodium metabisulfite, sodium thiosulfate, N-acetylcysteine, butylated hydroxyanisole, butylated hydroxytoluene, polyvinyl alcohol, polyoxamers, polyvinyl pyrrollidone, and mixtures thereof and mixtures thereof. In some embodiments, the pharmaceutical formulations of this invention will not include a preservative. In some embodiments, the connexin modulator composition or formulation comprises sodium phosphate dibasic heptahydrate or potassium phosphate, monobasic or both.

Where the modulator, e.g. connexin hemichannel modulator or inflammasome modulator is a nucleic acid, such as a polynucleotide, uptake of nucleic acids by mammalian cells may be enhanced by the of known transfection techniques including the use of transfection agents. Such techniques may be used with certain anti-connexin agents, including polynucleotides. The formulation that is administered may contain such transfection agents. Examples of useful transfection agents include cationic agents (for example calcium phosphate and DEAE-dextran) and lipofectants (for example Lipofectam™ and Transfectam™), and surfactants.

Pharmaceutically acceptable excipients are known in the art, including to those of ordinary skill in the art. In some embodiments, the formulations may provide for sustained delivery of a modulator a subject, a patient, or to a selected tissue, organ, segment or compartment of a subject or a patient. In some embodiments, the formulations provide high (including via topical or oral administration, for example) drug bioavailability, are safe and non-toxic, and/or have little systemic side effects or complications at a site of administration. In some embodiments, modulator formulations for use in the methods of this invention have the ease of localized delivery and the ease of oral administration. In particular, connexin hemichannel modulator formulations for use in the methods of this invention also have the benefits of a “no side effect” profile.

In some embodiments the pharmaceutical formulations of this invention may comprise any of the modulators described or referenced herein, e.g. connexin hemichannel modulators, inflammasome modulators and/or pannexin channel modulators described herein. In some embodiments, the pharmaceutical compositions of the invention can include or exclude any of the foregoing modulator classes. In some embodiments, the pharmaceutical compositions of the invention can include or exclude any connexin hemichannel modulator known in the art (including, e.g., any known connexin 43 hemichannel modulator). In some embodiments, the pharmaceutical compositions of the invention can include or exclude any inflammasome modulator known in the art (e.g., any known NLRP3 channel modulator). In some embodiments, the pharmaceutical compositions of the invention can include or exclude any pannexin channel modulator known in the art (e.g., any known pannexin 1 channel modulator).

Any of the modulators described or referenced herein, e.g. connexin hemichannel modulators, inflammasome modulators and/or pannexin channel modulators may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 75%, about 80%, about 85%, or about 90%, e.g. at least about 88%, at least about 90%, 95% or 98%, or at least about 99% of a modulator or dry mass of the preparation.

A pharmaceutical composition may be formulated as any pharmaceutically useful form or dosage form, e.g., including tablets, pills, capsules, gel caps, semisolids, powders, syrups, sustained release formulations, solutions, suspensions, elixirs, aerosols, inhalation formulations, liquids for injection and/or infusion, lyophilized compounds and compositions, gels, creams, microparticles, nanoparticles transdermal delivery devices (for example, a transdermal patch), subcutaneous delivery devices (for example, a subcutaneous patch), inserts and implants, in or on a medical device or implant, or any other appropriate compositions, including suppository, buccal, or sublingual formulations. Some dosage forms, such as tablets and capsules, are subdivided into suitably sized unit doses containing appropriate quantities of the active components, e.g., an effective amount to achieve the desired purpose. Compositions may take the form of any standard known dosage form. Persons of ordinary skill in the art to which the invention relates will readily appreciate the most appropriate dosage form having regard to the nature of the condition to be treated and the active agent to be used (e.g., antisense, peptidomimetic, small molecule, etc.) without any undue experimentation.

Pharmaceutical compositions, and methods of manufacturing such compositions, suitable for administration as contemplated herein are known in the art. Examples of known techniques include, for example, U.S. Pat. Nos. 4,983,593, 5,013,557, 5,456,923, 5,576,025, 5,723,269, 5,858,411, 6,254,889, 6,303,148, 6,395,302, 6,497,903, 7,060,296, 7,078,057, 7,404,828, 8,202,912, 8,257,741, 8,263,128, 8,337,899, 8,431,159, 9,028,870, 9,060,938, 9,211,261, 9,265,731, 9,358,478, 9,387,252, and others.

In some embodiments, the compounds of the present invention can be formulated as particles. In one embodiment the particles are or include microparticles. In one embodiment the particles are or include nanoparticles. Common techniques for preparing particles include solvent evaporation, solvent removal, spray drying, phase inversion, coacervation, and low temperature casting. Pharmaceutically acceptable excipients, including pH modifying agents, disintegrants, preservatives, and antioxidants, can optionally be incorporated into particles during particle formation. In one embodiment, the particles are derived through a solvent evaporation method. In this method, a compound described herein (or polymer matrix and one or more compounds described herein) is dissolved in a volatile organic solvent, such as methylene chloride. The organic solution containing a compound described herein is then suspended in an aqueous solution that contains a surface active agent such as poly(vinyl alcohol). The resulting emulsion is stirred until most of the organic solvent evaporated, leaving solid nanoparticles or microparticles. The resulting nanoparticles or microparticles are washed with water and dried overnight in a lyophilizer. Nanoparticles with different sizes and morphologies can be obtained by this method.

In some embodiments of the invention, a nanoparticle or microparticle comprises poly(lactic-co-glycolic acid) (“PLGA”) loaded with one or more modulators, for example, connexin 43 modulators and/or NLRP3 inflammasome modulators. The modulators can be loaded into the particle volume, onto the particle exterior surface, or both. In some embodiments, the particle formulations of any of the modulators of this disclosure (e.g. connexin 43 modulators, NLRP3 inflammasome modulators, pannexin 1 channel modulators) may also comprise liposomes.

In some embodiments of the invention, modulators may also be formulated to provide controlled release to a cell, group of cells, organ, subject, or patient. In some embodiments of this invention, the formulations may be immediate, or extended or sustained release dosage forms, e.g., for release within several hours, within one day or, for example, within 1-2 days.

Compositions may be formulated in accordance with standard methods and techniques known in the art. Techniques and methods include those found in such standard references as Gennaro A R: Remington: The Science and Practice of Pharmacy, 20^{th} ed., Lippincott, Williams & Wilkins, 2000, for example.

Any container suitable for storing and/or administering a pharmaceutical composition may be used in a product or combination product of the invention. Suitable containers will be known and appreciated by persons skilled in the art. Containers may include vials and syringes. The containers may be suitably sterilized and hermetically sealed.

In some embodiments, administering a modulator (for example, a connexin 43 modulator or connexin 45 modulator, an NLRP3 inflammasome modulator, or a pannexin 1 channel modulator), to a subject or patient provides a therapeutically effective amount of the modulator to the subject or patient or a specific organ, compartment or other target part of the subject or patient by means of administration as desired and appropriate depending on the nature of the connexin modulator, including but not limited to oral administration, enteral administration, parenteral administration, topical administration, intramuscular administration, intravenous administration, etc.

Therapeutically effective amounts include but are not limited to the doses described herein. Described doses and other therapeutically effective amounts are administered in one or more of the therapeutically effective dose regimens described herein.

### Administration

Administration of a modulator, e.g. connexin hemichannel modulator, inflammasome modulator, pannexin channel modulator compounds and compositions may be administered by one of the following routes: oral, topical, systemic (including intravenous, intra-arterial, intra-peritoneal, transdermal, intranasal, or by suppository), parenteral (including intramuscular, subcutaneous, or intravenous or intra-arterial injection), and the like. In some embodiments, connexin hemichannel modulator, inflammasome modulator, and pannexin channel modulator compounds and compositions are administered orally. Bioavailable systemically administered modulators may be administered, with or without concomitant local administration to a target organ, for example, by oral administration. In some embodiments, connexin hemichannel modulator, inflammasome modulator, and pannexin channel modulator compounds and compositions are administered transdermally or by inhalation. In some embodiments, connexin modulator, gap junction channel modulator and/or hemichannel modulator compounds and compositions are administered topically (e.g., to the skin or eye). In some embodiments, the connexin modulator, gap junction channel modulator and/or hemichannel modulator compounds and compositions are administered by injection.

Topical formulations of the gap junction, hemichannel and/or connexin modulators can comprise ointments, gels, which may be, for example, thermosetting gels, drops, sprays, liquids and powders, or a sustained or non-sustained release dosage form.

In some embodiments, the connexin modulator is embedded in a matrix comprising a bandage or other implantable device. In some embodiments, the matrix provides for slow or sustained release of the connexin hemichannel modulator, inflammasome modulator, and/or pannexin channel modulator. As used herein, “matrix” includes for example, matrices such as polymeric matrices, biodegradable or non-biodegradable matrices, and other carriers useful for making implants or applied structures for delivering one or more modulators. Compositions and methods for the preparation of biodegradable or non-biodegradable matrices have been developed and are known in the art.

The pharmaceutical composition may be formulated as any pharmaceutically useful form, e.g., as an aerosol, a cream, a gel, a gel cap, a pill, a microparticle, a nanoparticle, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution or suspension. Some dosage forms, such as tablets and capsules, are subdivided into suitably sized unit doses containing appropriate quantities of the active components, e.g., an effective amount to achieve the desired purpose.

In some embodiments, the pharmaceutical compositions and formulations of this invention can exclude a connexin hemichannel modulator (a connexin 43 hemichannel modulator), an inflammasome modulator (e.g., an NLRP3 inflammasome modulator) and/or a pannexin channel modulator (e.g., a pannexin 1 channel modulator) in sterile water as the only vehicle. In some embodiments, the pharmaceutical compositions and formulations of this invention can exclude a connexin hemichannel modulator (a connexin 43 hemichannel modulator), an inflammasome modulator (e.g., an NLRP3 inflammasome modulator) and/or a pannexin channel modulator (e.g., a pannexin 1 channel modulator) in a vehicle used in cell or animal research that is not approved for human use. In some embodiments, the pharmaceutical compositions and formulations of this invention can exclude any other vehicle used or known the art.

### Articles of Manufacture/Kits

In some embodiments of the invention, an article of manufacture, or “kit”, containing materials useful for treating or preventing a disease, defect, disorder or condition described or referenced herein is provided. In some embodiments of the invention, an article of manufacture, or “kit”, contains materials useful for treating and/or preventing one or more signs and/or symptoms (e.g., fatigue, brain fog, etc.) of a post-infection syndrome (e.g., long COVID, long flu, post-treatment Lyme disease syndrome, etc.).

In some embodiments, the kit comprises a therapeutically or prophylactically effective amount of a connexin hemichannel modulator, an inflammasome modulator and/or a pannexin channel modulator. In some embodiments, the kit includes at least one connexin hemichannel modulator. In some embodiments, the kit includes at least one connexin 43 hemichannel modulator. In some embodiments, the kit includes at least one inflammasome modulator that is a direct inflammasome modulator. In some embodiments, the kit includes at least one inflammasome modulator that is an indirect inflammasome modulator. In some embodiments, the kit includes at least one direct and/or indirect NLRP3 inflammasome modulator. In some embodiments, the kit includes at least one pannexin channel modulator. In some embodiments, the kit includes at least one pannexin 1 channel modulator. In some embodiments, the kit includes one or more connexin hemichannel modulators, one or more direct and/or indirect inflammasome modulators, and/or one or more pannexin channel modulators.

In some embodiments, the kit includes a pharmaceutical composition comprising one or more therapeutically or prophylactically effective doses of a connexin hemichannel modulator (e.g., a connexin 43 hemichannel modulator). In some embodiments, the kit includes a pharmaceutical composition comprising one or more therapeutically or prophylactically effective doses of a direct and/or indirect inflammasome modulator (e.g., a direct and/or indirect NLRP3 inflammasome modulator). In some embodiments, the kit includes a pharmaceutical composition comprising one or more therapeutically or prophylactically effective doses of a pannexin channel modulator (e.g., a pannexin 1 channel modulator). In some embodiments, the inflammasome modulator being dosed is a peptidomimetic pannexin channel modulator (e.g., 10Panx). In some embodiments, the pannexin channel modulator being dosed is an antisense pannexin channel modulator.

In some embodiments, the kit comprises one or more pharmaceutical compositions, in separate vessels, or a partitioned vessel, together with packaging and instructions for use. In some embodiments, the kit may also comprise a pharmaceutically acceptable carrier. In some embodiments the kit may also include components for administering a modulator or modulator composition, for example, a syringe, needle, microneedle, etc.

In some embodiments, the kit includes one or more containers. In some embodiments, one or more containers in a kit comprise one or more connexin hemichannel modulators, one or more direct and/or indirect inflammasome modulators, and/or one or more pannexin channel modulators. Suitable containers include, e.g., bottles, vials, etc. The container may be formed from any of a variety of known useful materials, for example, glass or plastic.

In some embodiments, the kit includes a label or a package insert or both, on or associated with the container, which includes instructions for dosing. The term “package insert” refers to instructions customarily included in commercial packages of pharmaceutical and research products that contain information about the indications, usage, dosage, administration, contraindications and/or warnings concerning the use of a modulator, which may be provided physically or via an online link, for example, such as a QR code or the like. In some embodiments, the label or package insert indicates that the composition is used for treating or preventing one or more signs and/or symptoms (e.g., fatigue, brain fog, etc.) of a post-infection syndrome (e.g., long COVID, long flu, post-treatment Lyme disease syndrome, etc.). In some embodiments, the kit, label, package, and/or package insert comprises information on the dosing of one or more modulators on separate days and may include packaging and/or instructions for chronologically or daily staggered administration in accordance with methods of the invention.

In some embodiments, kits are manufactured for in vivo use, e.g., for delivery of modulator compounds or compositions to a subject or patient. In some embodiments, kits are manufactured for in vitro use, e.g., for delivery of modulator compounds or compositions to a cell or cell culture. In some embodiments, kits are manufactured for ex vivo use, e.g., for delivery of modulator compounds or compositions to a tissue or organ.

### Manufacture

The chemical modulators, peptidomimetic modulators, and antisense modulators of the invention may be purchased from available manufacturers, or manufactured using chemistries known in the art for synthesizing organic compounds (e.g. tonabersat, and other compounds of Formula I and Formula II), chemistries known in the art for synthesizing and preparing peptides and peptidomimetics, and solid-phase and other chemistries and methods known in the art for synthesizing oligonucleotides. In in some embodiments, a formulation of the invention will comprise a salt of a chemical modulator, peptidomimetic modulator, or an antisense modulator.

In some embodiments, the formulations of this invention are substantially pure. By substantially pure is meant that the formulations comprise less than about 10%, 5%, or 1%, and preferably less than about 0.1%, of any modulator or non-modulator impurity. In some embodiments the total impurities, including metabolites of a connexin modulator, inflammasome modulator, or pannexin channel modulator, will be not more than 15%. In some embodiments the total impurities, including metabolites of the connexin 43 modulating agent, will be not more than 12%. In some embodiments the total impurities, including metabolites of a connexin modulator, inflammasome modulator, or pannexin channel modulator, will be not more than 11%. In other embodiments the total impurities, including metabolites of a connexin modulator, inflammasome modulator, or pannexin channel modulator, will be not more than 10%.

Sterile compositions comprising a connexin modulator, inflammasome modulator, or pannexin channel modulator of the invention may be prepared using aseptic processing by dissolving a connexin modulator, inflammasome modulator, and/or pannexin channel modulator in the formulation vehicle. In some embodiments, the formulation is be sterilized, or further sterilized, by filtration. Excipients used in the manufacture of formulations of the invention are widely used in pharmaceutical products and released to pharmacopeial standards.

### Methods for Quantifying Modulator Activity

In another aspect the present disclosure provides methods of evaluating, comparing, selecting, or quantifying the activity of, or determining the presence or amount of a modulator compound or composition or candidate modulator compound or composition for use as described herein.

In some embodiments, the method comprises the steps of (1) providing a first isolated cell or preparation of cells; (2) determining ATP levels, (a) inside and/or outside a isolated cell or preparation of cells, and/or (b) within the cell or cell preparation; (3) optionally, comparing the ATP levels in the cell or cell preparation to a reference ATP level; (4) contacting the cell or cell preparation with a test compound (e.g., one or more connexin hemichannel or pannexin channel modulators or candidate modulators), and/or contacting the test compound with another preparation of like or similar cells, and (5) remeasuring ATP in the cell or preparation of cells. In some embodiments, the results following remeasuring are compared to one or more or all of (i) a positive control run using the same method, (i) a negative control run using the same method, (iii) a historic positive control, (iv) a historic negative control, (v) a reference modulator compound, and/or (vi) a reference compound known to alter the release (positively or negatively) of ATP through a connexin hemichannel (e.g., a connexin 43 hemichannel) or a pannexin channel (e.g. a Panx1 channel). In some embodiments, ATP levels are measured using a method known in the art. In some embodiments, the positive connexin hemichannel modulator control is tonabersat. In some embodiments, results the negative control is saline or another vehicle.

In some embodiments, a test composition comprising more than one test compounds is evaluated in the method. Positive test compositions are reduced into a smaller number of test compounds and run again. The method is carried out until one or more positive test compounds in the positive test composition is/are identified.

In some embodiments of these methods, the isolated cell or preparation is/are retinal pigment epithelial cell(s) (e.g., ARPE-19 cell(s)). In some embodiments of these methods, the isolated cell or preparation is/are brain cell(s). In some embodiments, the “cell or preparation of cells” used in the method is a tissue. In some embodiments, the tissue is brain tissue. In some embodiments, the cells, cell preparation or tissue are human or of human origin.

In some embodiments, methods of evaluating, comparing, selecting, or quantifying the activity of, or determining the presence or amount of a modulator compound or composition or candidate modulator compound or composition for use as described herein are carried out in vitro. In other embodiments, the methods are carried out on a chip (e.g., an “organ on a chip”). In some embodiments, the test method is automated.

In some embodiments, the test compound is a connexin hemichannel modulator or a candidate connexin hemichannel modulator.

In some embodiments, the test compound is an inflammasome modulator or a candidate inflammasome modulator. In some embodiments, the test compound is an NLRP3 inflammasome modulator or candidate NLRP3 inflammasome modulator. In some embodiments, the test or candidate inflammasome modulator is a connexin hemichannel modulator or a candidate connexin hemichannel modulator (e.g. a connexin 43 hemichannel modulator or a candidate connexin 43 hemichannel modulator). In some embodiments, the test or candidate inflammasome modulator is a pannexin channel modulator or a candidate pannexin channel modulator (e.g. a pannexin 1 hemichannel modulator or a candidate pannexin 1 hemichannel modulator).

In some embodiments, the test compound is a small molecule that modulates, may modulate, or is suspected of modulating a connexin hemichannel (e.g., a connexin 43 hemichannel). In some embodiments, the test compound is a small molecule that modulates, may modulate, or is suspected of modulating an inflammasome or inflammasome activity (e.g., an NLRP3 inflammasome inhibitor or NLRP3 inflammasome activity). In some embodiments, the test compound may be a small molecule that modulates, may modulate, or is suspected of a pannexin channel (e.g., a pannexin 1 channel). In some embodiments, the test compound is a peptidomimetic. In some embodiments, the test compound is an antisense compound.

In some embodiments of this test/screening method, tonabersat, Peptide5 or XG19 is used as a positive control.

### EXAMPLES

The below experiments relate to the inventions described herein, including the use of connexin hemichannel modulators (e.g. connexin 43 connexin hemichannel modulators), inflammasome modulators (e.g., NLRP3 inflammasome modulators) and pannexin channel modulators (e.g. pannexin 1 hemichannel modulators), including for the therapeutic and/or prophylactic treatment of one or more signs and/or symptoms of a post-acute infection syndrome, e.g., long COVID.

### Example 1

Phase 2 Study to Compare the Efficacy and Safety of an Orally Administered Connexin Hemichannel Modulator with Placebo in Patients with Ocular and Systemic Manifestations of Post Covid Sequelae Known as “Long Covid”

This Example describes the use of connexin hemichannel modulators and inflammasome modulators to treat and prevent one or more signs and/or symptoms of long COVID. The protocol will enroll 15 subjects at a single site in the United States. The objective of is to assess the safety and efficacy of once daily orally administered Xiflam™ (80 mg), a connexin hemichannel and inflammasome modulator, compared to a Placebo control in subjects with long COVID.

This is an investigational, randomized, masked, placebo-controlled study, designed to evaluate the ocular and systemic efficacy and safety of Xiflam™ (80 mg QD tonabersat) in subjects with long COVID over a 12-week period.

Subjects will be evaluated for one, two, three, four, five, six, seven, eight, nine, or ten of the following symptoms: (1) Brain/Neurological symptoms, including brain fog, peripheral neuropathies (pins & needles/numbness any body part), dizziness, confusion, hallucinations, loss of smell and/or taste, phantom smells and/or taste, tinnitus (ringing in ears), internal vibrations/tremors, speech issues/stutter, stroke, seizures, and headaches; (2) Ocular symptoms, including eye pain, burning eye pain, feeling of sand/glass in eyes, eye redness, vision changes, visual disturbances/floaters; (3) Breathing/Respiratory symptoms, including shortness of breath, cough, post exertional malaise (tired after minor exertion), fatigue or extreme fatigue/exhaustion without exertion; (4) Digestive/Gastrointestinal symptoms, including nausea, vomiting, diarrhea, bloating, and early satiety (feeling full after eating small amounts of food); (5) Cardiovascular symptoms, including heart arrythmias (heart misses beats), tachycardia (rapid heartbeat), bradycardia (slow heart beat) and chest pain; (6) Musculoskeletal symptoms, including joint pain, muscle pain and bone pain; (7) Dermatological symptoms, including skin rashes and hair loss; (8) Mental health symptoms, including anxiety, and depression; (9) Miscellaneous symptoms, including flushing of face, facial paralysis, ptosis (drooping eyelid), bruising, cold hands/feet, heat/cold intolerance, feeling of fever without having one, and mottling (skin discoloration) of hands and/or feet; and (10) “Other” symptoms. On a 1-10 scale, long COVID symptoms will be designated as “none” (0), “mild’ (1-3), “moderate” (4-7) and “severe” (7-10).

The primary endpoint is the mean difference in ocular and systemic signs and symptoms of long COVID between the treated and placebo groups at 12 weeks, with an interim analysis at 6 weeks.

The secondary endpoint are (1) mean change in Best Corrected Visual Acuity BCVA form baseline to week 12; (2) mean change from baseline in anterior and posterior ocular signs and symptoms; (3) mean change from baseline in cardiac and neural signs and symptoms; and (4) mean change in laboratory values including inflammatory markers, e.g., CRP (C-reactive protein), ESR (erythrocyte sedimentation rate), ANA (Antinuclear Antibody), and others to be decided, from baseline to week 12.

The safety endpoint is the incidence and severity of ocular and systemic treatment emergent adverse events (TEAE).

The Investigation Product administered to subject is Xiflam™ (80 mg tonabersat) or Placebo control orally dosed once a day for 12 weeks. Two (2) treatment arms will be evaluated in this study. Randomization will be in a 2:1 ratio: GROUP 1 (n=10) Xiflam™ 80 mg QD and GROUP 2 (n=5) Placebo 0 mg QD.

Subjects who meet entry criteria for the study will be randomized to either Xiflam™ 80 mg or Placebo, all dosed orally once a day. The subjects, the Investigator, and all site personnel responsible for performing study assessments will be masked to treatment assignment for the duration of the study. Subjects must meet all inclusion/exclusion criteria. The study will consist of an initial qualification visit and if qualified, the patient will sign an informed consent. Once signed the patient will be randomized. Following randomization, subjects will be provided with IP and receive instruction regarding dosing and ongoing care. Patients will receive a follow-up call one week later. Thereafter study visits will occur in the clinic at 6 weeks and 12 weeks. An interim analysis will be carried out at 6 weeks.

Inclusion Criteria: A subject will be eligible for inclusion in this study only if ALL of the following criteria apply:
1. Have tested positive for SARS-CoV-2 (COVID-19) irrespective of variant or timeframe.
2. Developed signs and symptoms of the disease as described by the CDC and WHO.
3. Have recovered from the infection (tested negative for COVID-19).
4. Following negative COVID-19 testing, continued to demonstrate signs/symptoms which were not pre-existing. The signs/symptoms must have persisted for 12 weeks or more.
5. Have had a persistent recurrence of a disease state (e.g., posterior uveitis, extreme fatigue etc.) that occurred following COVID-19 infection.
6. Female subjects must be:
   a. Women of non-child-bearing potential, or
   b. Women of child-bearing potential with a negative pregnancy test at screening, must agree to use approved methods of contraception for the duration of the study and refrain from breastfeeding for the duration of the study.
7. Males with female partners of child-bearing potential must agree to use approved methods of contraception and agree to refrain from donating sperm for the duration of the study.
8. Willing and able to give informed consent and to comply with the study procedures and assessments.

Exclusion Criteria: A subject not will be eligible for inclusion in this study if any of the following exclusion criteria apply:
1. No proof of having tested positive for COVID-19 infection at any time.
2. Presence of an active ocular/systemic disease that in the opinion of the Investigator existed prior to COVID-19 infection and is not likely a LC related condition.
3. Intraocular surgery (including lens replacement surgery) within 3 months prior to randomization.
4. History of laser therapy in the macular region.
5. Any ocular or systemic condition that in the opinion of the Investigator is not LC related (e.g., pre-existing cataract) that may require surgery or medical intervention during the study period.
6. Participation in any systemic experimental treatment or any other systemic investigational new drug within 90 days prior to the start of study treatment.
7. Any screening laboratory value (hematology, serum chemistry or urinalysis) that in the opinion of the Investigator is clinically significant and not suitable for study participation.
8. Known hypersensitivity to Xiflam™ or excipients.
9. Known history of alcohol and/or drug abuse within 12 months prior to Visit 1/Screening that, in the opinion of the Investigator, may interfere with study compliance, outcome measures, safety parameters, and/or the general medical condition of the subject.

TABLE 1
Schedule of assessments

| | Procedure | | | | |
|---|---|---|---|---|---|
| | | Day 1 | | | |
| | Screening / | (within 1 week | Week 1 | | |
| | Qualification | of Screening if | (Telephone | | Week 12 |
| | Assessment | eligible) | Contact) | Week 6 | EXIT |
| | Week | | | | |
| | | | W1 | W6 | W12 |
| | Visit Windows | | | | |
| | | | +/−2 | +/−5 | +/−5 |
| Informed consent | X | | | | |
| Assessment of | X¹ | X¹ | | | |
| eligibility (Inc/Exc | | | | | |
| criteria). | | | | | |
| Demographics | X | | | | |
| Ocular/Medical | X | | | | |
| History, Concomitant | | | | | |
| meds | | | | | |
| Physical Exam | X | | | | X |
| Vitals | X | X | | X | X |
| Clinical Labs | X | | | | X |
| (Blood/Urine) | | | | | |
| Pregnancy Testing | X ² | X ² | | | X ² |
| Complete Ophthalmic | X | | | X | X |
| Exam³ | | | | | |
| NEI VFQ-25⁴ | X | X | | X | X |
| Randomization | | X | | | |
| IP dispensing | | X | | X | |
| IP accountability | | | | X | X |
| Assess adverse events ^{5, 6} | | X | X | X | X |

¹Must be done prior to randomization of Investigational Product.
² Only required for women of child-bearing potential. A negative result must be confirmed before first dose of Investigational Product can be administered.
³BCVA (ETDRS) Ophthalmoscopy, slit lamp examination, intraocular pressure and other testing that may be necessary in the opinion of the Investigator.
⁴National Eye Institute 25-item Visual Function Questionnaire (NEI VFQ-25) administered by Investigator.
⁵ Only adverse events (AE) that occur post Investigational Product at Day 1 are to be recorded on the Case Report Form (CRF) as TEAE.
⁶ Final AE assessment will take place 30 days after the last dose of IP.

Data Collection and Testing Criteria: The following procedures are needed to confirm eligibility and/or serve as baseline measures for the randomized trial:
If a procedure has been performed using the study technique and by study certified personnel as part of usual care, then it does not need to be repeated specifically for the study if it was performed within the defined time windows specified below.
The testing procedures; Visual acuity, ocular exam, OCT, blood draw, and serum sample will be performed by the Sponsor's certified personnel.
1. Self-reported demographics (date of birth, sex, race, and ethnicity).
2. Medical history of COVID-19 infection, pre-existing medical conditions, concomitant medications, as well as ocular diseases, surgeries, and treatment.
   Medical/Ocular history will be obtained by medical charts if available at the enrolling site; otherwise, it will be self-reported.
3. ETDRS visual acuity testing at 4 meters using the Electronic Visual Acuity Tester (including protocol refraction) in study eye only (within prior 14 days of randomization).
4. Ocular examination on each eye including slit lamp, IOP, lens assessment, and dilated ophthalmoscopy (within prior 14 days of randomization).
5. Measurement of blood pressure.
6. Fasting blood draw within 8 days of enrollment for:
   C-reactive protein (CRP), complete blood counts (CBC), comprehensive metabolic panel (CMP), erythrocyte sedimentation rate (ESR or sed rate). The central laboratory must be used—see study procedure manual for collection procedure.
   A small snack and drink will be available for the participant after blood draws are performed.
   Urine pregnancy test for all females who are premenopausal and are not surgically sterile will be performed (within 7 days of enrollment).

Screening procedures will last approximately 3 hours.

Randomization of Eligible Participants: Prior to randomization, the study participant's understanding of the trial, willingness to accept the assigned treatment group, and commitment to the follow-up schedule should be reconfirmed. The initial 6-week supply of randomized medication (Xiflam™ or placebo) will be provided on the day of randomization. The Xiflam™ and placebo tablets will be provided in masked bottles by the Coordinating Center, or designee, to the clinical sites for distribution to the study participants. A study participant should not be randomized without both types of medication available in inventory.

Study participants will be randomly assigned to one of the treatment groups: 80 mg Xiflam™ and Placebo. Each group will be asked to start daily supplementation. Two 40 mg tablets of study drug Xiflam™ or two tablets of placebo on the day of randomization.

Screen Failures: Individuals who do not initially meet study eligibility requirements may be rescreened at a later date per Investigator discretion.

### Randomized Trial Procedures:

Home Procedures—All participants will be instructed verbally regarding medication administration and be provided with an instruction sheet to take home. All participants will be provided a bottle of 40 mg Xiflam™ tablets and instructed to take 2 tablets per day preferably at night before retiring. The masked study drug may be taken with or without food (without being preferred). The initial 6-week supply will be provided by the site on the day of randomization. Thereafter, study drug supply will be provided at each subsequent follow-up visit. Details regarding study drug accountability are included in the coordinator manual.

Text Message Reminders: For participants who agree to receive text message reminders, periodic messages will be sent by the Coordinating Center with a reminder to comply with the study drug schedule.

Study Visits and Phone Contacts: A phone call is completed by site personnel within approximately one week of randomization (5 to 10 days) to emphasize compliance with study drug use and answer any questions. Additional phone contacts may be performed as needed.

The schedule of protocol-specified assessment visits is detailed below. If rescue treatment is deemed necessary during the active study period, the 12-week visit study procedures will be performed prior to initiating treatment. Once a patient has received alternative treatment, Xiflam™ will be discontinued and follow-up will continue until completion of the 12-week visit. Since there is no approved treatment for patients with ocular and systemic manifestations of post COVID sequelae known as LC, rescue would be at the discretion of the Investigator.

Study Visits: Study visits will occur as shown in Table 2.

TABLE 2
Study visits

| | Allowable Window |
|---|---|
| Target Day/Week | (Around Target Day/Week) |
| 1 week | ±2 days |
| 6 weeks | ±5 days |
| 12 weeks | ±5 days |

The goal will be for all participants to complete all scheduled visits. However, participants who (because of unforeseen circumstances) are unable or unwilling to return for all follow-up visits will be permitted to return for key visits only as an alternative to withdrawal from the study. When a participant is placed into this status, missed visits will not be recorded as protocol deviations (since they would not be recorded as protocol deviations if the participant was dropped from the study). Additional office visits may occur as needed.

Procedures at Study Visits: The following procedures will be performed in groups at each visit.
1. A review of any outstanding and collection of any new AEs.
2. Symptomatology at each visit measured by Visual Analog Scale (0-100).
3. ETDRS visual acuity testing (best corrected) in both eyes.
4. Ocular exam including slit lamp examination, lens assessment, measurement of intraocular pressure and dilated ophthalmoscopy (at Investigator discretion) on both eyes at every visit.
5. NEI VFQ-25
6. Urine sample for pregnancy test for all females who are premenopausal and are not surgically sterile.
7. Blood Pressure
8. Whole blood sample for CRP, CBC, CMP, and ESR collected at baseline, and then at 12 weeks.
9. Compliance assessment.

Compliance Assessment at Study Visits: At each visit the participant will receive a new supply of medication. The participant will be asked to bring their remaining pills to the clinic for compliance assessment. If a visit is missed, the participant may be sent a limited supply via mail and asked to send the remaining pills using a pre-addressed return package back to the site. Site personnel will count the remaining pills for each returned bottle and enter the number on an eCRF, which will calculate the compliance level.

Early Termination Visit: Study participants who request to withdraw will be asked to have a final closeout visit at which the testing described for study visits will be performed.

Unscheduled Visits: Additional visits may occur as required for usual care of the study participant. Testing procedures at unscheduled visits are at Investigator discretion.

Treatment Initiation Visit: Participants for whom alternative treatment is planned for the first time at an unscheduled visit will be asked to complete the study procedures above before the treatment is administered.

Participant Access to Study Agent at Study Closure: Any remaining study drug must be returned at the end of the study. Participants who withdraw from the study will be asked to return the remaining drug for a final compliance assessment.

Unmasking Procedure: In general, all study personnel and participants will remain masked to the treatment assignment throughout the duration of the study. This applies even if the participant's dose is discontinued. In certain circumstances, a site may request that the participant and/or care provider be unmasked to the treatment group assignment during the study if medically necessary. In general, unmasking is only permitted in cases where the Investigator deems it necessary for the safety or care of the participant. Approval results from consultation between the Investigator and the Medical Monitor.

Testing Procedures: The testing procedures are detailed in the Sponsor's procedures manuals. An overview of the equipment and certification requirements for all testing are shown in Table 3.

TABLE 3
Testing equipment and certification requirements

| | Equipment Required | |
|---|---|---|
| Study Procedures | (If applicable) | Who can Perform |
| Ocular Exam (including slit lamp | Any equipment is acceptable | Certified Investigator |
| examination, lens assessment, and | | |
| dilated ophthalmoscopy) | | |
| Physical examination (height and | Any equipment is acceptable | Certified Investigator or coordinator |
| weight) | | |
| Blood pressure | Proper size blood pressure cuff | Certified Investigator or coordinator |
| Intraocular pressure (IOP) | A Goldmann tonometer should be | Does not need to be performed by |
| | used if available. If not, same | study certified personnel* |
| | tonometer must be used for the | |
| | duration of the study | |
| ETDRS - BCVA | EVA refraction chart and trial frames | Clinical site personnel certified for |
| | | protocol refraction |
| NEI VFQ-25 | Validated Instrument | Clinical site personnel certified for |
| | | NEI VFQ-25 |
| Sample collection and processing | Centrifuge required; collection kit | Does not need to be performed by |
| | provided by study | study certified personnel* |
| Sample shipment | Shipping materials provided by | IATA trained personnel* |
| | study; dry ice required for biomarker | |
| | samples | |
| Pregnancy Urine Test | Urine collection and HCG testing kit | Clinical site personnel certified for |
| | provided by the study | Pregnancy Testing |

*Personnel who will be performing procedure must be documented in the Study Staff Deletion Log. The Principal Investigator (PI) is responsible for verifying individual qualifications and training specific to performing each type of procedure and ultimate accuracy and integrity of such data.

Patents, publications, scientific articles, web sites, and other documents and materials referenced or mentioned herein are indicative of the levels of skill of those skilled in the art to which the inventions pertain. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention, including the use of connexin hemichannel modulators, connexin 43 hemichannel modulators, inflammasome modulators, NLRP3 inflammasome modulators, pannexin channel modulators and/or pannexin 1 channel modulators other than those described or referenced herein. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, “containing”, etc. are to be read expansively and without limitation. It is understood that the methods and processes illustratively described herein suitably may be practiced in differing orders of steps, and that they are not necessarily restricted to the orders of steps indicated herein or in the claims. It is also understood that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of any patent or intellectual property office, including the United States Patent and Trademark Office, unless such statement is specifically and without qualification or reservation expressly adopted in a responsive writing by the inventors or their representative. Furthermore, titles, headings, or the like are provided to enhance the reader's comprehension of this document and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or embodiments of the invention are described in terms of Markush groups, those skilled in the art will recognize that the invention is also thereby described in terms of any individual member or subgroup of members of the Markush group.

We claim:

1. A method for treating one or more signs or symptoms of a post-acute infection disorder in a subject comprising administering to the subject an effective amount of a connexin 43 hemichannel modulator.

2. A method according to claim 1, wherein the connexin hemichannel modulator inhibits release of ATP from connexin 43 hemichannels.

3. A method according to claim 1, wherein that connexin hemichannel modulator inhibits NLRP3 inflammasome activity.

4. The method of claim 1, wherein the subject has or has been diagnosed with long COVID.

5. The method of claim 1, wherein one or more signs or symptoms of a post-acute infection disorder are relieved or improved.

6. The method of claim 5, wherein the post-acute infection disorder is long COVID and one or more signs or symptoms of long-COVID are relieved or improved.

7. The method of claim 6, wherein the signs or symptoms of long-COVID that are relieved or improved are one or more of fatigue, brain fog, trouble concentrating, peripheral neuropathy, muscle pain and/or joint pain.

8. The method of claim 5 or 6, wherein the one or more one or more signs or symptoms of a post-acute infection disorder comprises a brain or neurological sign or symptom.

9. The method of claim 8, wherein the brain or neurological sign or symptom is selected from the group consisting of brain fog, peripheral neuropathies (pins & needles/numbness any body part), dizziness, confusion, hallucinations, loss of smell and/or taste, phantom smells and/or taste, tinnitus (ringing in ears), internal vibrations/tremors, speech issues/stutter, stroke, seizures, and headaches.

10. The method of claim 6, wherein the one or more one or more signs or symptoms of long-COVID comprises an ocular sign or symptom.

11. The method of claim 10, wherein the ocular sign or symptom is selected from the group consisting of eye pain, burning eye pain, feeling of sand/glass in eyes, eye redness, vision changes, visual disturbances/floaters.

12. The method of claim 6, wherein the one or more one or more signs or symptoms of long-COVID comprises a breathing or respiratory sign or symptom.

13. The method of claim 12, wherein the breathing or respiratory sign or symptom is selected from the group consisting of shortness of breath, cough, post exertional malaise (tired after minor exertion), and fatigue or extreme fatigue/exhaustion without exertion.

14. The method of claim 6, wherein the one or more one or more signs or symptoms of long-COVID is a digestive or gastrointestinal sign or symptom.

15. The method of claim 14, wherein the digestive or gastrointestinal sign or symptom is selected from the group consisting of nausea, vomiting, diarrhea, bloating, and early satiety (feeling full after eating small amounts of food).

16. The method of claim 6, wherein the one or more one or more signs or symptoms of long-COVID comprises a cardiovascular sign or symptom.

17. The method of claim 16, wherein the cardiovascular sign or symptom is selected from the group consisting of heart arrythmias, tachycardia, bradycardia and chest pain.

18. The method of claim 6, wherein the one or more one or more signs or symptoms of long-COVID comprises a musculoskeletal sign or symptom.

19. The method of claim 18, wherein the musculoskeletal sign or symptom is selected from the group consisting of joint pain, muscle pain and bone pain.

20. The method of claim 6, wherein the one or more one or more signs or symptoms of long-COVID comprises a dermatological sign or symptom.

21. The method of claim 20, wherein the dermatological sign or symptom is selected from the group consisting of skin rashes and hair loss.

22. The method of claim 6, wherein the one or more one or more signs or symptoms of long-COVID comprises a mental health sign or symptom.

23. The method of claim 22, wherein the mental health sign or symptom is selected from the group consisting of anxiety and depression.

24. The method of claim 6, wherein the one or more one or more signs or symptoms of long-COVID comprises a sign or symptom selected from the group consisting of flushing of the face, facial paralysis, ptosis, bruising, cold hands and/or feet, heat and/or cold intolerance, feeling of fever without having one, and mottling of hands and/or feet.

25. The method of claim 1, wherein the connexin hemichannel modulator is a compound according to Formula I.

26. The method of claim 25, wherein the connexin hemichannel modulator is a compound according to Formula I is tonabersat.

27. The method of claim 26, wherein tonabersat is administered orally and the subject is a human.

28. The method of claim 1, wherein the connexin hemichannel modulator is a compound is a tonabersat prodrug.

29. The method of claim 27, wherein the tonabersat prodrug is a compound according to Formula II.

30. The method of claim 1, wherein the connexin hemichannel modulator is administered at least once per day.

31. The method of claim 1, wherein the connexin hemichannel modulator is administered orally, topically, or parenterally.

32. The method of claim 1, wherein the connexin hemichannel modulator is tonabersat and it is administered in an amount ranging from about 40 mg to 80 mg per dose.

33. The method of claim 32, wherein the connexin hemichannel modulator is administered once per day, twice per day, every other day, twice a week, or once a week.

34. The method of claim 1, where the subject is a human.

35. A method for treating one or more signs or symptoms of a post-acute infection disorder in a subject comprising administering to the subject a loading dose of a connexin 43 hemichannel modulator followed by the administration of a maintenance dose.

36. The method of claim 35 wherein, the post-acute infection disorder is long COVID.

37. The method of claim 36 wherein, the post-acute infection disorder is long COVID and one or more signs or symptoms of long-COVID are relieved or improved.

38. The method of claim 37, wherein the signs or symptoms of long-COVID that are relieved or improved are one or more of fatigue, brain fog, trouble concentrating, peripheral neuropathy, muscle pain and/or joint pain.

39. A method for preventing or ameliorating one or more signs or symptoms of a post-acute infection disorder in a subject comprising administering to the subject an effective amount of a connexin 43 hemichannel modulator.

40. A method according to claim 39, wherein the connexin hemichannel modulator inhibits release of ATP from connexin 43 hemichannels.

41. A method according to claim 39, wherein that connexin hemichannel modulator inhibits NLRP3 inflammasome activity.

42. The method of claim 39, wherein the subject has or has been diagnosed with long COVID, long flu, or post-Lyme disease treatment syndrome.


SEQUENCE LISTING (250 sequences)

SEQ ID NO: 1 - DNA, 30 bp, synthetic construct
  misc_feature 1..30: note = Description of Artificial Sequence: Syntheticoligonucleotide
  source 1..30: mol_type = other DNA
gtaattgcggcaagaagaattgtttctgtc

SEQ ID NO: 2 - DNA, 30 bp, synthetic construct
  misc_feature 1..30: note = Description of Artificial Sequence: Syntheticoligonucleotide
  source 1..30: mol_type = other DNA
gtaattgcggcaggaggaattgtttctgtc

SEQ ID NO: 3 - DNA, 30 bp, synthetic construct
  misc_feature 1..30: note = Description of Artificial Sequence: Syntheticoligonucleotide
  source 1..30: mol_type = other DNA
ggcaagagacaccaaagacactaccagcat

SEQ ID NO: 4 - DNA, 20 bp, synthetic construct
  misc_feature 1..20: note = Description of Artificial Sequence: Syntheticoligonucleotide
  source 1..20: mol_type = other DNA
ccaggctgactcaaccgctg

SEQ ID NO: 5 - DNA, 20 bp, synthetic construct
  misc_feature 1..20: note = Description of Artificial Sequence: Syntheticoligonucleotide
  source 1..20: mol_type = other DNA
acccatgttgcctgggcacc

SEQ ID NO: 6 - DNA, 20 bp, synthetic construct
  misc_feature 1..20: note = Description of Artificial Sequence: Syntheticoligonucleotide
  source 1..20: mol_type = other DNA
gtaggcttgaaccttgtcaa

SEQ ID NO: 7 - DNA, 20 bp, synthetic construct
  misc_feature 1..20: note = Description of Artificial Sequence: Syntheticoligonucleotide
  source 1..20: mol_type = other DNA
tctccccaggctgactcaac

SEQ ID NO: 8 - DNA, 20 bp, synthetic construct
  misc_feature 1..20: note = Description of Artificial Sequence: Syntheticoligonucleotide
  source 1..20: mol_type = other DNA
cagaagcgcacatgagagat

SEQ ID NO: 9 - DNA, 20 bp, synthetic construct
  misc_feature 1..20: note = Description of Artificial Sequence: Syntheticoligonucleotide
  source 1..20: mol_type = other DNA
gaagcgcacatgagagattg

SEQ ID NO: 10 - DNA, 20 bp, synthetic construct
  misc_feature 1..20: note = Description of Artificial Sequence: Syntheticoligonucleotide
  source 1..20: mol_type = other DNA
agtgtgggtacagacacaaa

SEQ ID NO: 11 - DNA, 20 bp, synthetic construct
  misc_feature 1..20: note = Description of Artificial Sequence: Syntheticoligonucleotide
  source 1..20: mol_type = other DNA
cagacacaaatatgatctgc

SEQ ID NO: 12 - DNA, 20 bp, synthetic construct
  misc_feature 1..20: note = Description of Artificial Sequence: Syntheticoligonucleotide
  source 1..20: mol_type = other DNA
atatgatctgcaggacccag

SEQ ID NO: 13 - DNA, 20 bp, synthetic construct
  misc_feature 1..20: note = Description of Artificial Sequence: Syntheticoligonucleotide
  source 1..20: mol_type = other DNA
gtaattgcggcaagaagaat

SEQ ID NO: 14 - DNA, 20 bp, synthetic construct
  misc_feature 1..20: note = Description of Artificial Sequence: Syntheticoligonucleotide
  source 1..20: mol_type = other DNA
aggctgtgcatgggagttag

SEQ ID NO: 15 - DNA, 20 bp, synthetic construct
  misc_feature 1..20: note = Description of Artificial Sequence: Syntheticoligonucleotide
  source 1..20: mol_type = other DNA
cgctggtccacaatggctag

SEQ ID NO: 16 - DNA, 20 bp, synthetic construct
  misc_feature 1..20: note = Description of Artificial Sequence: Syntheticoligonucleotide
  source 1..20: mol_type = other DNA
gctggctctgcttgaaggtc

SEQ ID NO: 17 - DNA, 3088 bp, Homo sapiens
  source 1..3088: mol_type = other DNA
acaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacat

SEQ ID NO: 18 - protein, 4 aa, synthetic construct
  REGION 1..4: note = Description of Artificial Sequence: Syntheticpeptide
SHVR

SEQ ID NO: 19 - protein, 382 aa, synthetic construct
  REGION 1..382: note = Description of Artificial Sequence: Syntheticpolypeptide
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 20 - protein, 6 aa, synthetic construct
  REGION 1..6: note = Description of Artificial Sequence: Syntheticpeptide
SRPTEK

SEQ ID NO: 21 - protein, 10 aa, synthetic construct
  REGION 1..10: note = Description of Artificial Sequence: Syntheticpeptide
WRQAAFVDSY

SEQ ID NO: 22 - protein, 13 aa, synthetic construct
  REGION 1..13: note = Description of Artificial Sequence: Syntheticpeptide
ICVVQDWGHHRCT

SEQ ID NO: 23 - DNA, 29 bp, synthetic construct
  misc_feature 1..29: note = Description of Artificial Sequence: Syntheticoligonucleotide
  source 1..29: mol_type = other DNA
gtaattgcggcaagaagaattgtttctgt

SEQ ID NO: 24 - DNA, 20 bp, synthetic construct
  misc_feature 1..20: note = Description of Artificial Sequence: Syntheticoligonucleotide
  source 1..20: mol_type = other DNA
acccatgttgcctgggcacc

SEQ ID NO: 25 - DNA, 20 bp, synthetic construct
  misc_feature 1..20: note = Description of Artificial Sequence: Syntheticoligonucleotide
  source 1..20: mol_type = other DNA
gttgcctgggcaccactctt

SEQ ID NO: 26 - DNA, 20 bp, synthetic construct
  misc_feature 1..20: note = Description of Artificial Sequence: Syntheticoligonucleotide
  source 1..20: mol_type = other DNA
gcctgggcaccactcttttg

SEQ ID NO: 27 - DNA, 20 bp, synthetic construct
  misc_feature 1..20: note = Description of Artificial Sequence: Syntheticoligonucleotide
  source 1..20: mol_type = other DNA
tgggcaccactcttttgctt

SEQ ID NO: 28 - DNA, 20 bp, synthetic construct
  misc_feature 1..20: note = Description of Artificial Sequence: Syntheticoligonucleotide
  source 1..20: mol_type = other DNA
gtaggcttgaaccttgtcaa

SEQ ID NO: 29 - DNA, 20 bp, synthetic construct
  misc_feature 1..20: note = Description of Artificial Sequence: Syntheticoligonucleotide
  source 1..20: mol_type = other DNA
tctccccaggctgactcaac

SEQ ID NO: 30 - DNA, 20 bp, synthetic construct
  misc_feature 1..20: note = Description of Artificial Sequence: Syntheticoligonucleotide
  source 1..20: mol_type = other DNA
ccaggctgactcaaccgctg

SEQ ID NO: 31 - DNA, 20 bp, synthetic construct
  misc_feature 1..20: note = Description of Artificial Sequence: Syntheticoligonucleotide
  source 1..20: mol_type = other DNA
ctcaaccgctgtccccagca

SEQ ID NO: 32 - DNA, 20 bp, synthetic construct
  misc_feature 1..20: note = Description of Artificial Sequence: Syntheticoligonucleotide
  source 1..20: mol_type = other DNA
cagaagcgcacatgagagat

SEQ ID NO: 33 - DNA, 20 bp, synthetic construct
  misc_feature 1..20: note = Description of Artificial Sequence: Syntheticoligonucleotide
  source 1..20: mol_type = other DNA
gaagcgcacatgagagattg

SEQ ID NO: 34 - DNA, 20 bp, synthetic construct
  misc_feature 1..20: note = Description of Artificial Sequence: Syntheticoligonucleotide
  source 1..20: mol_type = other DNA
agtgtgggtacagacacaaa

SEQ ID NO: 35 - DNA, 20 bp, synthetic construct
  misc_feature 1..20: note = Description of Artificial Sequence: Syntheticoligonucleotide
  source 1..20: mol_type = other DNA
tgggtacagacacaaatatg

SEQ ID NO: 36 - DNA, 20 bp, synthetic construct
  misc_feature 1..20: note = Description of Artificial Sequence: Syntheticoligonucleotide
  source 1..20: mol_type = other DNA
ggtacagacacaaatatgat

SEQ ID NO: 37 - DNA, 20 bp, synthetic construct
  misc_feature 1..20: note = Description of Artificial Sequence: Syntheticoligonucleotide
  source 1..20: mol_type = other DNA
cagacacaaatatgatctgc

SEQ ID NO: 38 - DNA, 20 bp, synthetic construct
  misc_feature 1..20: note = Description of Artificial Sequence: Syntheticoligonucleotide
  source 1..20: mol_type = other DNA
gacacaaatatgatctgcag

SEQ ID NO: 39 - DNA, 20 bp, synthetic construct
  misc_feature 1..20: note = Description of Artificial Sequence: Syntheticoligonucleotide
  source 1..20: mol_type = other DNA
acaaatatgatctgcaggac

SEQ ID NO: 40 - DNA, 20 bp, synthetic construct
  misc_feature 1..20: note = Description of Artificial Sequence: Syntheticoligonucleotide
  source 1..20: mol_type = other DNA
atatgatctgcaggacccag

SEQ ID NO: 41 - DNA, 20 bp, synthetic construct
  misc_feature 1..20: note = Description of Artificial Sequence: Syntheticoligonucleotide
  source 1..20: mol_type = other DNA
atgatgaagatggttttctc

SEQ ID NO: 42 - DNA, 20 bp, synthetic construct
  misc_feature 1..20: note = Description of Artificial Sequence: Syntheticoligonucleotide
  source 1..20: mol_type = other DNA
atgaagatgatgaagatggt

SEQ ID NO: 43 - DNA, 20 bp, synthetic construct
  misc_feature 1..20: note = Description of Artificial Sequence: Syntheticoligonucleotide
  source 1..20: mol_type = other DNA
agcatgaagatgatgaagat

SEQ ID NO: 44 - DNA, 20 bp, synthetic construct
  misc_feature 1..20: note = Description of Artificial Sequence: Syntheticoligonucleotide
  source 1..20: mol_type = other DNA
atgaagatggttttctccgt

SEQ ID NO: 45 - DNA, 20 bp, synthetic construct
  misc_feature 1..20: note = Description of Artificial Sequence: Syntheticoligonucleotide
  source 1..20: mol_type = other DNA
aggctgtgcatgggagttag

SEQ ID NO: 46 - DNA, 20 bp, synthetic construct
  misc_feature 1..20: note = Description of Artificial Sequence: Syntheticoligonucleotide
  source 1..20: mol_type = other DNA
cgctggtccacaatggctag

SEQ ID NO: 47 - DNA, 20 bp, synthetic construct
  misc_feature 1..20: note = Description of Artificial Sequence: Syntheticoligonucleotide
  source 1..20: mol_type = other DNA
gctggctctgcttgaaggtc

SEQ ID NO: 48 - DNA, 20 bp, synthetic construct
  misc_feature 1..20: note = Description of Artificial Sequence: Syntheticoligonucleotide
  source 1..20: mol_type = other DNA
ggtgcccaggcaacatgggt

SEQ ID NO: 49 - DNA, 20 bp, synthetic construct
  misc_feature 1..20: note = Description of Artificial Sequence: Syntheticoligonucleotide
  source 1..20: mol_type = other DNA
aagagtggtgcccaggcaac

SEQ ID NO: 50 - DNA, 20 bp, synthetic construct
  misc_feature 1..20: note = Description of Artificial Sequence: Syntheticoligonucleotide
  source 1..20: mol_type = other DNA
caaaagagtggtgcccaggc

SEQ ID NO: 51 - DNA, 20 bp, synthetic construct
  misc_feature 1..20: note = Description of Artificial Sequence: Syntheticoligonucleotide
  source 1..20: mol_type = other DNA
agcaaaagagtggtgcccag

SEQ ID NO: 52 - DNA, 20 bp, synthetic construct
  misc_feature 1..20: note = Description of Artificial Sequence: Syntheticoligonucleotide
  source 1..20: mol_type = other DNA
cttgacaaggttcaagccta

SEQ ID NO: 53 - DNA, 20 bp, synthetic construct
  misc_feature 1..20: note = Description of Artificial Sequence: Syntheticoligonucleotide
  source 1..20: mol_type = other DNA
gttgagtcagcctggggaga

SEQ ID NO: 54 - DNA, 20 bp, synthetic construct
  misc_feature 1..20: note = Description of Artificial Sequence: Syntheticoligonucleotide
  source 1..20: mol_type = other DNA
cagcggttgagtcagcctgg

SEQ ID NO: 55 - DNA, 20 bp, synthetic construct
  misc_feature 1..20: note = Description of Artificial Sequence: Syntheticoligonucleotide
  source 1..20: mol_type = other DNA
tgctggggacagcggttgag

SEQ ID NO: 56 - DNA, 20 bp, synthetic construct
  misc_feature 1..20: note = Description of Artificial Sequence: Syntheticoligonucleotide
  source 1..20: mol_type = other DNA
atctctcatgtgcgcttctg

SEQ ID NO: 57 - DNA, 20 bp, synthetic construct
  misc_feature 1..20: note = Description of Artificial Sequence: Syntheticoligonucleotide
  source 1..20: mol_type = other DNA
caatctctcatgtgcgcttc

SEQ ID NO: 58 - DNA, 20 bp, synthetic construct
  misc_feature 1..20: note = Description of Artificial Sequence: Syntheticoligonucleotide
  source 1..20: mol_type = other DNA
tttgtgtctgtacccacact

SEQ ID NO: 59 - DNA, 20 bp, synthetic construct
  misc_feature 1..20: note = Description of Artificial Sequence: Syntheticoligonucleotide
  source 1..20: mol_type = other DNA
catatttgtgtctgtaccca

SEQ ID NO: 60 - DNA, 20 bp, synthetic construct
  misc_feature 1..20: note = Description of Artificial Sequence: Syntheticoligonucleotide
  source 1..20: mol_type = other DNA
atcatatttgtgtctgtacc

SEQ ID NO: 61 - DNA, 20 bp, synthetic construct
  misc_feature 1..20: note = Description of Artificial Sequence: Syntheticoligonucleotide
  source 1..20: mol_type = other DNA
gcagatcatatttgtgtctg

SEQ ID NO: 62 - DNA, 20 bp, synthetic construct
  misc_feature 1..20: note = Description of Artificial Sequence: Syntheticoligonucleotide
  source 1..20: mol_type = other DNA
ctgcagatcatatttgtgtc

SEQ ID NO: 63 - DNA, 20 bp, synthetic construct
  misc_feature 1..20: note = Description of Artificial Sequence: Syntheticoligonucleotide
  source 1..20: mol_type = other DNA
gtcctgcagatcatatttgt

SEQ ID NO: 64 - DNA, 20 bp, synthetic construct
  misc_feature 1..20: note = Description of Artificial Sequence: Syntheticoligonucleotide
  source 1..20: mol_type = other DNA
ctgggtcctgcagatcatat

SEQ ID NO: 65 - DNA, 20 bp, synthetic construct
  misc_feature 1..20: note = Description of Artificial Sequence: Syntheticoligonucleotide
  source 1..20: mol_type = other DNA
gagaaaaccatcttcatcat

SEQ ID NO: 66 - DNA, 20 bp, synthetic construct
  misc_feature 1..20: note = Description of Artificial Sequence: Syntheticoligonucleotide
  source 1..20: mol_type = other DNA
accatcttcatcatcttcat

SEQ ID NO: 67 - DNA, 20 bp, synthetic construct
  misc_feature 1..20: note = Description of Artificial Sequence: Syntheticoligonucleotide
  source 1..20: mol_type = other DNA
atcttcatcatcttcatgct

SEQ ID NO: 68 - DNA, 20 bp, synthetic construct
  misc_feature 1..20: note = Description of Artificial Sequence: Syntheticoligonucleotide
  source 1..20: mol_type = other DNA
acggagaaaaccatcttcat

SEQ ID NO: 69 - DNA, 20 bp, synthetic construct
  misc_feature 1..20: note = Description of Artificial Sequence: Syntheticoligonucleotide
  source 1..20: mol_type = other DNA
ctaactcccatgcacagcct

SEQ ID NO: 70 - DNA, 20 bp, synthetic construct
  misc_feature 1..20: note = Description of Artificial Sequence: Syntheticoligonucleotide
  source 1..20: mol_type = other DNA
ctagccattgtggaccagcg

SEQ ID NO: 71 - DNA, 20 bp, synthetic construct
  misc_feature 1..20: note = Description of Artificial Sequence: Syntheticoligonucleotide
  source 1..20: mol_type = other DNA
gaccttcaagcagagccagc

SEQ ID NO: 72 - protein, 46 aa, synthetic construct
  REGION 1..46: note = Description of Artificial Sequence: Syntheticpolypeptide
LTAVGGESIYYDEQSKFVCNTEQPGCENVCYDAFAPLSHVRFWVFQ

SEQ ID NO: 73 - protein, 15 aa, synthetic construct
  REGION 1..15: note = Description of Artificial Sequence: Syntheticpeptide
LTAVGGESIYYDEQS

SEQ ID NO: 74 - protein, 13 aa, synthetic construct
  REGION 1..13: note = Description of Artificial Sequence: Syntheticpeptide
DEQSKFVCNTEQP

SEQ ID NO: 75 - protein, 13 aa, synthetic construct
  REGION 1..13: note = Description of Artificial Sequence: Syntheticpeptide
TEQPGCENVCYDA

SEQ ID NO: 76 - protein, 13 aa, synthetic construct
  REGION 1..13: note = Description of Artificial Sequence: Syntheticpeptide
VCYDAFAPLSHVR

SEQ ID NO: 77 - protein, 12 aa, synthetic construct
  REGION 1..12: note = Description of Artificial Sequence: Syntheticpeptide
APLSHVRFWVFQ

SEQ ID NO: 78 - protein, 47 aa, synthetic construct
  REGION 1..47: note = Description of Artificial Sequence: Syntheticpolypeptide
FEVGFLIGQYFLYGFQVHPFYVCSRLPCHPKIDCFISRPTEKTIFLL

SEQ ID NO: 79 - protein, 11 aa, synthetic construct
  REGION 1..11: note = Description of Artificial Sequence: Syntheticpeptide
FEVGFLIGQYF

SEQ ID NO: 80 - protein, 12 aa, synthetic construct
  REGION 1..12: note = Description of Artificial Sequence: Syntheticpeptide
LIGQYFLYGFQV

SEQ ID NO: 81 - protein, 14 aa, synthetic construct
  REGION 1..14: note = Description of Artificial Sequence: Syntheticpeptide
GFQVHPFYVCSRLP

SEQ ID NO: 82 - protein, 12 aa, synthetic construct
  REGION 1..12: note = Description of Artificial Sequence: Syntheticpeptide
SRLPCHPKIDCF

SEQ ID NO: 83 - protein, 12 aa, synthetic construct
  REGION 1..12: note = Description of Artificial Sequence: Syntheticpeptide
IDCFISRPTEKT

SEQ ID NO: 84 - protein, 11 aa, synthetic construct
  REGION 1..11: note = Description of Artificial Sequence: Syntheticpeptide
SRPTEKTIFLL

SEQ ID NO: 85 - protein, 11 aa, synthetic construct
  REGION 1..11: note = Description of Artificial Sequence: Syntheticpeptide
SRPTEKTIFII

SEQ ID NO: 86 - protein, 10 aa, synthetic construct
  REGION 1..10: note = Description of Artificial Sequence: Syntheticpeptide
YVCSRLPCHP

SEQ ID NO: 87 - protein, 11 aa, synthetic construct
  REGION 1..11: note = Description of Artificial Sequence: Syntheticpeptide
QVHPFYVCSRL

SEQ ID NO: 88 - protein, 13 aa, synthetic construct
  REGION 1..13: note = Description of Artificial Sequence: Syntheticpeptide
FEVGFLIGQYFLY

SEQ ID NO: 89 - protein, 12 aa, synthetic construct
  REGION 1..12: note = Description of Artificial Sequence: Syntheticpeptide
GQYFLYGFQVHP

SEQ ID NO: 90 - protein, 12 aa, synthetic construct
  REGION 1..12: note = Description of Artificial Sequence: Syntheticpeptide
GFQVHPFYVCSR

SEQ ID NO: 91 - protein, 12 aa, synthetic construct
  REGION 1..12: note = Description of Artificial Sequence: Syntheticpeptide
AVGGESIYYDEQ

SEQ ID NO: 92 - protein, 12 aa, synthetic construct
  REGION 1..12: note = Description of Artificial Sequence: Syntheticpeptide
YDEQSKFVCNTE

SEQ ID NO: 93 - protein, 12 aa, synthetic construct
  REGION 1..12: note = Description of Artificial Sequence: Syntheticpeptide
NTEQPGCENVCY

SEQ ID NO: 94 - protein, 12 aa, synthetic construct
  REGION 1..12: note = Description of Artificial Sequence: Syntheticpeptide
CYDAFAPLSHVR

SEQ ID NO: 95 - protein, 12 aa, synthetic construct
  REGION 1..12: note = Description of Artificial Sequence: Syntheticpeptide
FAPLSHVRFWVF

SEQ ID NO: 96 - protein, 5 aa, synthetic construct
  REGION 1..5: note = Description of Artificial Sequence: Syntheticpeptide
LIGQY

SEQ ID NO: 97 - protein, 5 aa, synthetic construct
  REGION 1..5: note = Description of Artificial Sequence: Syntheticpeptide
QVHPF

SEQ ID NO: 98 - protein, 5 aa, synthetic construct
  REGION 1..5: note = Description of Artificial Sequence: Syntheticpeptide
YVCSR

SEQ ID NO: 99 - protein, 5 aa, synthetic construct
  REGION 1..5: note = Description of Artificial Sequence: Syntheticpeptide
SRLPC

SEQ ID NO: 100 - protein, 382 aa, Homo sapiens; identical to SEQ ID NO: 19

SEQ ID NO: 101 - protein, 7 aa, synthetic construct
SRPTEKT

SEQ ID NO: 102 - protein, 13 aa, synthetic construct
VCYDKSFPISHVR

SEQ ID NO: 103 - protein, 12 aa, synthetic construct
INCTLQPGCNSV

SEQ ID NO: 104 - protein, 11 aa, synthetic construct
SRPTEKTIFII

SEQ ID NO: 105 - protein, 26 aa, synthetic construct
GGERAPLAADQGSVKKSSSSSKGTKK

SEQ ID NO: 106 - protein, 10 aa, synthetic construct
SRARSDDLTV

SEQ ID NO: 107 - protein, 12 aa, synthetic construct
VDCFLSRPTEKT

SEQ ID NO: 108 - protein, 9 aa, synthetic construct
KQIEIKKFK

SEQ ID NO: 109 - protein, 11 aa, synthetic construct
SRPTEKTIFFI

SEQ ID NO: 110 - protein, 9 aa, synthetic construct
SRPTEKTIF

SEQ ID NO: 111 - protein, 17 aa, synthetic construct
  MOD_RES 1..6: note = D-isomer
LCLRPVGGKQIEIKKFK

SEQ ID NO: 112 - protein, 12 aa, synthetic construct
ADCFLSRPTEKT

SEQ ID NO: 113 - protein, 12 aa, synthetic construct
VACFLSRPTEKT

SEQ ID NO: 114 - protein, 12 aa, synthetic construct
VDCFLSRPTAKT

SEQ ID NO: 115 - protein, 12 aa, synthetic construct
VDCFLSRPTEAT

SEQ ID NO: 116 - protein, 10 aa, synthetic construct
CFLSRPTEKT

SEQ ID NO: 117 - protein, 8 aa, synthetic construct
LSRPTEKT

SEQ ID NO: 118 - protein, 11 aa, synthetic construct
DCFISRPTEKT

SEQ ID NO: 119 - protein, 26 aa, synthetic construct
DGVNVEMHLKQIEIKKFKYGIEEHGK

SEQ ID NO: 120 - protein, 26 aa, synthetic construct
DGVNVEMHLKQIEIKKFKYGIEEQGK

SEQ ID NO: 121 - protein, 20 aa, synthetic construct
YGRKKRRQRRRKQIEIKKFK

SEQ ID NO: 122 - protein, 17 aa, synthetic construct
CSSPTAPLSPMSPPGYK

SEQ ID NO: 123 - protein, 11 aa, synthetic construct
PTAPLSPMSPP

SEQ ID NO: 124 - protein, 7 aa, synthetic construct
RPRDDEI

SEQ ID NO: 125 - protein, 9 aa, synthetic construct
SRPRDDLEI

SEQ ID NO: 126 - protein, 19 aa, synthetic construct
YGRKKRRQRRRSRPRDDEI

SEQ ID NO: 127 - protein, 18 aa, synthetic construct
YGRKKRRQRRRRPRDDEI

SEQ ID NO: 128 - protein, 6 aa, synthetic construct
  MOD_RES 4: note = 4Hyp
GAGXPY

SEQ ID NO: 129 - protein, 6 aa, synthetic construct
  MOD_RES 3: note = D-4Hyp
  MOD_RES 1: note = D-Tyr
  MOD_RES 5: note = D-Ala
YPXGAG

SEQ ID NO: 130 - protein, 35 aa, synthetic construct
ESAWGDEQSAFRCNTQQPGCENVCYDKSFPISHVR

SEQ ID NO: 131 - protein, 36 aa, synthetic construct
GESIYYDEQSKFVCNTEQPGCENVCYDAFAPLSHVR

SEQ ID NO: 132 - protein, 38 aa, synthetic construct
LLIQWYIYGFSLSAVYTCKRDPCPHQVDCFLSRPTEKT

SEQ ID NO: 133 - protein, 38 aa, synthetic construct
LIGQYFLYGFQVHPFYVCSRLPCHPKIDCFISRPTEKT

SEQ ID NO: 134 - protein, 15 aa, synthetic construct
VFFKGVKDRVKGRSD

SEQ ID NO: 135 - protein, 8 aa, synthetic construct
RPRDDLEI

SEQ ID NO: 136 - protein, 9 aa, synthetic construct
SRPRDDLEI

SEQ ID NO: 137 - protein, 25 aa, synthetic construct
RQPKIWFPNRRKPWKKRPRPDDLEI

SEQ ID NO: 138 - protein, 13 aa, synthetic construct
GHGDPLHLEEVKC

SEQ ID NO: 139 - protein, 24 aa, synthetic construct
YGRKKRRQRRRGHGDPLHLEEVKC

SEQ ID NO: 140 - protein, 20 aa, synthetic construct
PSSRASSRASSRPRPDDLEI

SEQ ID NO: 141 - protein, 9 aa, synthetic construct
RPRPDDLEI

SEQ ID NO: 142 - protein, 9 aa, synthetic construct
RPRPDDLEV

SEQ ID NO: 143 - protein, 9 aa, synthetic construct
RPRPDDVPV

SEQ ID NO: 144 - protein, 9 aa, synthetic construct
KARSDDLSV

SEQ ID NO: 145 - protein, 40 aa, synthetic construct
QKPEVPNGVSPGHRLPHGYHSDKRRLSKASSKARSDDLSV

SEQ ID NO: 146 - protein, 36 aa, synthetic construct
RQPKIWFPNRRKPWKKPSSRASSRASSRPRPDDLEI

SEQ ID NO: 147 - protein, 36 aa, synthetic construct; identical to SEQ ID NO: 146

SEQ ID NO: 148 - protein, 25 aa, synthetic construct
RQPKIWFPNRRKPWKKRPRPDDLEI

SEQ ID NO: 149 - protein, 25 aa, synthetic construct
RQPKIWFPNRRKPWKKRPRPDDLEV

SEQ ID NO: 150 - protein, 25 aa, synthetic construct
RQPKIWFPNRRKPWKKRPRPDDVPV

SEQ ID NO: 151 - protein, 25 aa, synthetic construct
RQPKIWFPNRRKPWKKKARSDDLSV

SEQ ID NO: 152 - protein, 9 aa, synthetic construct
RPKPDDLDI

SEQ ID NO: 153 - protein, 20 aa, synthetic construct
GRKKRRQRPPQRPRPDDLEI

SEQ ID NO: 154 - protein, 25 aa, synthetic construct
RQIKIWFQNRRMKWKKRPRPDDLEI

SEQ ID NO: 155 - protein, 25 aa, synthetic construct
RQIAIWFQNRRMKWAARPRPDDLEI

SEQ ID NO: 156 - protein, 18 aa, synthetic construct
RKKRRQRRRRPRPDDLEI

SEQ ID NO: 157 - protein, 30 aa, synthetic construct
TRSSRAGLQFPVGRVHRLLRKRPRPDDLEI

SEQ ID NO: 158 - protein, 35 aa, synthetic construct
GWTLNSAGYLLGKINKALAALAKKILRPRPDDLEI

SEQ ID NO: 159 - protein, 27 aa, synthetic construct
KLALKLALKALKAALKLARPRPDDLEI

SEQ ID NO: 160 - protein, 25 aa, synthetic construct
AAVALLPAVLLALLAPRPRPDDLEI

SEQ ID NO: 161 - protein, 19 aa, synthetic construct
VPMLKPMLKERPRPDDLEI

SEQ ID NO: 162 - protein, 37 aa, synthetic construct
MANLGYWLLALFVTMWTDVGLCKKRPKPRPRPDDLEI

SEQ ID NO: 163 - protein, 27 aa, synthetic construct
LLIILRRRIRKQAHAHSKRPRPDDLEI

SEQ ID NO: 164 - protein, 30 aa, synthetic construct
KETWWETWWTEWSQPKKKRKVRPRPDDLEI

SEQ ID NO: 165 - protein, 27 aa, synthetic construct
RGGRLSYSRRRFSTSTGRRPRPDDLEI

SEQ ID NO: 166 - protein, 24 aa, synthetic construct
SDLWEMMMVSLACQYRPRPDDLEI

SEQ ID NO: 167 - protein, 21 aa, synthetic construct
TSPLNIHNGQKLRPRPDDLEI

SEQ ID NO: 168 - protein, 28 aa, synthetic construct
RVIRVWFQNKRCKDKKVDCFLSRPTEKT

SEQ ID NO: 169 - protein, 39 aa, synthetic construct
GALFLGFLGAAGSTMGAWSQPKKKRKVVDCFLSRPTEKT

SEQ ID NO: 170 - protein, 39 aa, synthetic construct
GALFLAFLAAALSLMGLWSQPKKKRRVVDCFLSRPTEKT

SEQ ID NO: 171 - protein, 39 aa, synthetic construct
MYVFYVMYDGFSMQRLVKCNAWPCPNTVDCFVSRPTEKT

SEQ ID NO: 172 - protein, 39 aa, synthetic construct
MYVFYFLYNGYHLPWVLKCGIDPCPNLVDCFISRPTEKT

SEQ ID NO: 173 - protein, 39 aa, synthetic construct
LYIFHRLYKDYDMPRVVACSVEPCPHTVDCYISRPTEKK

SEQ ID NO: 174 - protein, 10 aa, synthetic construct
VDCFLSRPTE

SEQ ID NO: 175 - protein, 8 aa, synthetic construct
VDCFLSRP

SEQ ID NO: 176 - protein, 6 aa, synthetic construct
VDCFLS

SEQ ID NO: 177 - protein, 23 aa, synthetic construct
GRKKRRQRPPQVDCFLSRPTEKT

SEQ ID NO: 178 - protein, 28 aa, synthetic construct
RQIKIWFQNRRMKWKKVDCFLSRPTEKT

SEQ ID NO: 179 - protein, 28 aa, synthetic construct
RQIAIWFQNRRMKWAAVDCFLSRPTEKT

SEQ ID NO: 180 - protein, 21 aa, synthetic construct
RKKRRQRRRVDCFLSRPTEKT

SEQ ID NO: 181 - protein, 33 aa, synthetic construct
TRSSRAGLQFPVGRVHRLLRKVDCFLSRPTEKT

SEQ ID NO: 182 - protein, 38 aa, synthetic construct
GWTLNSAGYLLGKINKALAALAKKILVDCFLSRPTEKT

SEQ ID NO: 183 - protein, 30 aa, synthetic construct
KLALKLALKALKAALKLAVDCFLSRPTEKT

SEQ ID NO: 184 - protein, 28 aa, synthetic construct
AAVALLPAVLLALLAPVDCFLSRPTEKT

SEQ ID NO: 185 - protein, 22 aa, synthetic construct
VPMLKPMLKEVDCFLSRPTEKT

SEQ ID NO: 186 - protein, 40 aa, synthetic construct
MANLGYWLLALFVTMWTDVGLCKKRPKPVDCFLSRPTEKT

SEQ ID NO: 187 - protein, 30 aa, synthetic construct
LLIILRRRIRKQAHAHSKVDCFLSRPTEKT

SEQ ID NO: 188 - protein, 33 aa, synthetic construct
KETWWETWWTEWSQPKKKRKVVDCFLSRPTEKT

SEQ ID NO: 189 - protein, 30 aa, synthetic construct
RGGRLSYSRRRFSTSTGRVDCFLSRPTEKT

SEQ ID NO: 190 - protein, 27 aa, synthetic construct
SDLWEMMMVSLACQYVDCFLSRPTEKT

SEQ ID NO: 191 - protein, 24 aa, synthetic construct
TSPLNIHNGQKLVDCFLSRPTEKT

SEQ ID NO: 192 - protein, 11 aa, synthetic construct
FEVAFLLIQWI

SEQ ID NO: 193 - protein, 11 aa, synthetic construct
LLIQWYIGFSL

SEQ ID NO: 194 - protein, 16 aa, synthetic construct
SLSAVYTCKRDPCPHQ

SEQ ID NO: 195 - protein, 40 aa, synthetic construct
LYLLHTLWHGFNMPRLVQCANVAPCPNIVDCYIARPTEKK

SEQ ID NO: 196 - protein, 13 aa, synthetic construct
LGTAVESAWGDEQ

SEQ ID NO: 197 - protein, 12 aa, synthetic construct
QSAFRCNTQQPG

SEQ ID NO: 198 - protein, 12 aa, synthetic construct
QQPGCENVCYDK

SEQ ID NO: 199 - protein, 18 aa, synthetic construct
KRDPCHQVDCFLSRPTEK

SEQ ID NO: 200 - protein, 39 aa, synthetic construct
LYVFHSFYPKYILPPVVKCHADPCPNIVDCFISKPSEKN

SEQ ID NO: 201 - protein, 39 aa, synthetic construct
MYVFYLLYPGYAMVRLVKCDVYPCPNTVDCFVSRPTEKT

SEQ ID NO: 202 - protein, 32 aa, synthetic construct
LYGWTMEPVFVCQRAPCPYLVDCFVSRPTEKT

SEQ ID NO: 203 - protein, 32 aa, synthetic construct; identical to SEQ ID NO: 202

SEQ ID NO: 204 - protein, 38 aa, synthetic construct
GALHYFLFGFLAPKKFPCTRPPCTGVVDCYVSRPTEKS

SEQ ID NO: 205 - protein, 38 aa, synthetic construct
IAGQYFLYGFELKPLYRCDRWPCPNTVDCFISRPTEKT

SEQ ID NO: 206 - protein, 38 aa, synthetic construct
LVGQYLLYGFEVRPFFPCSRQPCPHVVDCFVSRPTEKT

SEQ ID NO: 207 - protein, 38 aa, synthetic construct
IVGQYFIYGIFLTTLHVCRRSPCPHPVNCYVSRPTEKN

SEQ ID NO: 208 - protein, 46 aa, synthetic construct; identical to SEQ ID NO: 72

SEQ ID NO: 209 - protein, 15 aa, synthetic construct
LTAVGGESIYYDEQS

SEQ ID NO: 210 - protein, 13 aa, synthetic construct
DEQSKFVCNTEQP

SEQ ID NO: 211 - protein, 13 aa, synthetic construct
TEQPGCENVCYDA

SEQ ID NO: 212 - protein, 13 aa, synthetic construct
VCYDAFAPLSHVR

SEQ ID NO: 213 - protein, 12 aa, synthetic construct
APLSHVRFWVFQ

SEQ ID NO: 214 - protein, 11 aa, synthetic construct
FEVGFLIGQYF

SEQ ID NO: 215 - protein, 12 aa, synthetic construct
LIGQYFLYGFQV

SEQ ID NO: 216 - protein, 14 aa, synthetic construct
GFQVHPFYVCSRLP

SEQ ID NO: 217 - protein, 12 aa, synthetic construct
SRLPCHPKIDCF

SEQ ID NO: 218 - protein, 12 aa, synthetic construct
IDCFISRPTEKT

SEQ ID NO: 219 - protein, 11 aa, synthetic construct
SRPTEKTIFLL

SEQ ID NO: 220 - protein, 10 aa, synthetic construct
YVCSRLPCHP

SEQ ID NO: 221 - protein, 11 aa, synthetic construct
QVHPFYVCSRL

SEQ ID NO: 222 - protein, 13 aa, synthetic construct
FEVGFLIGQYFLY

SEQ ID NO: 223 - protein, 12 aa, synthetic construct
GQYFLYGFQVHP

SEQ ID NO: 224 - protein, 12 aa, synthetic construct
GFQVHPFYVCSR

SEQ ID NO: 225 - protein, 12 aa, synthetic construct
AVGGESIYYDEQ

SEQ ID NO: 226 - protein, 12 aa, synthetic construct
YDEQSKFVCNTE

SEQ ID NO: 227 - protein, 12 aa, synthetic construct
NTEQPGCENVCY

SEQ ID NO: 228 - protein, 12 aa, synthetic construct
CYDAFAPLSHVR

SEQ ID NO: 229 - protein, 12 aa, synthetic construct
FAPLSHVRFWVF

SEQ ID NO: 230 - protein, 5 aa, synthetic construct
LIGQY

SEQ ID NO: 231 - protein, 5 aa, synthetic construct
QVHPF

SEQ ID NO: 232 - protein, 5 aa, synthetic construct
YVCSR

SEQ ID NO: 233 - protein, 5 aa, synthetic construct
SRLPC

SEQ ID NO: 234 - protein, 5 aa, synthetic construct
LPCHP

SEQ ID NO: 235 - protein, 5 aa, synthetic construct
GESIY

SEQ ID NO: 236 - protein, 6 aa, synthetic construct
YDEQSK

SEQ ID NO: 237 - protein, 6 aa, synthetic construct
SKFVCN

SEQ ID NO: 238 - protein, 8 aa, synthetic construct
TEQPGCEN

SEQ ID NO: 239 - protein, 8 aa, synthetic construct
VCYDAFAP

SEQ ID NO: 240 - protein, 10 aa, synthetic construct
LSHVRFWVFQ

SEQ ID NO: 241 - protein, 14 aa, synthetic construct
LIQYFLYGFQVHPF

SEQ ID NO: 242 - protein, 13 aa, synthetic construct
VHPFYCSRLPCHP

SEQ ID NO: 243 - protein, 22 aa, synthetic construct
VGGESIYYDEQSKFVCNTEQPG

SEQ ID NO: 244 - protein, 22 aa, synthetic construct
TEQPGCENVCYDAFAPLSHVRF

SEQ ID NO: 245 - protein, 14 aa, synthetic construct
AFAPLSHVRFWVFQ

SEQ ID NO: 246 - protein, 16 aa, synthetic construct
IDCFISRPTEKTIFLL

SEQ ID NO: 247 - RNA, 20 nt, synthetic construct
  source 1..20: mol_type = other RNA
  modified_base 1: mod_base = OTHER; note = thymine
  modified_base 3: mod_base = OTHER; note = thymine
  modified_base 14: mod_base = OTHER; note = thymine
tatgcagccacagtgggagg

SEQ ID NO: 248 - RNA, 20 nt, synthetic construct
  source 1..20: mol_type = other RNA
  modified_base 6: mod_base = OTHER; note = thymine
  modified_base 10: mod_base = OTHER; note = thymine
  modified_base 20: mod_base = OTHER; note = thymine
tcagatacctcccacaaact

SEQ ID NO: 249 - RNA, 21 nt, synthetic construct
  source 1..21: mol_type = other RNA
  modified_base 1: mod_base = OTHER; note = thymine
  modified_base 8: mod_base = OTHER; note = thymine
  modified_base 14: mod_base = OTHER; note = thymine
  modified_base 21: mod_base = OTHER; note = thymine
tagcacctgccagtccagaat

SEQ ID NO: 250 - RNA, 21 nt, synthetic construct
  source 1..21: mol_type = other RNA
  modified_base 3: mod_base = OTHER; note = thymine
  modified_base 9: mod_base = OTHER; note = thymine
  modified_base 13: mod_base = OTHER; note = thymine
  modified_base 15: mod_base = OTHER; note = thymine
  modified_base 17: mod_base = OTHER; note = thymine
ggtccaggtccatctctcagg